SEQUENCE LISTING (207 sequences)

SEQ ID NO: 1 - protein, 15 aa, synthetic construct
GGGGSGGGGSGGGGS

SEQ ID NO: 2 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 3 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 4 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 5 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 6 - protein, 118 aa, synthetic construct
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSS

SEQ ID NO: 7 - protein, 122 aa, synthetic construct
HVQLVESGGGLVQPGRSLRLSCAASGFTFSSYWMYWVRQTPGKGLEWVSTINRDGSATWYADSVKGRFTISRDNAKNTGYLQMNSLEPDDTAVYYCVSDPDNYSSDEMVPYWGQGTQVTVSS

SEQ ID NO: 8 - protein, 122 aa, synthetic construct
QVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 9 - protein, 122 aa, synthetic construct
  VARIANT 1: note = Any amino acid
  VARIANT 16: note = Any amino acid
  VARIANT 87: note = Any amino acid
XVQLVESGGGLVQPGXSLRLSCAASGFTFSSYWMYWVRQTPGKGLEWVSTINRDGSATWYADSVKGRFTISRDNAKNTGYLQMNSLXPDDTAVYYCVSDPDNYSSDEMVPYWGQGTQVTVSS

SEQ ID NO: 10 - protein, 130 aa, synthetic construct
QVQLVESGGGLVQAGGSLRLSCAASGFTFDDWAIGWFRQAPGKEREGVSCISKRHGTTHYADSVKGRFTISSDNAKNTVYLRMNGLKPEDTAVYYCAASSWGSCTVATMRDVDRYDYDYWGQGTQVTVSS

SEQ ID NO: 11 - protein, 123 aa, synthetic construct [sequence omitted]

SEQ ID NO: 12 - protein, 129 aa, synthetic construct
DVQLVESGGGSVQSGGSLRLSCAASGYTYRRYYMGWFRQAPGEQREGVAVINNDGRTNYADSVKGRFRISRDNAENTLHLEMNSLKPEDTAMYYCAATGNILPPMTAVPPLGRQWYPYWGRGTLVTVSS

SEQ ID NO: 13 - protein, 130 aa, synthetic construct
HVQLVESGGGSVQSGGSLRLSCAASGYAVKNCMGWFRQAPGKEREGVAVINRNGITTYADSVKGRFTISQDKDKNTLDLQMNSLKPEDTAMYYCAATPTLLTIPARFLCDVRNPSGFTDWGQGTLVTVSS

SEQ ID NO: 14 - protein, 128 aa, synthetic construct
QVQLVESGGGSVQAGGSLRLSCVVSAYSAYTYKTMCMGWFRQAPGKEREGVAAIYRGGLNTYYADSVKGRFIISRDNAESTMYLQMNSLKPEDTAMYYCAADWLRGDDCNIGANFDYWGQGTQVTVSS

SEQ ID NO: 15 - protein, 132 aa, synthetic construct
QVQLVESGGGSVQAGGSLRLSCVATGFTISRKCMGWFREAPGKKREVIATINTGSSSPYYADGVKGRFTISQDNAKNTVYLQMNSLKPEDTAMYYCAATKGVVVGTGYCGGPYVERPNSAYWGQGTQVTVSS

SEQ ID NO: 16 - protein, 126 aa, synthetic construct
DVQLVESGGGSVQAGRSLRLSCELSDYTWSTVCMGWFRQAPGKEREGVAVIYTRSGGTTYADSAKGRFTISRDNAKDTLYLQMDSLKPEDTAMYYCAAGPLYDGRCTYRSPAFHYWGQGTQVTVSS

SEQ ID NO: 17 - protein, 122 aa, synthetic construct
DVQLVESGGGSAQAGGSLRLSCAASGPTSSLRTMGWFRQASGKERERVAVIWDGRTTDYDDSVQDRFTISQDNAKSTVYLQMNTLKPEDTAMYYCAASPRIVPFASTYFQHWGQGTQVTVSS

SEQ ID NO: 18 - protein, 127 aa, synthetic construct
HVQLVESGGGSVQAGGSLKLSCAASGSIFSGSIFSRCGMRWYRQAPGKERELVSSTSKDGFTSYTDSVKGRFTISQDNANNTLYLQMSSLKTEDTAVYSCAAICAVGGYSLSTYTYWGQGTQVTVSS

SEQ ID NO: 19 - protein, 124 aa, synthetic construct
EVQLVESGGDSVQAGGSLRLSCAASGYSPGSYCMGWFRQAPGKERERVAIIESRGTVTYVDSVKGRFTISKDNAKNTLYLQMNSLKPEDTAMYYCAASRPWSGVRCLHDKYDYWGQGTQVTVSS

SEQ ID NO: 20 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 22 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 23 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 27 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 33 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 35 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 37 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 39 - protein, 129 aa, synthetic construct
HVQLVESGGGSVQSGGSLRLSCAVSGYAYSSLAWFRQAPGKEREGVAALLTAIGGPTRTTYADSVKGRLAISQDHAKNTLYLQMSSLKPEDTAMYYCAAGRPAGTPRWLLLAPRDYNYWGQGTQVTVSS

SEQ ID NO: 40 - protein, 115 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 41 - protein, 117 aa, synthetic construct
QVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSS

SEQ ID NO: 42 - protein, 127 aa, synthetic construct
QVQLVQSGGGLVQAGGSLSLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSS

SEQ ID NO: 43 - protein, 116 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 44 - protein, 117 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 45 - protein, 120 aa, synthetic construct
QVQLQESGGGLVQAGGSLRLSCAASGRTVSSLFMGWFRQAPGKERELVAAISRYSLYTYYADSVKGRFTISADNAKNAVYLQMNSLKPEDTAVYYCASKLEYTSNDYDSWGQGTQVTVSS

SEQ ID NO: 46 - protein, 120 aa, synthetic construct
QVQLQESGGGLVQAGDSLRLSCAASGRAVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 47 - protein, 120 aa, synthetic construct
QVQLQESGGGLVQAGDSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 48 - protein, 116 aa, synthetic construct
QVQLQESGGGLVQPGDSMRLSCAAEGDSLDGYVVGWFRQAPGKERQGVSSISGDGSMRYVADSVKGRFTISRDNAKNTVYLQMIDLKPEDTGVYYCAADPPTWDYWGQGTQVTVSS

SEQ ID NO: 49 - protein, 120 aa, synthetic construct
QVQLQESGGGLVQPGGSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 50 - protein, 120 aa, synthetic construct
QVQLQESGGGLVQAGESLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 51 - protein, 111 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 52 - protein, 125 aa, synthetic construct
QLQLVESGGGLVHAGGSLRLSCAASGSTFSINAIGWYRQAPGKQRELVAALSSGGSKNYADSVKGRFTISRDNAKNTVYLQMNRLKPEDTAVYYCNAEIYYSDGVDDGYRGMDYWGKGTQVTVSS

SEQ ID NO: 53 - protein, 129 aa, synthetic construct
EVQVVESGGGLVQTGGSLRLSCAASGPPLSSYAVAWFRQTPGKEREFVAAISWSGSNTYYADSVKGRFTISKDNAKNTVLVYLQMNSLKPEDTAVYYCAADRRGGPLSDYEWEDEYADWGQGTQVTVSS

SEQ ID NO: 54 - protein, 9 aa, Homo sapiens
LVVVGAVGV

SEQ ID NO: 55 - protein, 10 aa, Homo sapiens
KLVVVGADGV

SEQ ID NO: 56 - protein, 10 aa, Homo sapiens
KLVVVGAVGV

SEQ ID NO: 57 - protein, 9 aa, Homo sapiens
GADGVGKSA

SEQ ID NO: 58 - protein, 10 aa, Homo sapiens
GADGVGKSAL

SEQ ID NO: 59 - protein, 9 aa, Homo sapiens
VVGADGVGK

SEQ ID NO: 60 - protein, 9 aa, Homo sapiens
VVGAVGVGK

SEQ ID NO: 61 - protein, 10 aa, Homo sapiens
VVVGADGVGK

SEQ ID NO: 62 - protein, 10 aa, Homo sapiens
VVVGAVGVGK

SEQ ID NO: 63 - protein, 9 aa, Homo sapiens
EVDPIGHLY

SEQ ID NO: 64 - protein, 9 aa, Homo sapiens
FLWGPRALV

SEQ ID NO: 65 - protein, 9 aa, Homo sapiens
ALSRKVAEL

SEQ ID NO: 66 - protein, 9 aa, Homo sapiens
KVTDLVQFL

SEQ ID NO: 67 - protein, 9 aa, Homo sapiens
GLYDGMEHL

SEQ ID NO: 68 - protein, 9 aa, Homo sapiens
SLLKFLAKV

SEQ ID NO: 69 - protein, 9 aa, Homo sapiens
ALLPAVPSL

SEQ ID NO: 70 - protein, 9 aa, Homo sapiens
RLDQLLRHV

SEQ ID NO: 71 - protein, 9 aa, Homo sapiens
SLLQHLIGL

SEQ ID NO: 72 - protein, 11 aa, Homo sapiens
SLFHPEDTGQV

SEQ ID NO: 73 - protein, 11 aa, Homo sapiens
SIEPEEFLTPK

SEQ ID NO: 74 - protein, 9 aa, Homo sapiens
SLYTKVVHY

SEQ ID NO: 75 - protein, 9 aa, Homo sapiens
FMVFLQTHI

SEQ ID NO: 76 - protein, 9 aa, Homo sapiens
CLGGLLTMV

SEQ ID NO: 77 - protein, 9 aa, Homo sapiens
ALYDKTKRI

SEQ ID NO: 78 - protein, 9 aa, Homo sapiens
KLFTSVFGV

SEQ ID NO: 79 - protein, 9 aa, Homo sapiens
SLWRRPFNV

SEQ ID NO: 80 - protein, 9 aa, Homo sapiens
GLMATPAKL

SEQ ID NO: 81 - protein, 9 aa, Homo sapiens
RLFEQVMRI

SEQ ID NO: 82 - protein, 11 aa, Homo sapiens
ATYKEVSKMVK

SEQ ID NO: 83 - protein, 9 aa, Homo sapiens
LYAPTILLW

SEQ ID NO: 84 - protein, 9 aa, Homo sapiens
KIQEILTQV

SEQ ID NO: 85 - protein, 9 aa, Homo sapiens
GYLQGLVSF

SEQ ID NO: 86 - protein, 9 aa, Homo sapiens
HPEYNRPLL

SEQ ID NO: 87 - protein, 9 aa, Homo sapiens
TLMSAMTNL

SEQ ID NO: 88 - protein, 9 aa, Homo sapiens
HPEDTGQVF

SEQ ID NO: 89 - protein, 9 aa, Homo sapiens
FLDKGTYTL

SEQ ID NO: 90 - protein, 9 aa, Homo sapiens
YLSGANLNL

SEQ ID NO: 91 - protein, 9 aa, Homo sapiens
KIFGSLAFL

SEQ ID NO: 92 - protein, 9 aa, Homo sapiens
YLLEMLWRL

SEQ ID NO: 93 - protein, 9 aa, Homo sapiens
KVAELVHFL

SEQ ID NO: 94 - protein, 10 aa, Homo sapiens
GVYDGREHTV

SEQ ID NO: 95 - protein, 9 aa, Homo sapiens
KVLEHVVRV

SEQ ID NO: 96 - protein, 9 aa, Homo sapiens
SLLMWITQV

SEQ ID NO: 97 - protein, 9 aa, Homo sapiens
SLLMWITQA

SEQ ID NO: 98 - protein, 9 aa, Homo sapiens
LLGRNSFEV

SEQ ID NO: 99 - protein, 9 aa, Homo sapiens
LLHETDSAV

SEQ ID NO: 100 - protein, 9 aa, Homo sapiens
LTLGEFLKL

SEQ ID NO: 101 - protein, 9 aa, Homo sapiens
YMDGTMSQV

SEQ ID NO: 102 - protein, 9 aa, Homo sapiens
RMFPNAPYL

SEQ ID NO: 103 - protein, 9 aa, Homo sapiens
SLYNTVATL

SEQ ID NO: 104 - protein, 9 aa, Homo sapiens
ILKEPVHGV

SEQ ID NO: 105 - protein, 9 aa, Homo sapiens
AISPRTLNA

SEQ ID NO: 106 - protein, 9 aa, Homo sapiens
FLLTRILTI

SEQ ID NO: 107 - protein, 11 aa, Homo sapiens
GTLPQDHIVQK

SEQ ID NO: 108 - protein, 10 aa, Homo sapiens
FLPSDFFPSV

SEQ ID NO: 109 - protein, 10 aa, Homo sapiens
YMLDLQPETT

SEQ ID NO: 110 - protein, 9 aa, Homo sapiens
YMLDLQPET

SEQ ID NO: 111 - protein, 9 aa, Homo sapiens
LLMGTLGIV

SEQ ID NO: 112 - protein, 11 aa, synthetic construct
  VARIANT 1..10: note = This region may encompass 1-10 residues
GGGGGGGGGGS

SEQ ID NO: 113 - protein, 11 aa, synthetic construct
  VARIANT 2..11: note = This region may encompass 1-10 residues
SGGGGGGGGGG

SEQ ID NO: 114 - protein, 5 aa, synthetic construct
GGGGS

SEQ ID NO: 115 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 116 - protein, 10 aa, synthetic construct
GGGGSGGGGS

SEQ ID NO: 117 - protein, 20 aa, synthetic construct
GGGGSGGGGSGGGGSGGGGS

SEQ ID NO: 118 - protein, 25 aa, synthetic construct
GGGGSGGGGSGGGGSGGGGSGGGGS

SEQ ID NO: 119 - protein, 4 aa, synthetic construct
GGGG

SEQ ID NO: 120 - protein, 5 aa, synthetic construct
GGGGG

SEQ ID NO: 121 - protein, 6 aa, synthetic construct
GGGGGG

SEQ ID NO: 122 - protein, 7 aa, synthetic construct
GGGGGGG

SEQ ID NO: 123 - protein, 8 aa, synthetic construct
GGGGGGGG

SEQ ID NO: 124 - protein, 9 aa, synthetic construct
GGGGGGGGG

SEQ ID NO: 125 - protein, 4 aa, synthetic construct
GGGS

SEQ ID NO: 126 - protein, 5 aa, synthetic construct
GSGGS

SEQ ID NO: 127 - protein, 5 aa, synthetic construct
AAAGG

SEQ ID NO: 128 - protein, 5 aa, synthetic construct
ADAAP

SEQ ID NO: 129 - protein, 11 aa, synthetic construct
ADAAPTVSIFP

SEQ ID NO: 130 - protein, 12 aa, synthetic construct
ADAAPTVSIFPP

SEQ ID NO: 131 - protein, 6 aa, synthetic construct
AKTTAP

SEQ ID NO: 132 - protein, 13 aa, synthetic construct
AKTTAPSVYPLAP

SEQ ID NO: 133 - protein, 17 aa, synthetic construct
AKTTPKLEEGEFSEARV

SEQ ID NO: 134 - protein, 9 aa, synthetic construct
AKTTPKLGG

SEQ ID NO: 135 - protein, 6 aa, synthetic construct
AKTTPP

SEQ ID NO: 136 - protein, 13 aa, synthetic construct
AKTTPPSVTPLAP

SEQ ID NO: 137 - protein, 6 aa, synthetic construct
ASTKGP

SEQ ID NO: 138 - protein, 26 aa, synthetic construct
ASTKGPSVFPLAPASTKGPSVFPLAP

SEQ ID NO: 139 - protein, 14 aa, synthetic construct
EGKSSGSGSESKST

SEQ ID NO: 140 - protein, 15 aa, synthetic construct
GEGESGEGESGEGES

SEQ ID NO: 141 - protein, 20 aa, synthetic construct
GEGESGEGESGEGESGEGES

SEQ ID NO: 142 - protein, 15 aa, synthetic construct
GEGGSGEGGSGEGGS

SEQ ID NO: 143 - protein, 15 aa, synthetic construct
GENKVEYAPALMALS

SEQ ID NO: 144 - protein, 15 aa, synthetic construct
GGEGSGGEGSGGEGS

SEQ ID NO: 145 - protein, 15 aa, synthetic construct
GGGESGGEGSGEGGS

SEQ ID NO: 146 - protein, 15 aa, synthetic construct
GGGESGGGESGGGES

SEQ ID NO: 147 - protein, 15 aa, synthetic construct
GGGKSGGGKSGGGKS

SEQ ID NO: 148 - protein, 15 aa, synthetic construct
GGGKSGGKGSGKGGS

SEQ ID NO: 149 - protein, 5 aa, synthetic construct
GGGSG

SEQ ID NO: 150 - protein, 15 aa, synthetic construct
GGKGSGGKGSGGKGS

SEQ ID NO: 151 - protein, 4 aa, synthetic construct
GGSG

SEQ ID NO: 152 - protein, 5 aa, synthetic construct
GGSGG

SEQ ID NO: 153 - protein, 9 aa, synthetic construct
GGSGGGGSG

SEQ ID NO: 154 - protein, 13 aa, synthetic construct
GGSGGGGSGGGGS

SEQ ID NO: 155 - protein, 15 aa, synthetic construct
GHEAAAVMQVQYPAS

SEQ ID NO: 156 - protein, 15 aa, synthetic construct
GKGGSGKGGSGKGGS

SEQ ID NO: 157 - protein, 15 aa, synthetic construct
GKGKSGKGKSGKGKS

SEQ ID NO: 158 - protein, 20 aa, synthetic construct
GKGKSGKGKSGKGKSGKGKS

SEQ ID NO: 159 - protein, 15 aa, synthetic construct
GKPGSGKPGSGKPGS

SEQ ID NO: 160 - protein, 21 aa, synthetic construct
GKPGSGKPGSGKPGSGKPSGS

SEQ ID NO: 161 - protein, 15 aa, synthetic construct
GPAKELTPLKEAKVS

SEQ ID NO: 162 - protein, 12 aa, synthetic construct
GSAGSAAGSGEF

SEQ ID NO: 163 - protein, 5 aa, synthetic construct
GSGGG

SEQ ID NO: 164 - protein, 5 aa, synthetic construct
GSGSG

SEQ ID NO: 165 - protein, 4 aa, synthetic construct
GSSG

SEQ ID NO: 166 - protein, 10 aa, synthetic construct
GSSGGSGGSG

SEQ ID NO: 167 - protein, 11 aa, synthetic construct
GSSGGSGGSGG

SEQ ID NO: 168 - protein, 12 aa, synthetic construct
GSSGGSGGSGGS

SEQ ID NO: 169 - protein, 13 aa, synthetic construct
GSSGGSGGSGGSG

SEQ ID NO: 170 - protein, 16 aa, synthetic construct
GSSGGSGGSGGSGGGS

SEQ ID NO: 171 - protein, 11 aa, synthetic construct
GSSGGSGGSGS

SEQ ID NO: 172 - protein, 5 aa, synthetic construct
GSSGT

SEQ ID NO: 173 - protein, 5 aa, synthetic construct
GSSSG

SEQ ID NO: 174 - protein, 18 aa, synthetic construct
GSTSGSGKPGSGEGSTKG

SEQ ID NO: 175 - protein, 19 aa, synthetic construct
GTAAAGAGAAGGAAAGAAG

SEQ ID NO: 176 - protein, 19 aa, synthetic construct
GTSGSSGSGSGGSGSGGGG

SEQ ID NO: 177 - protein, 14 aa, synthetic construct
IRPRAIGGSKPRVA

SEQ ID NO: 178 - protein, 18 aa, synthetic construct
KESGSVSSEQLAQFRSLD

SEQ ID NO: 179 - protein, 15 aa, synthetic construct
KTTPKLEEGEFSEAR

SEQ ID NO: 180 - protein, 19 aa, synthetic construct
PRGASKSGSASQTGSAPGS

SEQ ID NO: 181 - protein, 6 aa, synthetic construct
QPKAAP

SEQ ID NO: 182 - protein, 13 aa, synthetic construct
QPKAAPSVTLFPP

SEQ ID NO: 183 - protein, 27 aa, synthetic construct
RADAAAAGGGGSGGGGSGGGGSGGGGS

SEQ ID NO: 184 - protein, 12 aa, synthetic construct
RADAAAAGGPGS

SEQ ID NO: 185 - protein, 6 aa, synthetic construct
RADAAP

SEQ ID NO: 186 - protein, 9 aa, synthetic construct
RADAAPTVS

SEQ ID NO: 187 - protein, 6 aa, synthetic construct
SAKTTP

SEQ ID NO: 188 - protein, 18 aa, synthetic construct
SAKTTPKLEEGEFSEARV

SEQ ID NO: 189 - protein, 10 aa, synthetic construct
SAKTTPKLGG

SEQ ID NO: 190 - protein, 14 aa, synthetic construct
STAGDTHLGGEDFD

SEQ ID NO: 191 - protein, 5 aa, synthetic construct
TVAAP

SEQ ID NO: 192 - protein, 12 aa, synthetic construct
TVAAPSVFIFPP

SEQ ID NO: 193 - protein, 24 aa, synthetic construct
TVAAPSVFIFPPTVAAPSVFIFPP

SEQ ID NO: 194 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 197 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 198 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 199 - protein, 4 aa, synthetic construct
CPPC

SEQ ID NO: 200 - protein, 4 aa, Homo sapiens
CPSC

SEQ ID NO: 201 - protein, 20 aa, synthetic construct
EPKSCDKTHTCPPCPAPPVA

SEQ ID NO: 202 - protein, 17 aa, synthetic construct
ESKYGPPCPPCPAPPVA

SEQ ID NO: 203 - protein, 15 aa, synthetic construct
CPPCPAPGGGGPSVF

SEQ ID NO: 204 - protein, 14 aa, synthetic construct
CPPCPAPGGGPSVF

SEQ ID NO: 205 - protein, 13 aa, synthetic construct
CPPCPAPGGPSVF

SEQ ID NO: 206 - protein, 12 aa, synthetic construct
CPPCPAPGPSVF

SEQ ID NO: 207 - protein, 108 aa, synthetic construct; identical to SEQ ID NO: 3

(19) United States
(12) Patent Application Publication
(10) Pub. No.: US 2025/0376524 A1
(43) Pub. Date: Dec. 11, 2025

(54) TETRAVALENT MULTISPECIFIC BINDING MOLECULES AND METHODS OF USE THEREOF

(71) Applicant: Regeneron Pharmaceuticals, Inc., Tarrytown, NY (US)

(72) Inventors: Lauric HABER, Rye Brook, NY (US); Jennifer FINNEY, Park Ridge, NJ (US); Ryan MCKAY, Somers, NY (US); Eric SMITH, New York, NY (US); Chia-Yang LIN, Scarsdale, NY (US)

(73) Assignee: Regeneron Pharmaceuticals, Inc., Tarrytown, NY (US)

(21) Appl. No.: 19/230,703

(22) Filed: Jun. 6, 2025

Related U.S. Application Data
(60) Provisional application No. 63/657,387, filed on Jun. 7, 2024.

(51) Int. Cl. C07K 16/28; C07K 16/30

(52) U.S. Cl. CPC C07K 16/2809; C07K 16/2833; C07K 16/30; C07K 2317/31; C07K 2317/52; C07K 2317/55; C07K 2317/622; C07K 2317/73

(57) ABSTRACT

The present disclosure relates to multispecific binding molecules comprising a polypeptide chain comprising, in N- to C-terminal orientation, an antigen binding domain (e.g., an scFv, a sdAb), a dimerization moiety (e.g., an Fc domain), and a Fab component (e.g., VH or VL). Certain aspects relate to multimeric molecules comprising two polypeptide chains, each comprising, in N- to C-terminal orientation, an antigen binding domain (e.g., an scFv, a sdAb), a dimerization moiety (e.g., an Fc domain), and a Fab component (e.g., VH or VL). The multispecific binding molecules may further comprise one or more additional polypeptide chains associated with the Fab component to form a Fab. The disclosure further provides pharmaceutical compositions comprising the multispecific binding molecules, and methods of use of the multispecific binding molecules in antigen-specific immune activation and in therapeutic applications, as well as nucleic acids encoding the multispecific binding molecules, recombinant cells that express the multispecific binding molecules, and methods of producing the multispecific binding molecules.

### 1. CROSS-REFERENCE TO RELATED APPLICATIONS

This application claims the priority benefit of U.S. provisional application No. 63/657,387, filed on Jun. 7, 2024, the contents of which are incorporated herein in their entirety by reference thereto.

### 2. SEQUENCE LISTING

The instant application contains a Sequence Listing which has been submitted electronically and is hereby incorporated by reference in its entirety. Said copy, created on Jun. 3, 2025, is named RGN-051US_SL.xml and is 191,901 bytes in size.

### 3. BACKGROUND

Numerous biological therapeutics have been developed for the prevention and/or treatment of diseases including but not limited to proliferative diseases (e.g., cancers), infectious diseases (e.g., viral, bacterial, fungal, or protozoal infections), and inflammatory diseases (e.g., Crohn's disease). Multispecific antigen-binding molecules that bind to more than one target antigen are particularly effective in a number of therapeutic contexts. For example, molecules that bind to both a target antigen (e.g. a cancerous antigen or an infectious antigen) and an immune cell antigen (e.g., a T cell antigen) are useful for triggering an immune response against the ailment associated with the target antigen, often with the goal of selective destruction of the cells expressing the target antigen.

One use for multispecific antibodies is the activation of immune cells (e.g., T cells) by an antibody containing one arm targeting a cancer cell (e.g., tumor-associated antigens, TAAs) and another arm targeting a T cell (e.g., CD3). However, the task of generating multispecific molecules suitable for treatment provides several technical challenges related to toxicity, as TAAs are typically expressed on normal cells as well as tumor cells. Antigens specifically expressed by tumor cells are optimal for these therapeutics, however such tumor-specific antigens are often present at a low density on the cancer cells, for example by virtue of being expressed as HLA-bound peptides, posing difficulties for effective targeting.

Alternative binding molecule formats hold promise for targeting multiple antigens with high specificity and selectivity. There is a need for new multispecific antigen-binding molecule formats to increase the repertoire of available binding molecules, for example to identify those that will trigger an immune response with high specificity and efficacy even for low copy number target antigens.

### 4. SUMMARY

The present disclosure relates to multispecific binding molecules (MBMs) comprising two polypeptides, each comprising, in N- to C-terminal orientation, an antigen binding domain (e.g., an scFv or sdAb); a first dimerization moiety (e.g., an Fc domain); and a portion of a first Fab (e.g., a VH domain or VL domain). As disclosed herein, it was surprisingly found that this MBM configuration significantly decreased immunogenicity relative to other formats. In some embodiments, the first antigen binding domain and the first Fab bind to a first target, while the second antigen binding domain and the second Fab bind to a second target. In some embodiments, one antigen binding domain (e.g., scFv) and Fab are T cell antigen (TCA) targeting moieties, while the other antigen binding domain (e.g., scFv) and Fab target a different molecule (e.g., a tumor antigen, an infectious disease antigen); in such embodiments, as described herein, it was surprisingly found that this MBM format significantly enhanced T cell activation relative to other formats, even when using the same antigen binding sequences (e.g., VH and VL). MBMs described herein are useful in methods of simultaneous targeting of two or more targets, including various therapeutic and prophylactic methods. For example, an MBM of the disclosure comprising one or more TCA targeting moieties are useful in methods where stimulation of the immune system of a host is beneficial, including, for example, in treatment of proliferative disorders such as cancer.

Exemplary targeting moieties that can be used in the MBMs of the disclosure are described in Section 6.4, and include T cell antigen (TCA) targeting moieties (e.g., as described in Section 6.4.1), tumor antigen targeting moieties (e.g., as described in Section 6.4.1.1.2 and subsections thereof), low-density antigen targeting moieties (Section 6.4.3), and HLA-bound peptide antigen targeting moieties (Section 6.4.4).

In some cases, MBMs disclosed herein comprise a multimerization moiety such as an Fc region. Exemplary Fc regions that can be used in the MBMs of the disclosure are described in Section 6.6, including Fc regions comprising Fc domains with altered effector function (Section 6.6.1.1) and Fc domains that confer heterodimerization capability to the MBM (Section 6.6.1.2).

Linkers that can be used to connect different components of the MBMs of the disclosure are described in Section 6.5.

Various exemplary configurations of the MBMs of the disclosure are described in specific embodiments 1 to 164, infra.

The disclosure further provides nucleic acids encoding the MBMs of the disclosure. The nucleic acids encoding the MBMs can be a single nucleic acid (e.g., a vector encoding all polypeptide chains of an MBMs) or a plurality of nucleic acids (e.g., two or more vectors encoding the different polypeptide chains of an MBMs). The disclosure further provides host cells and cell lines engineered to express the nucleic acids and MBMs of the disclosure. The disclosure further provides methods of producing an MBM of the disclosure. Exemplary nucleic acids, host cells, and cell lines, and methods of producing an MBM are described in Section 6.7 and specific embodiments 165 to 168, infra.

The disclosure further provides pharmaceutical compositions comprising the MBMs of the disclosure. Exemplary pharmaceutical compositions are described in Section 6.8 and specific embodiments 172 to 179, infra.

Further provided herein are methods of using the MBMs and the pharmaceutical compositions of the disclosure, e.g., for treating or preventing cancer. Exemplary methods are described in 6.9 and include therapeutic methods and prophylactic methods. Specific embodiments of the methods of treatment of the disclosure are described in specific embodiments 180 to 193, infra.

### 5. BRIEF DESCRIPTION OF THE FIGURES

FIGS. 1A-1C show exemplary structures of multispecific binding molecules (MBMs) of the disclosure. FIG. 1A shows the structure of an MBM comprising two half antibodies, each comprising, from N- to C-terminus, an scFv, an Fc domain, and a Fab. FIG. 1B shows the structure of an MBM comprising: (a) a first half antibody comprising, from N- to C-terminus, a single domain antibody (sdAb), designated “VHH”, an Fc domain, and a Fab; and (b) a second half antibody comprising, from N- to C-terminus, an scFv, an Fc domain, and a Fab. FIG. 1C shows the structure of an MBM comprising two half-antibodies, each comprising, from N- to C-terminus, a single domain antibody (sdAb), designated “VHH”, an Fc domain, and a Fab. MBMs of the disclosure include homodimeric MBMs (comprising two identical half-antibodies) as well as heterodimeric MBMs (comprising two different half-antibodies, e.g., each having targeting domains that bind to different epitopes and/or target antigens). The Fab may be any Fab (e.g., as described in Section 6.3.2). Although depicted as composed of two separate polypeptides chains, in some cases, the Fab is a single chain Fab. Although not depicted, asymmetrical constructs may include one or more Fc heterodimerization variants (e.g., as described in Section 6.6.1.2), such as knob and hole mutations and/or “star” mutations.

FIG. 2 is a graph showing the percent viability of A375 cells treated with the depicted binding molecule over a range of concentrations.

FIGS. 3A-3C demonstrate differences in potency of certain MBMs against wild-type A375 cells and “Clone 8” A375 cells. FIG. 3A displays the exemplary MBM structures and descriptions associated with the symbols used in FIGS. 3B and 3C. FIG. 3B is a graph showing the percent viability of wild-type A375 cells treated with different concentrations of MBM. FIG. 3C is a graph showing the percent viability of “Clone 8” A375 cells treated with different concentrations of MBM.

FIG. 4 shows the reactivity to pre-existing anti-drug antibodies (ADA) associated with the depicted binding molecules.

FIGS. 5A and 5B show differences in potency of various binding molecules against wild-type A375 cells. FIG. 5A displays the binding molecule structures and descriptions associated with the symbols used in FIG. 5B. FIG. 5B is a graph showing the percent viability of wild-type A375 cells treated with different concentrations of the indicated binding molecule.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding Domain: The term “antigen-binding domain” or “ABD” as used herein refers to the portion of a targeting moiety that is capable of specific, non-covalent, and reversible binding to a target molecule.

Associated: The term “associated” in the context of a multispecific binding molecule refers to a functional relationship between two or more polypeptide chains or portions of a polypeptide chain. In particular, the term “associated” means that two or more polypeptides are associated with one another, e.g., non-covalently through molecular interactions or covalently through one or more disulfide bridges or chemical cross-linkages, so as to produce a functional multispecific binding molecule. Examples of associations that might be present in a multispecific binding molecule of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Bivalent: The term “bivalent” as used herein in the context of a multispecific binding molecule (MBM) refers to an MBM that has two antigen-binding domains. The domains can be the same or different. Accordingly, a bivalent antigen-binding molecule can be monospecific or bispecific.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABD definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABD numbering scheme); and Lefranc et al., 2003, Dev. Comp. Immunol. 27:55-77 (IMGT numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align. Public databases are available for identifying CDR sequences within an antibody.

Dimerization Moiety: The term “dimerization moiety” refers to a polypeptide chain or an amino acid sequence capable of facilitating an association between two polypeptide chains to form a dimer. A first dimerization moiety can associate with an identical second dimerization moiety, or can associate with a second dimerization moiety that is different from the first. In some embodiments, a dimerization moiety is an Fc domain, with the association of two Fc domains forming an Fc region. Thus, the Fc region can be homodimeric or heterodimeric.

EC50: The term “EC50” refers to the half maximal effective concentration of a molecule, such as a multispecific binding molecule, which induces a response halfway between the baseline and maximum after a specified exposure time. The EC50 essentially represents the concentration of a molecule where 50% of its maximal effect is observed. Thus, reduced or weaker binding is observed with an increased EC50, or half maximal effective concentration value.

Epitope: An epitope (or “antigenic determinant”) is a portion of an antigen (e.g., polypeptide antigen) recognized by an antibody or other antigen-binding moiety as described herein. An epitope can be linear or conformational.

Fab: The term “Fab” in the context of a multispecific binding molecule of the disclosure of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab (a type of “domain exchanged” arrangement). Alternatively, or in addition to, the use of substituted or swapped constant domains, correct chain pairing can be achieved by the use of universal light chains that can pair with both variable regions of a heterodimeric multispecific binding molecule of the disclosure. The term “Fab” encompasses single chain Fabs.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction and/or for purification, e.g., via star mutations.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Half Antibody: The term “half antibody” refers to a molecule that comprises at least one Fc domain and can associate with another molecule comprising an Fc through, e.g., a disulfide bridge or molecular interactions. A half antibody can be composed of one polypeptide chain or more than one polypeptide chains (e.g., the two polypeptide chains of a Fab). An example of a half antibody is a molecule comprising a heavy and light chain of an antibody (e.g., an IgG antibody). An example of a half antibody is a molecule comprising a first polypeptide comprising an scFv, a CH2 domain, a CH3 domain, a VH domain, and a CH1 domain, and a second polypeptide comprising a VL domain and a CL domain, where the VL and VH domains associate to form a Fab.

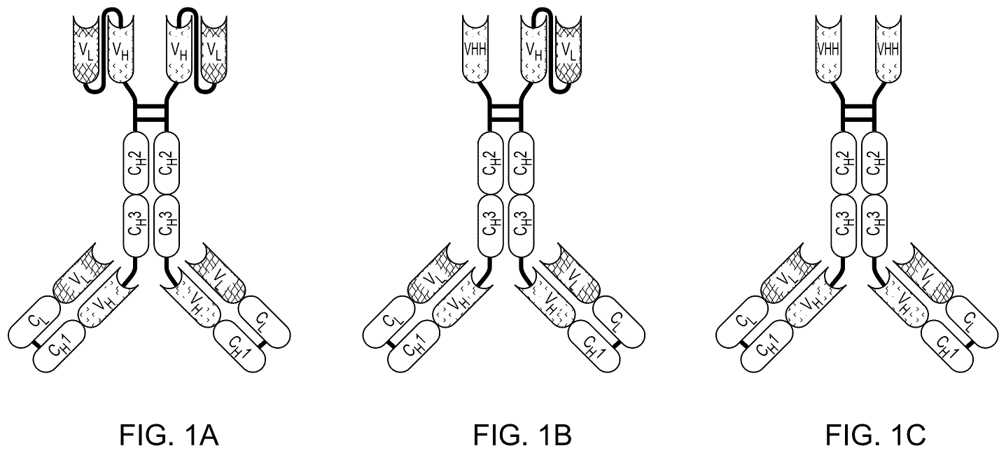

In some embodiments, a half antibody comprises a) a first polypeptide chain comprising (in N- to C-terminal order) an scFv (e.g., VL-linker-VH or VH-linker-VL), a CH2 domain, a CH3 domain, a VH domain, and a CH1 domain, and b) a second polypeptide chain comprising (in N- to C-terminal order) a VL domain and a CL domain (as depicted in, e.g., FIG. 1A, both half antibodies, and in FIG. 1B, right half antibody). Alternatively, the first polypeptide chain may comprise a VL domain in place of the VH domain, while the second polypeptide chain comprises a VH domain in place of the VL domain. This half antibody is referred herein to as a “Type 1” half antibody for convenience.

In some embodiments, a half antibody comprises a) a first polypeptide chain comprising (in N- to C-terminal order) a sdAb, a CH2 domain, a CH3 domain, a VH domain, and a CH1 domain, and b) a second polypeptide chain comprising (in N- to C-terminal order) a VL domain and a CL domain (as depicted in, e.g., FIG. 1B, left half antibody, and in FIG. 1C, both half antibodies). Alternatively, the first polypeptide chain may comprise a VL domain in place of the VH domain, while the second polypeptide chain comprises a VH domain in place of the VL domain. This half antibody is referred herein to as a “Type 2” half antibody for convenience.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host Cell or Recombinant Host Cell: The terms “host cell” and “recombinant host cell” as used herein refer to a cell that has been genetically engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a multispecific binding molecule, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Immune Response: The term “immune response” refers to an integrated bodily response to an antigen and preferably refers to a cellular immune response or a cellular as well as a humoral immune response. The immune response may be protective (also “preventive” or “prophylactic”) and/or therapeutic. The terms “inducing an immune response,” “eliciting an immune response,” and the like, as used herein, can indicate that there was no immune response against a particular antigen before administration of a particular composition (e.g., an MBM of the disclosure), but it may also indicate that there was a certain level of immune response against a particular antigen before such administration, and that after induction the immune response is enhanced. Thus, “inducing an immune response” also includes “enhancing an immune response”. Preferably, after inducing an immune response in a subject, said subject is protected from developing a disease (e.g., cancer) or the disease condition is ameliorated (e.g., tumor reduction, reduction in cancer cell number, etc.) by inducing an immune response. For example, an immune response against a tumor antigen may be induced in a patient having cancer or in a subject at risk of developing a cancer. Inducing an immune response in this case may mean that the disease condition of the subject is ameliorated, that the subject does not develop metastases, or that the subject at risk of developing cancer does not develop cancer.

Low-density Antigen: The term “low-density antigen” as used herein refers to a polypeptide antigen present on the surface of a target cell or population of target cells (either as a full-length polypeptide or presented as an HLA-bound peptide) at an average of no more than 5000 copies per cell. In some embodiments, a low-density antigen is present at an average of no more than 4000, no more than 3000, no more than 2000, no more than 1000, no more than 900, no more than 800, no more than 700, no more than 600, no more than 500, no more than 400, no more than 300, no more than 200, no more than 100, or no more than 50 copies per cell. Low-density antigens include, for example, full length proteins having at least a portion present on a cell surface as well as HLA-bound peptide antigens.

Major Histocompatibility Complex and MHC: These terms refer to naturally occurring MHC molecules, individual chains of MHC molecules (e.g., MHC class I α (heavy) chain, P2 microglobulin, MHC class II a chain, and MHC class II @chain), individual subunits of such chains of MHC molecules (e.g., α1, α2, and/or α3 subunits of MHC class I α chain, α1-α2 subunits of MHC class II a chain, 31-P2 subunits of MHC class II @chain) as well as portions (e.g., the peptide-binding portions, e.g., the peptide-binding grooves), mutants and various derivatives thereof (including fusions proteins), wherein such portion, mutants and derivatives retain the ability to display an antigenic peptide for recognition by a T cell receptor (TCR), e.g., an antigen-specific TCR. An MHC class I molecule comprises a peptide binding groove formed by the a1 and a2 domains of the heavy chain that can stow a peptide of around 8-10 amino acids. Despite the fact that both classes of MHC bind a core of about 9 amino acids (e.g., 5 to 17 amino acids) within peptides, the open-ended nature of MHC class II peptide binding groove (the a1 domain of a class II MHC a polypeptide in association with the P1 domain of a class II MHC @polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sits directly within the groove at any given time. Conventional identifications of particular MHC variants are used herein. The terms encompass “human leukocyte antigen” or “HLA”. An MHC complex containing a bound antigenic peptide (either as a single polypeptide chain or multiple polypeptide chains) may be referred to herein as an “HLA-bound peptide,” “peptide MHC complex,” or “pMHC”.

Multispecific Binding Molecule: The term “multispecific binding molecule” (also “MBM” or “multispecific antigen-binding molecule”) as used herein refers to a molecule (e.g., assembly of multiple polypeptide chains) comprising two half antibodies and which specifically bind to at least two different epitopes (and in some instances three, four, or more different epitopes). A multispecific binding molecule of the disclosure may be bivalent, trivalent, or otherwise multivalent, and may be monospecific, bispecific, or otherwise multispecific. A multispecific binding molecule of the disclosure may specifically bind to epitopes on one, two, or more different antigens.

Multivalent: The term “multivalent” as used herein refers to a multispecific binding molecule comprising two or more ABDs, on one, two or more polypeptide chains.

Operably Linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence. In the context of a fusion protein or other polypeptide, the term “operably linked” means that two or more amino acid segments are linked so as to produce a functional polypeptide. In the context of a nucleic acid encoding a fusion protein, such as a multispecific binding molecule of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single chain Fv” or “scFv” as used herein refers to a polypeptide chain comprising the VH and VL domains of antibody, where these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315.

Single Domain Antibody or sdAb: The term “single domain antibody” or “sdAb” as used herein refers to an antibody or antigen binding fragment thereof comprising a single binding domain (e.g., heavy chain variable region) capable of binding a target molecule without pairing with a corresponding CDR-containing polypeptide (e.g., a light chain). An sdAb or sdAb fragment can be derived from a VH, a VHH, or from a non-antibody scaffold protein, for example a designed ankyrin repeat protein (darpin), an avimer, an anticalin/lipocalin, a centyrin or a fynomer. A sdAb typically lacks a CH1 domain and thus cannot associate with a light chain.

Single Domain VH Antibody or sdVH: The term “single domain VH” or “sdVH” as used herein refers to a variable region of an sdAb that is not of camelid or cartilaginous fish origin. An sdVH can be, for example, of human or non-human mammalian origin. A basic sdVH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

Specifically (or Selectively) Binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In certain embodiments, the subject is human. Except when noted, the terms “patient” or “subject” are used herein interchangeably.

T Cell Antigen: The term “T cell antigen” (also “TCA”) as used herein refers to any biological molecule (e.g., protein, carbohydrate, lipid or combination thereof) that is present on and/or expressed by a T cell. In some embodiments, at least a portion of a T cell antigen is extracellular (e.g., is a cell surface protein or transmembrane protein having at least one extracellular domain). Particular T cell antigens contemplated herein include, but are not limited to, CD3 and CD28. In some embodiments, the T cell antigen is CD3.

Targeting Moiety: The term “targeting moiety” as used herein refers to any molecule or binding portion thereof that can specifically bind to an antigen. Exemplary targeting moieties include, but are not limited to, antibodies and antigen binding portions thereof (e.g., Fab, scFv, sdAb, etc.). A targeting moiety may be described with reference to the antigen to which it specifically binds. Thus, for example, a “T cell antigen targeting moiety” (or “TCA targeting moiety”) refers to a molecule or binding portion thereof that can specifically bind to a T cell antigen. The TCA targeting moiety can also have a functional activity in addition to binding a T cell antigen. For example, a TCA targeting moiety that is a CD3 targeting moiety (e.g., an anti-CD3 antibody or an antigen binding portion thereof) may facilitate clustering and activation of CD3 on a surface of a T cell, while a TCA targeting moiety that is a CD28 targeting moiety (e.g., an anti-CD28 antibody or an antigen binding portion thereof) may activate CD28 signaling in a T cell. Similarly a “TCR targeting moiety” refers to a molecule or binding portion thereof that can specifically bind to a T cell receptor.

Tetravalent: The term “tetravalent” as used herein refers to a multispecific binding molecule that has four antigen-binding domains. In certain embodiments, all four of the antigen-binding domains bind to the same epitope. In some embodiments, three of the antigen-binding domains bind to the same epitope and the other antigen-binding domain binds to a different epitope, whether of the same target molecule or different target molecules. In other embodiments, two of the antigen-binding domains bind to the same epitope and the other two antigen-binding domains bind to a different epitope, whether of the same target molecule or different target molecules. In further embodiments, all three of the antigen-binding sites domains to different epitopes, whether on the same target molecule or on any combination of two or more different target molecules. Accordingly, a tetravalent multispecific binding molecule may be monospecific, bispecific, trispecific, or tetraspecific.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” (also “tumor associated antigen”) or “TAA” refers to a molecule (typically a protein, carbohydrate, lipid or some combination thereof) that is expressed on the surface of a cancer cell, either entirely or as a fragment (e.g., as a peptide presented by an MHC molecule), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., as a peptide presented by an MHC molecule), and not synthesized or expressed on the surface of a normal cell. Accordingly, the term “TAA” encompasses antigens that are specific to cancer cells, sometimes known in the art as tumor-specific antigens (“TSAs”).

Universal Light Chain, ULC: The term “universal light chain” or “ULC” as used herein in the context of an antigen-biding domain refers to a light chain polypeptide capable of pairing with the heavy chain region of the antigen-biding domain and also capable of pairing with other heavy chain regions. Universal light chains are also known as “common light chains.”

VHH: The term “VHH” refers to a variable region of an antibody consisting of only a heavy chain, e.g., an antibody of camelid or cartilaginous fish origin. A VHH variable region can bind to a target molecule in the absence of a light chain. A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. Multispecific Binding Molecules

The present disclosure provides multispecific binding molecules (MBMs) comprising two polypeptides, each comprising, in N- to C-terminal orientation, an antigen binding domain; a first dimerization moiety; and a portion of a first Fab (e.g., a VH domain or VL domain). MBMs of the disclosure typically comprise (1) a first half antibody comprising a first antigen binding domain on a single polypeptide (e.g., an scFv or sdAb), a first dimerization moiety (e.g., an Fc domain), and a first Fab, and (2) a second half antibody comprising a second antigen binding domain on a single polypeptide (e.g., an scFv or sdAb), a second dimerization moiety (e.g., an Fc domain) that associates with the first dimerization moiety, and a second Fab. Each domain or region of an MBM may optionally be connected via a linker (e.g., as described in Section 6.5).

Accordingly, an MBM of the disclosure generally comprises:
(1) a first half antibody comprising;
   (a) a first antigen binding domain on a single polypeptide, e.g., an scFv or sdAb;
   (b) an optional first linker;
   (c) a first multimerization moiety;
   (d) an optional second linker; and
   (e) a first Fab; and
(2) a second half antibody comprising;
   (a) a second antigen binding domain on a single polypeptide, e.g., an scFv or sdAb;
   (b) an optional third linker;
   (c) a second multimerization moiety;
   (d) an optional fourth linker; and
   (e) a second Fab.

In some embodiments, the first antigen binding domain is an scFv. In some embodiments, the first antigen binding domain is a sdAb (e.g., an sdVH). In some embodiments, the second antigen binding domain is an scFv. In some embodiments, the second antigen binding domain is a sdAb (e.g., an sdVH).

Exemplary targeting moieties suitable for use in the MBMs of the disclosure (e.g., as scFvs, sdAbs, and Fabs) are described in Section 6.4.1.

In some embodiments, the first antigen binding domain and the first Fab specifically bind to the same target. In such cases, the first antigen binding domain and the first Fab may comprise identical VH and/or VL domains. In some embodiments, the second antigen binding domain and the second Fab specifically bind to the same target. In such cases, the second antigen binding domain and the second Fab may comprise identical VH and/or VL domains. In some embodiments, the first antigen binding domain and the first Fab specifically bind to a first target and the second antigen binding domain and the second Fab bind to a second target that is different from that bound by the first antigen binding domain and the first Fab.

In some embodiments, the targeting moiety is a T cell antigen (TCA) targeting moiety, e.g., as described in Section 6.4.1. In particular embodiments, an MBM disclosed herein comprises one or more TCA targeting moieties. In particular embodiments, the MBM comprises two or more TCA targeting moieties. The TCA targeting moieties may all specifically bind to the same T cell antigen (e.g., both of two TCA targeting moieties specifically bind to CD3). In such cases, the TCA targeting moieties may bind to the same epitope (e.g., may have identical ABDs) or may bind to different epitopes (e.g., may have different ABDs). In further embodiments, the TCA targeting moieties bind to different T cell antigens (e.g., one TCA targeting moiety binds to one component of the T cell receptor, while a second binds to a separate component of the T cell receptor).

In some embodiments, the first antigen binding domain (e.g., first scFv or first sdAb) is a TCA targeting moiety (e.g., a CD3 targeting moiety) and the first Fab is a TCA targeting moiety (e.g., a CD3 targeting moiety). In some embodiments, the second antigen binding domain (e.g., second scFv or second sdAb) is a TCA targeting moiety (e.g., a CD3 targeting moiety), and the second Fab is a TCA targeting moiety (e.g., a CD3 targeting moiety).

In some embodiments, the antigen targeting moiety is a tumor antigen targeting moiety, e.g., as described in Section 6.4.1.1.2. The MBM may comprise two or more tumor antigen targeting moieties. In some embodiments, the first antigen binding domain (e.g., first scFv or first sdAb) is a tumor antigen targeting moiety and the first Fab is a tumor antigen targeting moiety. In some embodiments, the second antigen binding domain (e.g., second scFv or second sdAb) is a tumor antigen targeting moiety and the second Fab is a tumor antigen targeting moiety.

In some embodiments, the antigen targeting moiety is a low-density antigen targeting moiety, e.g., as described in Section 6.4.3. The MBM may comprise two or more low-density antigen targeting moieties. In some embodiments, the first antigen binding domain (e.g., first scFv or first sdAb) is a low-density antigen targeting moiety and the first Fab is a low-density antigen targeting moiety. In some embodiments, the second antigen binding domain (e.g., second scFv or second sdAb) is a low-density antigen targeting moiety and the second Fab is a low-density antigen targeting moiety.

In some embodiments, the antigen targeting moiety is an HLA-bound peptide antigen targeting moiety, e.g., as described in Section 6.4.4. The MBM may comprise two or more HLA-bound peptide antigen targeting moieties. In some embodiments, the first antigen binding domain (e.g., first scFv or first sdAb) is an HLA-bound peptide antigen targeting moiety and the first Fab is an HLA-bound peptide antigen targeting moiety. In some embodiments, the second antigen binding domain (e.g., second scFv or second sdAb) is an HLA-bound peptide antigen targeting moiety and the second Fab is an HLA-bound peptide antigen targeting moiety.

In the MBMs of the disclosure, each Fab can be composed of two polypeptide chains, one polypeptide chain bearing the heavy chain variable region and the other polypeptide chain bearing the light chain variable region. Thus, the Fab can comprise heavy and light chain variable domains on separate polypeptide chains. For example, a half antibody comprising the Fab can be composed of two polypeptide chains, where one chain contains the heavy chain variable domain of the Fab, and the other chain comprises the light chain variable domain of the targeting moiety.

In some embodiments, an MBM of the disclosure comprises one or two scFvs (each N-terminal to the dimerization moiety). Each scFv of an MBM of the disclosure comprises a VH domain and VL domain, separated by a linker. The scFv may have the N- to C-terminal orientation VH-linker-VL or VL-linker-VH.

In some embodiments, an MBM of the disclosure comprises one or two sdAbs (each N-terminal to the dimerization moiety).

Various targeting moiety formats are described in more detail in Section 6.3.

The MBM generally contains a multimerization moiety to facilitate multimerization of two half antibodies. Exemplary multimerization moieties include Fc domains, e.g., as described in Section 6.6.1.

The MBM can include one or more linker sequences connecting the various components of the molecule, for example connecting an scFv or sdAb to a multimerization moiety or connecting a multimerization moiety to a portion of a Fab. Exemplary linkers are described in Section 6.5.

Below are some illustrative, non-limiting configurations of the two half antibodies present in MBMs of the disclosure:

Configuration 1 refers to an MBM of the disclosure comprising a configuration as depicted in FIG. 1A. Accordingly, an MBM of the disclosure can comprise:
(a) a first half antibody comprising: i) a first polypeptide comprising (in N- to C-terminal orientation) an scFv, an optional linker, a CH2 domain, a CH3 domain, an optional linker, a VH domain, and a CH1 domain and ii) a second polypeptide comprising (in N- to C-terminal orientation) a VL domain and a CL domain associated with the VH and CH1 domains; and
(b) a second half antibody comprising: i) a first polypeptide comprising (in N- to C-terminal orientation) an scFv, an optional linker, a CH2 domain, a CH3 domain, an optional linker, a VH domain, and a CH1 domain and ii) a second polypeptide comprising (in N- to C-terminal orientation) a VL domain and a CL domain associated with the VH and CH1 domains.

Configuration 2 refers to an MBM of the disclosure comprising a configuration as depicted in FIG. 1B. Accordingly, an MBM of the disclosure can comprise:
(a) a first half antibody comprising: i) a first polypeptide comprising (in N- to C-terminal orientation) a sdAb (e.g., VHH), an optional linker, a CH2 domain, a CH3 domain, an optional linker, a VH domain, and a CH1 domain and ii) a second polypeptide comprising (in N- to C-terminal orientation) a VL domain and a CL domain associated with the VH and CH1 domains; and
(b) a second half antibody comprising: i) a first polypeptide comprising (in N- to C-terminal orientation) an scFv, an optional linker, a CH2 domain, a CH3 domain, an optional linker, a VH domain, and a CH1 domain and ii) a second polypeptide comprising (in N- to C-terminal orientation) a VL domain and a CL domain associated with the VH and CH1 domains.

Configuration 3 refers to an MBM of the disclosure comprising a configuration as depicted in FIG. 1C. Accordingly, an MBM can comprise:
(a) a first half antibody comprising: i) a first polypeptide comprising (in N- to C-terminal orientation) a sdAb (e.g., VHH), an optional linker, a CH2 domain, a CH3 domain, an optional linker, a VH domain, and a CH1 domain and ii) a second polypeptide comprising (in N- to C-terminal orientation) a VL domain and a CL domain associated with the VH and CH1 domains; and
(b) a second half antibody comprising: i) a first polypeptide comprising (in N- to C-terminal orientation) a sdAb (e.g., VHH), an optional linker, a CH2 domain, a CH3 domain, an optional linker, a VH domain, and a CH1 domain and ii) a second polypeptide comprising (in N- to C-terminal orientation) a VL domain and a CL domain associated with the VH and CH1 domains.

### 6.3. Targeting Moiety Formats

In certain aspects, a targeting moiety that can be used in an MBM can be any type of antibody or fragment thereof that retains specific binding to an antigenic determinant. In some embodiments, a targeting moiety is a full-length antibody. In some embodiments a targeting moiety is an immunoglobulin molecule, particularly an IgG class immunoglobulin molecule, more particularly an IgG1 or IgG4 immunoglobulin molecule. In some embodiments, a targeting moiety is an antibody fragment. Antibody fragments include, but are not limited to, VH (or VH) fragments, VL (or VL) fragments, Fab fragments, F(ab′)2 fragments, scFv fragments, Fv fragments, minibodies, diabodies, triabodies, and tetrabodies.

6.3.1. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5 (typically a repeat of a sequence containing the amino acids glycine and serine, such as the amino acid sequence (Gly₄Ser)₃; SEQ ID NO:1), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.3.2. Fab

Fab domains were traditionally produced by proteolytic cleavage of immunoglobulin molecules using enzymes such as papain. In the multispecific binding molecules of the disclosure, the Fab domains are typically recombinantly expressed as part of the multispecific binding molecule.

The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the multispecific binding molecules of the disclosure, particularly when the light chain is not a common or universal light chain, it is advantageous to use Fab heterodimerization strategies to permit the correct association of Fab domains belonging to the same ABD and minimize aberrant pairing of Fab domains belonging to different ABDs. For example, the Fab heterodimerization strategies shown in Table H below can be used:

TABLE H
Fab Heterodimerization Strategies

| STRATEGY | VH | CH1 | VL | CL | REFERENCE |
|---|---|---|---|---|---|
| CrossMabCH1-CL | WT | CL domain | WT | CH1 domain | Schaefer et al., 2011, |
| | | | | | Cancer Cell 2011; |
| | | | | | 20: 472-86; |
| | | | | | PMID: 22014573. |
| orthogonal Fab | 39K, 62E | H172A, | 1R, 38D, | L135Y, | Lewis et al., 2014, |
| VHVRD1CH1CRD2 - | | F174G | (36F) | S176W | Nat Biotechnol |
| VLVRD1CλCRD2 | | | | | 32: 191-8 |
| orthogonal Fab | 39Y | WT | 38R | WT | Lewis et al., 2014, |
| VHVRD2CH1 wt - | | | | | Nat Biotechnol |
| VLVRD2Cλ wt | | | | | 32: 191-8 |
| TCR CαCβ | 39K | TCR Cα | 38D | TCR Cβ | Wu et al., 2015, |
| | | | | | MAbs 7: 364-76 |
| CR3 | WT | T192E | WT | N137K, | Golay at al., 2016, J |
| | | | | S114A | Immunol 196: 3199-211. |
| MUT4 | WT | L143Q, | WT | V133T, | Golay at al., 2016, J |
| | | S188V | | S176V | Immunol 196: 3199-211. |
| DuetMab | WT | F126C | WT | S121C | Mazor et al., 2015, |
| | | | | | MAbs 7: 377-89; |
| | | | | | Mazor et al., 2015, |
| | | | | | MAbs 7: 461-669. |
| Domain | WT | CH3 + knob | WT | CH3 + hole | Wozniak-Knopp et al., |
| exchanged | | or hole | | or knob | 2018, PLoSONE13(4): |
| | | mutation | | mutation | e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of a T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of a multispecific binding molecule of the disclosure. In various embodiments, employing a common light chain as described herein reduces the number of inappropriate species of multispecific binding molecules as compared to employing original cognate VLs. In various embodiments, the VL domains of the multispecific binding molecules are identified from monospecific antibodies comprising a common light chain. In various embodiments, the VH regions of the multispecific binding molecules comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

### 6.3.3. Single Domain Antibodies

In some embodiments, a targeting moiety is a single-domain antibody. A single-domain antibody (sdAb) describes a single antigen-binding domain capable of binding to a cognate antigen. sdAbs are often derived from heavy-chain only antibodies, however they also include single VH domains capable of binding to their cognate antigen in the absence of an associated light chain. In some embodiments, sdAbs also include single VL domains capable of binding to their cognate antigen in the absence of an associated light chain. Single VH or VL domains may have amino acid changes relative to native VH or VL sequences that stabilize the domains and/or reduce or eliminate aggregation.

Heavy-chain only antibodies lack both light chains and a functional CH1 domain and thus rely exclusively on a heavy chain variable domain for antigen binding. Heavy-chain only antibodies are produced naturally in the Camelidae family (e.g., camels, dromedaries, llamas, vicunas, guanaco, and alpacas) as well as in cartilaginous fish (e.g., sharks). In addition to natural sources, transgenic mammals (e.g., mice) have been engineered to express heavy-chain only antibodies. Such transgenic mammals include, for example, transgenic animals described in U.S. Patent Publications 2015/0289489 A1, 2023/0270086 A1, and 2023/0062964 A1, and 2020/0267951 A1, each of which is incorporated herein by reference.

In some embodiments, an sdAb is generated by immunizing an animal that produces heavy-chain only antibodies, including a natural producer (e.g., camelids, sharks) or an engineered non-human mammal (e.g., a transgenic mouse), to obtain heavy-chain only antibodies. Such antibodies may be screened to identify those having desirable properties (e.g., target affinity). Once produced and identified, the variable region of the antibody heavy chain is cloned to construct a single domain antibody consisting of only one heavy chain variable region.

sdAbs can also be obtained by immunizing animals that generate traditional antibodies (e.g., rabbits) followed by screening for VHs having high binding affinity in the absence of their cognate light chain (see e.g., Shinozaki et al., 2017, Scientific Reports, 7(1):5794).

sdAbs can be humanized by replacing natural (e.g., camelid) framework sequences with human sequences (see, e.g., Vincke, 2009, The Journal of Biological Chemistry, 285(5):3273-3284; Murakami et al., 2022, Antibodies, 11(1):10; and U.S. Patent Publication No. 2016/0237142 A1, incorporated herein by reference).

Fully human sdAbs can also be obtained using human VH single domains (see, e.g., Rouet et al., 2015, The Journal of Biological Chemistry, 290(19):11905-11917).

Additional methods for producing heavy-chain only antibodies and/or sdAbs are recognized in the art and include, for example, those described in Muyldermans, 2021, The FEBS journal, 288(7):2084-2102.

In some cases, an sdAb is engineered to enhance certain properties. For example, in some embodiments, a disulfide bond is introduced within a VHH to increase stability (see e.g., Hagihara et al., 2007, The Journal of Biological Chemistry, 282(50):36489-36495).

### 6.4. Antigen Targeting Moieties

MBMs of the present disclosure comprise four antigen targeting moieties. In some embodiments, the antigen targeting moiety is a T cell antigen (TCA) targeting moiety, e.g., as described in Section 6.4.1. In some embodiments, the antigen targeting moiety tumor antigen targeting moiety, e.g., as described in Section 6.4.1.1.2. In some embodiments, the antigen targeting moiety is a low-density antigen targeting moiety, e.g., as described in Section 6.4.3. In some embodiments, the antigen targeting moiety is an HLA-bound peptide antigen targeting moiety, e.g., as described in Section 6.4.4.

As will be recognized by the skilled artisan, an antigen targeting moiety of an MBM of the disclosure may be considered as being of multiple types based on the antigen to which the antigen targeting moiety specifically binds. For example, an antigen targeting moiety may be both a tumor antigen targeting moiety and an HLA-bound peptide antigen targeting moiety where the HLA-bound peptide is a tumor-specific peptide. Similarly, an antigen targeting moiety may be both a low-density antigen targeting moiety and an HLA-bound peptide antigen targeting moiety, for example, where the HLA-bound peptide is present at no more than 5000 copies on the surface of a target cell.

Exemplary antigen targeting moieties are described in further detail below.

### 6.4.1. T Cell Antigen (TCA) Targeting Moieties

In some embodiments, an MBM of the present disclosure comprises one or more (e.g., two) T cell antigen (TCA) targeting moieties. A TCA targeting moiety generally binds to a specific T cell antigen. In some embodiments, both of the TCA targeting moieties specifically bind to the same T cell antigen (e.g., both TCA targeting moieties specifically bind to CD3). In such cases, each TCA targeting moiety may bind to the same epitope (e.g., the same epitope on CD3) or different epitopes (e.g., different epitopes of CD3). Where the TCA targeting moieties each bind to the same epitope, they may have identical antigen-binding domains (ABDs), or different ABDs. In some embodiments, the TCA targeting moieties each bind to a different T cell antigen (e.g., one TCA targeting moiety binds to one component of the TCR while the other TCA targeting moiety binds to a different component of the TCR). Exemplary target molecules recognized by the TCA targeting moieties of the disclosure, and sequences of ABDs of such targeting moieties, are described in Section 6.4.1.1.

The TCA targeting moieties of the disclosure include targeting moieties (e.g., antibodies and antigen-binding fragments thereof) that bind the TCA (e.g., CD3) with high affinity. The TCA targeting moieties of the disclosure also include targeting moieties (e.g., antibodies and antigen-binding fragments thereof) that bind the TCA (e.g., CD3) with moderate or low affinity, depending on the therapeutic context and particular targeting properties that are desired. In some embodiments, “low affinity” (also “weak affinity”) describes TCA targeting moieties that bind the TCA with a K_{D} or EC₅₀ (e.g., as measured in a surface plasmon resonance assay) of greater than 10⁻⁶ M, greater than 10⁻⁷ M, greater than 10⁻⁸ M, or greater than 10⁻⁹ M.

The TCA targeting moiety is generally an antigen binding moiety, for example an antibody or an antigen-binding portion of an antibody, e.g., an scFv, as described in Section 6.3.1, or a Fab, as described in Section 6.3.2.

### 6.4.1.1. T Cell Antigens

In general, a target for a TCA targeting moiety is any molecule present on or expressed by a T cell. In certain embodiments, a TCA targeting moiety of the disclosure targets a cell surface molecule of a T cell. Example targets for a TCA targeting moiety of the disclosure include, but are not limited to, CD3, the T cell receptor (e.g., TCRap or TCRyb), CD27, CD28, 4-1BB (CD137), OX40, CD30, CD40, ICOS, lymphocyte function-associated antigen-1 (LFA-1), CD2, CD7, LIGHT, NKG2C, and B7-H3. In some embodiments, the target for the TCA targeting moiety is CD3. In some embodiments, the target for the TCA targeting moiety is the T cell receptor (e.g., TCRαβ or TCRγδ). The epitope of the TCA targeting moiety can be an individual polypeptide (e.g., CD3 epsilon) or a multimeric component of a protein complex (e.g., the TCRαβ dimer or the TCRγδ dimer of the T cell receptor complex).

### 6.4.1.1.1. CD3 and TCR Targeting Moieties

In particular embodiments, a TCA targeting moiety of the present disclosure is a CD3 targeting moiety and/or a TCR targeting moiety. A CD3 targeting moiety may be or comprise an antigen-binding domain from an anti-CD3 antibody. A TCR targeting moiety may be or comprise an antigen-binding domain from an anti-TCR antibody.

Exemplary anti-CD3 and anti-TCR antibodies or antibody sequences are set forth in Table T-1 below, upon which the TCA targeting moiety can be based.

TABLE T-1
Exemplary Anti-CD3 and Anti-TCR Antibodies

| Target | Antibody Name and/or Binding Sequences |
|---|---|
| CD3 | The CD3-binding portion of Catumaxomab |
| CD3 | The CD3-binding portion of ertumaxomab |
| CD3 | The CD3-binding portion of anti-PSMA/anti-CD3 antibodies described in |
| | WO2011121110A1 |
| CD3 | Anti-CD3 antibody sequences in US10266593B2 |
| CD3 | Anti-CD3 antibody sequences in US 8846042B2 |
| CD3 | Anti-CD3 antibody sequences US 2016/0355600 |
| CD3 | Anti-CD3 antibody sequences in WO 2014/110601 |
| CD3 | Anti-CD3 antibody sequences in WO 2014/145806 |
| CD3 | Anti-CD3 antibody sequences in US 10,066,015 |
| CD3 | Anti-CD3 antibody sequences in WO 2019/034580 |
| CD3 | Anti-CD3 antibody sequences in WO 2014/056783 |
| CD3 | Anti-CD3 antibody sequences in WO 2013/055809 A1 |
| CD3 | Anti-CD3 antibody sequences in US 10,066,016 |
| CD3 | Anti-CD3 antibody sequences in US 2010/0150918 |
| CD3 | The CD3-binding portion of MT110 |
| CD3 | The CD3-binding portion of Acapatamab (AMG160) |
| CD3 | The CD3-binding portion of AMG199 |
| CD3 | The CD3-binding portion of AMG330 |
| CD3 | The CD3-binding portion of AMG427 (Emirodatamab) |
| CD3 | The CD3-binding portion of AMG562 |
| CD3 | The CD3-binding portion of AMG596 |
| CD3 | The CD3-binding portion of AMG673 |
| CD3 | The CD3-binding portion of AMG701 (Pavurutamab) |
| CD3 | The CD3-binding portion of Tarlatamab (AMG757) |
| CD3 | The CD3-binding portion of AMG910 (Gresonitamab) |
| CD3 | The CD3-binding portion of BAY2010112 (Pasotuxizumab) |
| CD3 | The CD3-binding portion of AMG420 |
| CD3 | The CD3-binding portion of AMG424 |
| CD3 | The CD3-binding portion of AMG509 |
| CD3 | The CD3-binding portion of AMV564 |
| CD3 | The CD3-binding portion of APVO436 |
| CD3 | The CD3-binding portion of Alnuctamab (CC-93269; BMS-986349) |
| CD3 | The CD3-binding portion of ERY974 |
| CD3 | The CD3-binding portion of A-319 |
| CD3 | The CD3-binding portion of GEM333 |
| CD3 | The CD3-binding portion of GEM3PSCA |
| CD3 | The CD3-binding portion of Cevostamab |
| CD3 | The CD3-binding portion of Runimotamab |
| CD3 | The CD3-binding portion of GEN1044 |
| CD3 | Epcoritamab (GEN3013) |
| CD3 | The CD3-binding portion of HPN424 |
| CD3 | The CD3-binding portion of ISB1302 |
| CD3 | The CD3-binding portion of ISB1342 |
| CD3 | The CD3-binding portion of IGM-2323 |
| CD3 | The CD3-binding portion of IMC-F106C |
| CD3 | The CD3-binding portion of IMC-C103C |
| CD3 | The CD3-binding portion of IMCnyeso |
| CD3 | The CD3-binding portion of JNJ-63709178 |
| CD3 | The CD3-binding portion of JNJ-63898081 (JNJ-081) |
| CD3 | The CD3-binding portion of Teclistamab |
| CD3 | The CD3-binding portion of Talquetamab (JNJ-64407564) |
| CD3 | The CD3-binding portion of JNJ-67571244 |
| CD3 | The CD3-binding portion of MGD007 |
| CD3 | The CD3-binding portion of Orlotamab (MGD009) |
| CD3 | The CD3-binding portion of Duvortuxizumab |
| CD3 | The CD3-binding portion of Flotetuzumab (MGD006) |
| CD3 | The CD3-binding portion of MCLA-117 |
| CD3 | The CD3-binding portion of PF-06671008 |
| CD3 | The CD3-binding portion of Elranatamab |
| CD3 | The CD3-binding portion of Odronextamab |
| CD3 | The CD3-binding portion of REGN5458 |
| CD3 | The CD3-binding portion of REGN5459 |
| CD3 | The CD3-binding portion of REGN4018 |
| CD3 | The CD3-binding portion of Glofitamab (RO7082859) |
| CD3 | The CD3-binding portion of RO6958688 (RG7802) |
| CD3 | The CD3-binding portion of SAR440234 |
| CD3 | The CD3-binding portion of TNB-383B |
| CD3 | The CD3-binding portion of M802 |
| CD3 | The CD3-binding portion of Xmab 13676 |
| CD3 | The CD3-binding portion of Xmab 18087 |
| CD3 | The CD3-binding portion of Vibecotamab (XmAb14045) |
| CD3 | The CD3-binding portion of Nivatrotamab (Hu3F8-BsAb) |
| CD3 | Anti-CD3 antibody sequences in US20190211100 |
| CD3 | Anti-CD3 antibody sequences in EP1629011B |
| CD3 | VH of SEQ ID NOS. 90 and 98 disclosed in US 2021/0206865 A1 |
| | CDR-H1 of SEQ ID NO: 92 and 100 disclosed in US 2021/0206865 A1 |
| | CDR-H2 of SEQ ID NO: 94 and 102 disclosed in US 2021/0206865 A1 |
| | CDR-H3 of SEQ ID NO: 96 and 104 disclosed in US 2021/0206865 A1 |
| | HC of SEQ ID NO: 127 or SEQ ID NO: 128 disclosed in US 2021/0206865 A1 |
| | LC of SEQ ID NO: 129 or SEQ ID NO: 132 disclosed in US 2021/0206865 A1 |
| CD3 | Anti-CD3 Heavy chain of SEQ ID NO: 2 disclosed in US 2021/0206870 A1 |
| | Anti-CD3 VH SEQ ID NO: 5 disclosed in US 2021/0206870 A1 |
| | Anti-CD3 VL of SEQ ID NO: 6 disclosed in US 2021/0206870 A1 |
| | Anti-CD3 CDR-H1 of SEQ ID NO: 10 disclosed in US 2021/0206870 A1 |
| | Anti-CD3 CDR-H2 of SEQ ID NO: 11 disclosed in US 2021/0206870 A1 |
| | Anti-CD3 CDR-H3 of SEQ ID NO: 12 disclosed in US 2021/0206870 A1 |
| CD3 | Anti-CD3 VH of SEQ ID NO: 92, 102, 112, 122, 132, 142, 156, 166, 176, 186, |
| | 196 or 206 disclosed in US 2022/0119525 A1 |
| | Anti-CD3 CDR-H1 of SEQ ID NO: 93, 103, 113, 123, 133, 143, 157, 167, 177, |
| | 187, 197 or 207 disclosed in US 2022/0119525 A1 |
| | Anti-CD3 CDR-H2 of SEQ ID NO: 94, 104, 114, 124, 134, 144, 158, 168, 178, |
| | 188, 198 or 208 disclosed in US 2022/0119525 A1 |
| | Anti-CD3 CDR-H3 of SEQ ID NO: 95, 105, 115, 125, 135, 145, 159, 169, 179, |
| | 189, 199 or 209 disclosed in US 2022/0119525 A1 |
| | Anti-CD3 VL of SEQ ID NO: 96, 106, 116, 126, 136, 146, 152, 162, 172, 182, |
| | 192 or 202 disclosed in US 2022/0119525 A1 |
| | Anti-CD3 CDR-L1 of SEQ ID NO: 97, 107, 117, 127, 137, 147, 153, 163, 173, |
| | 183, 193 or 203 disclosed in US 2022/0119525 A1 |
| | Anti-CD3 CDR-L2 of SEQ ID NO. 98, 108, 118, 128, 138, 148, 154, 164, 174, |
| | 184, 194 or 204 disclosed in US 2022/0119525 A1 |
| | Anti-CD3 CDR-L3 of SEQ ID NO. 99, 109, 119, 129, 139, 149, 155, 165, 175, |
| | 185, 195 or 205 disclosed in US 2022/0119525 A1 |
| CD3 | L2K |
| CD3 | A2J |
| CD3 | 6G12 |
| CD3 | 1A4 |
| CD3 | OKT3 (Ortho Kung T3; Muromonab-CD3) |
| CD3 | Teplizumab (PRV-031; MGA03) |
| CD3 | Otelixizumab (TRX4) |
| CD3 | Anti-CD3 VH of SEQ ID NO: 2, 18, 34, 50, 66, 82, 98, 114, 130, 146, 162, |
| | 178, 194, 210, 226, 242, 258, 274, 290, 306, 322, 338, 354, 370, 386, 402, |
| | 418, 434, 450, 466, 482, 498, 514, 530, 546, 562, 578, 594, 610, 626, 642, |
| | 658, 674, 690, 706, 722, 738, 754, 770, 786, 802, 818, 834, 850, 866, 882, |
| | 898, 914, 930, 946, 962, 978, 994, 1010, 1026, 1042, 1050, 1058, 1066, |
| | 1074, 1082, 1090, 1098, 1106, 1114, 1122, 1130, 1138, 1146, 1154, 1162, |
| | 1170, 1178, 1186, 1194, 1202, 1210, 1218, or 1226 disclosed in US 9657102 |
| | B2 |
| | Anti-CD3 CDR-H1 of SEQ ID NO: 4, 20, 36, 52, 68, 84, 100, 116, 132, 148, |
| | 164, 180, 196, 212, 228, 244, 260, 276, 292, 308, 324, 340, 356, 372, 388, |
| | 404, 420, 436, 452, 468, 484, 500, 516, 532, 548, 564, 580, 596, 612, 628, |
| | 644, 660, 676, 692, 708, 724, 740, 756, 772, 788, 804, 820, 836, 852, 868, |
| | 884, 900, 916, 932, 948, 964, 980, 996, 1012, 1028, 1044, 1052, 1060, 1068, |
| | 1076, 1084, 1092, 1100, 1108, 1116, 1124, 1132, 1140, 1148, 1156, 1164, |
| | 1172, 1180, 1188, 1196, 1204, 1212, or 1220, 1228 disclosed in US 9657102 |
| | B2 |
| | Anti-CD3 CDR-H2 of SEQ ID NO: 6, 22, 38, 54, 70, 86, 102, 118, 134, 150, |
| | 166, 182, 198, 214, 230, 246, 262, 278, 294, 310, 326, 342, 358, 374, 390, |
| | 406, 422, 438, 454, 470, 486, 502, 518, 534, 550, 566, 582, 598, 614, 630, |
| | 646, 662, 678, 694, 710, 726, 742, 758, 774, 790, 806, 822, 838, 854, 870, |
| | 886, 902, 918, 934, 950, 966, 982, 998, 1014, 1030, 1046, 1054, 1062, 1070, |
| | 1078, 1086, 1094, 1102, 1110, 1118, 1126, 1134, 1142, 1150, 1158, 1166, |
| | 1174, 1182, 1190, 1198, 1206, 1214, or 1222, 1230 disclosed in US 9657102 |
| | B2 |
| | Anti-CD3 CDR-H3 of SEQ ID NO: 8, 24, 40, 56, 72, 88, 104, 120, 136, 152, |
| | 168, 184, 200, 216, 232, 248, 264, 280, 296, 312, 328, 344, 360, 376, 392, |
| | 408, 424, 440, 456, 472, 488, 504, 520, 536, 552, 568, 584, 600, 616, 632, |
| | 648, 664, 680, 696, 712, 728, 744, 460, 776, 792, 808, 824, 840, 856, 872, |
| | 888, 904, 920, 936, 952, 968, 984, 1000, 1016, 1032, 1048, 1056, 1064, |
| | 1072, 1080, 1088, 1096, 1104, 1112, 1120, 1128, 1136, 1144, 1152, 1160, |
| | 1168, 1176, 1184, 1192, 1200, 1208, 1216, or 1224, 1232 disclosed in US |
| | 9657102 B2 |
| | Anti-CD3 VL of SEQ ID NO: 10, 26, 42, 58, 74, 90, 106, 122, 138, 154, 170, |
| | 186, 202, 218, 234, 250, 266, 282, 298, 314, 330, 346, 362, 378, 394, 410, |
| | 426, 442, 458, 474, 490, 506, 522, 538, 554, 570, 586, 602, 618, 634, 650, |
| | 666, 682, 698, 714, 730, 746, 762, 778, 794, 810, 826, 842, 858, 874, 890, |
| | 906, 922, 938, 954, 970, 986, 1002, 1018, 1034, 1234, 1234, 1234, 1234, |
| | 1234, 1234, 1234, 1234, 1234, 1234, 1234, 1234, 1234, 1234, 1234, 1234, |
| | 1234, 1234, 1234, 1234, 1234, 1234, or 1234, 1234 disclosed in US 9657102 |
| | B2 |
| | Anti-CD3 CDR-L1 of SEQ ID NO: 12, 28, 44, 60, 76, 92, 108, 124, 140, 156, |
| | 172, 188, 204, 220, 236, 252, 268, 284, 300, 316, 332, 348, 364, 380, 396, |
| | 412, 428, 444, 460, 476, 492, 508, 524, 540, 556, 572, 588, 604, 620, 636, |
| | 652, 668, 684, 700, 716, 732, 748, 764, 780, 796, 812, 828, 844, 860, 876, |
| | 892, 908, 924, 940, 956, 972, 988, 1004, 1020, 1036, 1236, 1236, 1236, |
| | 1236, 1236, 1236, 1236, 1236, 1236, 1236, 1236, 1236, 1236, 1236, 1236, |
| | 1236, 1236, 1236, 1236, 1236, 1236, 1236, or 1236, 1236 disclosed in US |
| | 9657102 B2 |
| | Anti-CD3 CDR-L2 of SEQ ID NO. 14, 30, 46, 62, 78, 94, 110, 126, 142, 158, |
| | 174, 190, 206, 222, 238, 254, 270, 286, 302, 318, 334, 350, 366, 382, 398, |
| | 414, 430, 446, 462, 478, 494, 510, 526, 542, 558, 574, 590, 606, 622, 638, |
| | 654, 670, 686, 702, 718, 734, 750, 766, 782, 798, 814, 830, 846, 862, 878, |
| | 894, 910, 926, 942, 958, 974, 990, 1006, 1022, 1038, 1238, 1238, 1238, |
| | 1238, 1238, 1238, 1238, 1238, 1238, 1238, 1238, 1238, 1238, 1238, 1238, |
| | 1238, 1238, 1238, 1238, 1238, 1238, 1238, or 1238, 1238 disclosed in US |
| | 9657102 B2 |
| | Anti-CD3 CDR-L3 of SEQ ID NO. 16, 32, 48, 64, 80, 96, 112, 128, 144, 160, |
| | 176, 192, 208, 224, 240, 256, 272, 288, 304, 320, 336, 352, 368, 384, 400, |
| | 416, 432, 448, 464, 480, 496, 512, 528, 544, 560, 576, 592, 608, 624, 640, |
| | 656, 672, 688, 704, 720, 736, 752, 768, 784, 800, 816, 832, 848, 864, 880, |
| | 896, 912, 928, 944, 960, 976, 992, 1008, 1024, 1040, 1240, 1240, 1240, |
| | 1240, 1240, 1240, 1240, 1240, 1240, 1240, 1240, 1240, 1240, 1240, 1240, |
| | 1240, 1240, 1240, 1240, 1240, 1240, 1240, or 1240, 1240 disclosed in US |
| | 9657102 B2 |
| | (see also US 9657102 B2 at Table 1, incorporated herein by reference) |
| CD3 | VH of SEQ ID NOs: 2, 10, 18, 26, 34, 42, 50, 58, 66, 74, 82, 90, 98, 106, 114, |
| | 122, 130, 138, 146, and 154 disclosed in US 2023/0068129 A1 |
| | VL of SEQ ID NO: 162 disclosed in US 2023/0068129 A1 |
| | (see also Tables 1-4 of US 2023/0068129 A1, incorporated herein by |
| | reference) |
| CD3 | VH: |
| | EVQLVESGGGLVQPGRSLRLSCAASGFTFDDYSMHWVRQAPGKGLEWVS |
| | GISWNSGSKGYADSVKGRFTISRDNAKNSLYLQMNSLRAEDTALYYCAKYG |
| | SGYGKFYHYGLDVWGQGTTVTVSS (SEQ ID NO: 2) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 3) |
| CD3 | VH: |
| | EVQLVESGGGLVQPGRSLRLSCAASGFTFDDYSMHWVRQAPGKGLEWVS |
| | GISWNSGSIGYADSVKGRFTISRDNAKNSLYLQMNSLRAEDTALYYCAKDG |
| | SGYGKFYYYGMDVWGQGTTVTVSS (SEQ ID NO: 4) |
| | VL |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 3) |
| CD3 | VH: |
| | EVQLVESGGGLVQPGRSLRLSCAASGFTFDDYSMHWVRQAPGKGLEWVS |
| | GISWNSGSIGYADSVKGRFTISRDNAKNSLYLQMNSLRAEDTALYYCAKYG |
| | SGYGKFYYYGMDVWGQGTTVTVSS (SEQ ID NO: 5) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 3) |
| CD3 | VH: |
| | EVQLVESGGGLVQPGRSLRLSCAASGFTFDDYSMHWVRQAPGKGLEWVS |
| | GISWNSGSKGYADSVKGRFTISRDNAKNSLYLQMNSLRAEDTALYYCAKYG |
| | SGYGKFYHYGLDVWGQGTTVTVSS (SEQ ID NO: 2) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 3) |
| TCRαβ | BMA031 sequences disclosed in US 2012/0034221 |
| TCRγδ | 6TCS1 antibody disclosed in US 5,980,892 |

In some aspects, the TCA targeting moiety competes with an antibody set forth in Table T-1 for binding to the target (e.g., CD3 or a T cell receptor). In further aspects, the TCA targeting moiety comprises CDRs having CDR sequences of an antibody set forth in Table T-1. In some embodiments, the TCA targeting moiety comprises all 6 CDR sequences of an antibody set forth in Table T-1. In other embodiments, the TCA targeting moiety comprises at least the heavy chain CDR sequences (CDR-H1, CDR-H2, CDR-H3) or an antibody set forth in Table T-1 and the light chain CDR sequences of a universal light chain. In further aspects, a TCA targeting moiety comprises a VH comprising the amino acid sequence of the VH of an antibody set forth in Table T-1. In some embodiments, the TCA targeting moiety further comprises a VL comprising the amino acid sequence of the VL of an antibody set forth in Table T-1. In other embodiments, the TCA targeting moiety further comprises a universal light chain VL sequence.

The CD3 targeting moieties of the disclosure include targeting moieties (e.g., antibodies and antigen-binding fragments thereof) that bind CD3 (e.g., human CD3) with high affinity. The CD3 targeting moieties of the disclosure also include targeting moieties (e.g., antibodies and antigen-binding fragments thereof) that bind the CD3 (e.g., human 003) with moderate or low affinity, depending on the therapeutic context and particular targeting properties that are desired. Accordingly, in some embodiments, a CD3 targeting moiety of the present disclosure has a KD as measured in a surface plasmon resonance assay (e.g., at 25° C.) of greater than 10⁻⁶ M, greater than 10⁻⁷ M, greater than 10⁻⁸ M, or greater than 10⁻⁹ M, including any range or value derivable therein (e.g., between 10⁻⁶ M and 10⁻⁷ M, between 10⁻⁶ M and 10⁻⁸ M, between 10⁻⁶ M and 10⁻⁹ M, between 10⁻⁷ M and 10⁻⁸ M, or between 10⁻⁷ M and 10⁻⁹ M, or between 10⁻⁸ M and 10⁻⁹ M).

### 6.4.1.1.2. CD28

In particular embodiments, a TCA targeting moiety of the present disclosure is a CD28 targeting moiety and. A CD28 targeting moiety may be or comprise an antigen-binding domain from an anti-CD28 antibody.

Exemplary anti-CD28 antibodies or antibody sequences are set forth in Table T-2 below, upon which the TCA targeting moiety can be based.

TABLE T-2
Exemplary Anti-CD28 Antibodies

| Target | Antibody Name and/or Binding Sequences |
|---|---|
| CD28 | Theralizumab (TGN1412) |
| CD28 | FK734 |
| CD28 | CD28A-49 |
| CD28 | Anti-CD28 antibody sequences in WO 2020/127618 A1 |
| CD28 | Anti-CD28 antibodies and CD28 portion of anti-PSMA/anti-CD28 antibodies |
| | described in WO 2019/246514 A1 |
| CD28 | The CD28 portion of anti-CD3/anti-CD28 antibodies described in WO |
| | 2021/260064 A1 |
| CD28 | The CD28 portion of anti-CD3/anti-CD28 antibodies described in WO |
| | 2009/062001 A1 |
| CD28 | The CD28 portion of anti-EGFR/anti-CD28 antibody described in WO |
| | 2020/198009 A1 |
| CD28 | The CD28 portion of anti-CD3/anti-CD28/anti-CD137 antibodies described in |
| | WO 2021/173307 A1 |
| CD28 | The CD28 portion of anti-CD38/anti-CD28/anti-CD3 antibodies described in WO |
| | 2019/074973 A2 |
| CD28 | The CD28 portion of anti-CEA/anti-CD3/anti-CD28 antibodies described in WO |
| | 2005/095456 A1 |
| CD28 | The CD28 portion of anti-MUC16/anti-CD28 antibodies described in WO |
| | 2020/132024 A1 |
| CD28 | The CD28 portion of anti-CD28/anti-CD22 antibodies described in WO |
| | 2020/132066 A1 |
| CD28 | Anti-CD28 antibody sequences in WO 2020/127628 A1 |
| CD28 | Anti-CD28 antibody sequences in WO 2022/061098 A1 |
| CD28 | Anti-CD28 antibody sequences in WO 2003/042231 A2 |
| CD28 | Anti-CD28 antibody sequences in WO 2014/209868 A1 |
| CD28 | Anti-CD28 antibody sequences in WO 2022/096536 A1 |
| CD28 | VH: |
| | EVQLVESGGGLVQPGGSLRLSCAASGFTFSRNNMHWVRQAPGKGLEYVSGIS |
| | SNGGRTYYADSVKGRFTISRDNSKNTLYLQMGGLRAADMAVYFCTRDDELLSF |
| | DYWGQGTLVTVSS (SEQ ID NO: 6) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPPITFGQGTRLEIK |
| | (SEQ ID NO: 207) |

In some aspects, the TCA targeting moiety competes with an antibody set forth in Table T-2 for binding to the target. In further aspects, the TCA targeting moiety comprises CDRs having CDR sequences of an antibody set forth in Table T-2. In some embodiments, the TCA targeting moiety comprises all 6 CDR sequences of an antibody set forth in Table T-2. In other embodiments, the TCA targeting moiety comprises at least the heavy chain CDR sequences (CDR-H1, CDR-H2, CDR-H3) or an antibody set forth in Table T-2 and the light chain CDR sequences of a universal light chain. In further aspects, a TCA targeting moiety comprises a VH comprising the amino acid sequence of the VH of an antibody set forth in Table T-2. In some embodiments, the TCA targeting moiety further comprises a VL comprising the amino acid sequence of the VL of an antibody set forth in Table T-2. In other embodiments, the TCA targeting moiety further comprises a universal light chain VL sequence.

### 6.4.2. Tumor Antigen Targeting Moieties

In some embodiments, an MBM of the present disclosure comprises at least one targeting moiety that binds specifically to a target molecule expressed by a tumor cell or in the tumor cell environment, e.g., an extracellular matrix (“ECM”) antigen, a tumor reactive lymphocyte antigen, a cell surface molecule of tumor or viral lymphocytes, a checkpoint inhibitor, or a tumor-associated antigen (TAA), collectively referred to herein as a “tumor antigen targeting moieties.” The skilled artisan would recognize that the foregoing categories of target molecules are not mutually exclusive and thus a given target molecule may fall into more than one of the foregoing categories of target molecules. For example, some molecules may be considered both TAAs and ECM proteins. Preferably, the ECM antigen, tumor reactive lymphocyte antigen, cell surface molecule of tumor or viral lymphocytes, checkpoint inhibitor, or TAA is a human antigen. The antigen may or may not be present on normal cells. Certain aspects are directed to MBMs comprising at least one targeting moiety that binds specifically to a TAA.

It is anticipated that any type of tumor and any type of ECM antigen, tumor reactive lymphocyte antigen, cell surface molecule of tumor or viral lymphocytes, checkpoint inhibitor, or TAA may be targeted by an antigen targeting moiety of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Non-limiting examples of ECM antigens include syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In other embodiments, the target molecule is LAG3. In some embodiments, where the target molecule is a checkpoint inhibitor, the tumor antigen targeting moiety is non-blocking or poorly-blocking of ligand-receptor binding. Examples of non-blocking or poorly-blocking anti-PD1 antibodies includes antibodies having VH/VL amino acid sequences of SEQ ID Nos: 2/10 of POT Pub. No. WO2015/112800A1; SEQ ID Nos: 16/17 of U.S. Pat. No. 11,034,765 B2; SEQ ID Nos. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID Nos 23/24, 3/4 and 11/12 of US Pub. US2022/0056126A1.

Exemplary tumor antigens are set forth in Table T-3 below, together with references to exemplary antibodies or antibody sequences upon which the tumor antigen targeting moiety can be based.

TABLE T-3
Exemplary Target Molecules

| Target (Tumor Antigen) | Antibody Name and/or Binding Sequences |
|---|---|
| 1-92-LFA-3 | Amevive ™ (alefacept) |
| 5T4 | GEN1044 |
| Activin Receptor Type II | Bimagrumab |
| | VH: SEQ ID Nos: 107, 109 of U.S. Pat. No. 8,388,968 B2 |
| | VL: SEQ ID Nos: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID NO: 21, 26 |
| | or 31 of US 2021/0171641 A1. |
| | VL: the VL sequence of the light chain of SEQ ID NO: 20, 22 or |
| | 30 of US 2021/0171641 A1. |
| B7-H3 (CD276) | VH: the VH sequence of the heavy chain of SEQ ID NO: 21, 29 |
| | or 37 of US 2019/0002563 A1. |
| | VL: the VL sequence of the light chain of SEQ ID NO: 17, 25 or |
| | 33 of US 2019/0002563 A1. |
| B7-H3 (CD276) | VH: the VH sequence of the heavy chain of SEQ ID NO: 146, |
| | 147 or 148 of U.S. Pat. No. 10,640,563. |
| | VL: the VL sequence of the light chain of SEQ ID NO: 143, 144 |
| | or 145 of U.S. Pat. No. 10,640,563. |
| BAFF/B Lymphocyte | Benlysta ™ (velimumab) |
| Stimulator | |
| BAFF/B Lymphocyte | VH: amino acids 1-123 of SEQ ID NO: 327 of |
| Stimulator | U.S. Pat. No. 7,138,501 |
| | VL: amino acids 139-249 of SEQ ID NO: 327 of |
| | U.S. Pat. No. 7,138,501. |
| BAFF/B Lymphocyte | VH: amino acids 1-126 of SEQ ID NO: 1321 of |
| Stimulator | U.S. Pat. No. 7,605,236; |
| | VL: amino acids 143-251 of SEQ ID NO: 1049 of |
| | U.S. Pat. No. 7,605,236. |
| BAFF/B Lymphocyte | Belimumab |
| Stimulator | |
| BCMA | VH: the VH sequence of the heavy chain of SEQ ID NO. 126 |
| | of US 2021/0206865 A1 |
| | VL: the VL sequence of the light chain of SEQ ID NO. 129 or |
| | SEQ ID NO. 132 of US 2021/0206865 A1 |
| CA125 | Igobumab |
| CA125 | OvaRex ™ (oregobumab) |
| Cadherin | The antibodies described in US Pub. No. US 2006/0039915. |
| N-cadherin | An antibody that binds to the amino acid sequence of SEQ ID |
| | NO: 10, 17 or 18 of US Pub. No. US 2010/0278821. |
| CD11a | Raptiva ™ (efalizumab) |
| | Sequence in Werther et al., 1996, The Journal of Immunology |
| | 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID NO: 124 of |
| | US Patent Pub. US 2017/0002060 A1 |
| | VL: the VL sequence of the light chain of SEQ ID NO: 125 of |
| | US Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular immunology |
| | 30: 1361-1367 |
| CD33 | Lintuzumab |
| CD38 | Darzalex ™ (daratumumab) |
| CD38 | IB4, HB7 CS/2, clone 90 and NIM-R5 as disclosed in PCT |
| | Pub. WO2015/009726A2 and references cited therein. |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US 2017/0002060 A1 |
| | VL: SEQ ID NO: 2 of US Patent Pub. US 2017/0002060 A1 |
| CD70 | Blenrep ™ (borsetuzumab mafodotin) |
| CD123 | Flotetuzumab |
| CD206 | Anti-CD206 antibodies having a VH a of SEQ ID NO. 2 and a |
| | VL of SEQ ID NO: 4 of WO2003/040169A2 |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID Nos: 4-6 |
| | of U.S. Pat. No. 8,771,690 B2 |
| | CDR-L1, CDR-L2 and CDR-L3 sequences of SEQ ID Nos: 1-3 |
| | of U.S. Pat. No. 8,771,690 B2 |
| CEA | CEA binding portion of RO6958688/RG7802 from clinical trial |
| | NCT02324257 |
| CEA | Cibisatamab |
| CEA | CEA binding portion of MEDI-565/MT110/AMG211 from |
| | clinical trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Clec9a | Anti-Clec9a antibodies having VH and VL amino acids of SEQ |
| | ID Nos. 43 and 48 of PCT Pub. No. WO2009/026660A1 |
| Clec9a | Anti-Clec9a antibodies having a VH a of SEQ ID NO. 38 and a |
| | VL of SEQ ID NO: 37 or SEQ ID NO: 43 of PCT Pub. No. |
| | WO2022/073062A1 |
| | Anti-Clec9a antibodies having a VH a of SEQ ID NO. 8 and a |
| | VL of SEQ ID NO: 7 of PCT Pub. No. WO2022/073062A1 |
| Collagen alpha-4 chain | TRC093 (MT293) |
| Collagen | The collagen binding antibody fragment described in Liang et |
| | al., 2016, Sci. Rep. 5, 18205; doi: 10.1038/srep18205 (2016). |
| Collagen Type I | Cetuximab (Erbitux) |
| Collagen type X | The amino acid sequences of SEQ ID NO: 1 or 2 of PCT Pub |
| | No. WO 2019/020797. |
| Collagen type X | The amino acid sequences of SEQ ID NO: 1 of PCT Pub No. |
| | WO 2014/180992. |
| Collagen type X | Antibody X34 as described in I. Girkontaite et al., |
| | “Immunolocalization of type X collagen in normal fetal and |
| | adult osteoarthritic cartilage with monoclonal |
| | antibodies,” Matrix Biol 15, 231-238 (1996). |
| Collagen type X | Antibodies X53 or 1H8 or ARC0659 or JF0961 collagen X |
| | polyclonal antibody sold under catalog number PA5-115039 or |
| | PA5-116871 or PA5-97603 or PA5-49198 from ThermoFisher |
| | Scientific. |
| Collagen type X | Antibody sold under catalog number RDI-COLL10abr from |
| | RDI. |
| Complement C5 | Soliris ™ (eculizumab) |
| | VH: amino acids 1-122 of SEQ ID NO: 10 of |
| | U.S. Pat. No. 6,355,245 |
| | VL: amino acids 3-110 of SEQ ID NO: 9 of |
| | U.S. Pat. No. 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DEC-205 | Anti-DEC-205 antibodies having a VH/VL pair of SEQ ID |
| | NOS. 4/10, 16/22, 28/34, 40/46, 52/58, and 76/82 of PCT Pub. |
| | WO2009/061996A2 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | Mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID Nos: 80, 84, 88, 92 or 96 |
| | VL: SEQ ID Nos: 82, 86, 90, 94 or 98 |
| EpCAM | G8.8 |
| | HC: SEQ ID NO: 4 of US Patent Pub. No. US 2020/0317806 A1 |
| | HL: SEQ ID NO: 3 of US Patent Pub. No. US 2020/0317806 A1 |
| EpCAM | VH: SEQ ID Nos: 17-22 of WO 2021/211510 A2. |
| | VL: SEQ ID NO: 15-16 of WO 2021/211510 A2. |
| EpCAM | Removab ™ (catumaxomab) |
| EpCAM | Vicineum ™ (oportuzumab monatox) |
| EpCAM | M701 |
| F protein of RSV | Synagic ™ (palivizumab) |
| GD2 | 3F8 |
| Glycoprotein receptor | ReoPro ™ (abiciximab) |
| IIb/IIIa | |
| gpA33 | MGD007 |
| GPC3 | ERY974 |
| GUCY2C | PF-07062119 |
| Heparanase | An antibody selected from HP130, HP 239, HP 108.264, HP |
| | 115.140, HP 152.197, HP 110.662, HP 144.141, HP 108.371, |
| | HP 135.108, HP 151.316, HP 117.372, HP 37/33, HP3/17, HP |
| | 201 or HP 102 or an amino acid sequence of SEQ ID NO: 1-11 |
| | described in US Patent Pub. US 2004/0170631. |
| Her2 | Herceptin ™ (trastuzumab) |
| Her2 | Aldesleukin (proleukine) |
| Her2 | Sargramustim (Leucine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446,175. |
| | VL: SEQ ID NO: 2 of U.S. Pat. No. 7,446,175 |
| IL12/IFN3 | Stelara ™ (ustekinumab) |
| IL1Ra | Antril ™, Kineret ™ (ankinra) |
| IFNR | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| IL12/IFN3 p40 subunit | Stelara ™ (ustekinumab) |
| | VH: SEQ ID NO: 7 of U.S. Pat. No. 6,902,734 |
| | VL: SEQ ID NO: 8 of U.S. Pat. No. 6,902,734 |
| Integrin α4 | Tysabri ™ (natalizumab) |
| | VH: SEQ ID Nos: 11-13 of U.S. Pat. No. 5,840,299 |
| | VL: SEQ ID Nos: 7-8 of U.S. Pat. No. 5,840,299 |
| Integrin α4 β7 | Entyvio ™ (vedolizumab) |
| | HC: SEQ ID NO: 2 of US Patent Pub. US 2012/0282249. |
| | LC: SEQ ID NO: 4 of US Patent Pub. US 2012/0282249. |
| Integrin α5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US |
| | Patent Pub. US 2022/0089744. |
| | VL: SEQ ID NO: 4, 16, 18, 20, 22, 44-57 or 107-116 of US |
| | Patent Pub. US 2022/0089744. |
| KIR | Anti-KIR antibodies having a VH of SEQ ID NO: 5 and a VL of |
| | SEQ ID NO: 3 of PCT Pub. WO2014/066532A1 |
| KIR | Anti-KIR antibodies having a VH of SEQ ID NO: 1 and a VL of |
| | SEQ ID NO: 2 of PCT Pub. WO2012/160448A2 |
| | Anti-KIR antibodies having a VH of SEQ ID NO: 3 and a VL of |
| | SEQ ID NO: 4 of PCT Pub. WO2012/160448A2 |
| LAG3 | Relatlimab (BMS-98016) |
| LAG3 | Sym022 |
| LAG3 | HLX26 |
| LAG3 | TSR-033 |
| LAG3 | ABL501 |
| LAG3 | INCAGN02385 |
| LAG3 | Fianlimab (REGN3767) |
| LAG3 | RO7247669 |
| LAG3 | EMB-02 |
| LAG3 | FS118 |
| LAG3 | GSK2831781 |
| LAG3 | IBI323 |
| LAG3 | IBI110 |
| LAG3 | LAG525 |
| LAG3 | XmAb ®22841 |
| LAG3 | LBL-007 |
| LAG3 | VH: SEQ ID NO: 1, 8, 10 or 12 of U.S. Pat. No. 9,902,772. |
| | VL: SEQ ID NO: 2, 3, 4, 5, 6, 7, 9, 11, 13 or 14 of |
| | U.S. Pat. No. 9,902,772. |
| LAG3 | VH: SEQ ID NO: 182 of US Patent Pub. US 2021/0095026. |
| | VL: SEQ ID NO: 88 of US Patent Pub. US 2021/0095026. |
| LAG3 | Antibodies having VH/VL amino acid sequences of SEQ ID Nos |
| | 23/24, 3/4 and 11/12 of US Pub. US2022/0056126A1. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences having |
| | the amino acid sequences of any one of the following SEQ ID |
| | NO: pairs from US 2018/0118848A1: 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274,160. |
| | VL: SEQ ID NO: 2 of U.S. Pat. No. 11,274,160. |
| NGF | (tanezumab) |
| NKH1A | The monoclonal antibody deposited with ATCC and |
| | assigned accession no. HB8564, as described in |
| | U.S. Pat. No. 4,772,552A |
| NKP46 | Anti-NKP46 antibodies having CDR-H1, CDR-H2 and CDR- |
| | H3 sequences of SEQ ID Nos: 4, 6 and 8 and CDR-L1, CDR- |
| | L2 and CDR-L3 sequences of SEQ ID Nos: 12, 14 and 16 of |
| | PCT Pub. WO2018/047154A1 |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106/BMS-936558 (nivolumab), a human IgG4 mAb with |
| | the structure described in WHO Drug Information, Vol. 27, No. 1, |
| | pages 68-69 (2013) and whose heavy and light chain sequences |
| | are disclosed in FIG. 7 of US Pub. No. US20190270812A1 |
| | HC: SEQ ID NO: 23 of US Pub. No. US20190270812A1 |
| | LC: SEQ ID NO: 24 of US Pub. No. US20190270812A1 |
| PD1 | MK-3475 (pembrolizumab), a humanized IgG4 mAb with the |
| | structure described in WHO Drug Information, Vol. 27, No. 2, |
| | pages 161-162 (2013) and whose heavy and light chain |
| | sequences are disclosed in FIG. 6 of US Pub. No. |
| | US20190270812A1 |
| | HC: SEQ ID NO: 21 of US Pub. No. US20190270812A1 |
| | LC: SEQ ID NO: 22 of US Pub. No. US20190270812A1 |
| PD1 | REGN2810 (disclosed as H4H7798N in US Pub No. |
| | 20150203579) |
| | HC: SEQ ID NO: 330 of US Pub. No. 20150203579 |
| | LC: SEQ ID NO: 331 of US Pub. No. 20150203579 |
| PD1 | H2M7791N disclosed in US Pub No. 20150203579 as having |
| | the following heavy and light chain variable domains: |
| | VH: SEQ ID NO: 98 of US Pub. No. 20150203579 |
| | VL: SEQ ID NO: 106 of US Pub. No. 20150203579 |
| PD1 | Anti-PD1 antibodies having CDR H1-H3 and CDR L1-L3 |
| | sequences corresponding to the following SEQ ID Nos. of |
| | U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; and |
| | h) SEQ ID Nos: 60, 61, 62, 63, 64, and 65, respectively. |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID Nos: 2/10, 18/26, |
| | 34/42, 50/58, 66/74, 82/90, 98/106, 1 14/122, 130/138, 146/154, |
| | 162/170, 178/186, 194/202, 210/202, 218/202, 226/202, 234/202, |
| | 242/202, 250/202, 258/202, 266/202, 274/202, 282/202, 290/202, |
| | 298/186, 306/186 and 314/186 of PCT Pub. WO2015112800A1. |
| PD1 | Anti-PD1 antibodies disclosed in U.S. Pat. No. 10,294,299 B2 |
| | as having the following SEQ ID NO. pairs for heavy and light |
| | chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb whose heavy |
| | and light chain sequences are disclosed as BAP049-Clone-E in |
| | U.S. Pat. No.: 9,683,048 B2. |
| | HC: SEQ ID NO: 91 of U.S. Pat. No.: 9,683,048 |
| | LC: SEQ ID NO: 72 of U.S. Pat. No.: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. 7,488,802 as |
| | having the following SEQ ID NO. pairs for heavy and light chain |
| | variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 7,521,051 |
| | as having the following SEQ ID NO. pairs for heavy and light |
| | chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 8,008,449 |
| | as having the following SEQ ID NO. pairs for heavy and light |
| | chain variable domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. 8,354,509 as |
| | having the following SEQ ID NO. pairs for heavy and light chain |
| | full-length domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. 8,168,757 as |
| | having the following SEQ ID NO. pairs for heavy and light chain |
| | variable domains: |
| | SEQ ID Nos. 4/5 |
| | SEQ ID Nos. 12/13 |
| | SEQ ID Nos. 18/19 |
| | SEQ ID Nos. 40/41 |
| | SEQ ID Nos. 47/48 |
| | SEQ ID Nos. 26/27 |
| | SEQ ID Nos. 34/35 |
| | SEQ ID Nos. 55/56 |
| | SEQ ID Nos. 67/68 |
| PD1 | Anti-PD1 antibodies described in PCT Pub. No. WO2004/004771 |
| PD1 | Anti-PD1 antibodies described in PCT Pub. No. WO2004/056875 |
| | as having the following SEQ ID NO. pairs for heavy and light |
| | chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. WO2004/072286 |
| PD1 | VH: SEQ ID NO: 25, 26, 27, 28, or 29 of US Pub. No. |
| | US2011/0271358 |
| | VL: SEQ ID NO: 30, 31, 32, or 33 of US Pub. No. |
| | US2011/0271358 |
| PD1 | SHR-1210 (Camrelizumab) described in PCT Publication No: |
| | WO 2015/085847 as having the following heavy and light |
| | chain variable domains: |
| | HC: SEQ ID NO: 9 |
| | LC: SEQ ID NO: 10 |
| PD1 | 4A11 described in U.S. Pat. No. 8,008,449 as having the |
| | following heavy and light chain variable domains: |
| | VH: SEQ ID NO: 5 |
| | VL: SEQ ID NO: 12 |
| PD1 | h1H3 Var 6 described in U.S. Pat. No. 9,205,148 as having |
| | the following heavy and light chain variable domains: |
| | VH: SEQ ID NO: 106 |
| | VL: SEQ ID NO: 98 |
| PD1 | PD1AB-6 described in U.S. Pat. No. 10,428,145 as having |
| | the following heavy and light chain variable domains: |
| | VH: SEQ ID NO: 13 |
| | VL: SEQ ID NO: 8 |
| PD1 | A1.5 described in U.S. Pat. No. 10,513,558 as having the |
| | following heavy and light chain variable domains: |
| | VH: SEQ ID NO: 21 |
| | VL: SEQ ID NO: 47 |
| PD1 | Anti-PD1 antibodies 244C8-2 and 388D4-3, described in |
| | U.S. Pat. No. 10,544,217 as having the following SEQ ID NO. |
| | pairs for heavy and light chain variable domains, respectively: |
| | SEQ ID NOs: 85/93 |
| | SEQ ID NOs: 88/96 |
| PD1 | Anti-PD1 antibodies 9B2C6C9 and 76G5B3, described in PCT |
| | Publication No. WO 2017/016497 as having the following SEQ |
| | ID NO. pairs for heavy and light chain variable domains, |
| | respectively: |
| | SEQ ID NOs: 1/5 |
| | SEQ ID NOs: 9/13 |
| PD1 | Anti-PD1 antibodies 1.153.7 hAb and 1.103.11 hAbv2 |
| | described in PCT Publication No. WO 2017/025051 as having |
| | the following SEQ ID NO. pairs for heavy and light chain |
| | variable domains, respectively: |
| | SEQ ID NOs: 61/63 |
| | SEQ ID NOs: 53/55 |
| PD1 | Clone 19 described in U.S. Pat. No. 8,927,697 as having |
| | the following heavy and light chain variable domains: |
| | VH: SEQ ID NO: 14 |
| | VL: SEQ ID NO: 12 |
| PD1 | PD1-0103-312 described in PCT Publication No. WO |
| | 2017/055443 as having the following heavy and light chain |
| | variable domains: |
| | VH: SEQ ID NO: 57 |
| | VL: SEQ ID NO: 58 |
| PD1 | Anti-PD1 antibodies h4B10-3 and 1353-G08 described in PCT |
| | Publication No. WO 2016/077397 as having the following SEQ |
| | ID NO. pairs for heavy and light chain variable domains, |
| | respectively: |
| | SEQ ID NOs: 264/288 |
| | SEQ ID NOs: 251/275 |
| PD1 | PD-1-27 described in PCT Publication No. WO 2016/210129 |
| | as having the following heavy and light chain variable |
| | domains: |
| | VH: SEQ ID NO: 51 |
| | VL: SEQ ID NO: 49 |
| PD1 | Anti-PD1 antibodies RG1H10 and RG1H10-16C described in |
| | PCT Publication No. WO 2014/194302 as having the following |
| | SEQ ID NO. pairs for heavy and light chain variable domains, |
| | respectively: |
| | SEQ ID NOs: 23/24 |
| | SEQ ID NOs: 40/24 |
| PD1 | HuEH12-VH4-VK3 described in U.S. Pat. No. 9,102,727 as |
| | having the following heavy and light chain variable domains: |
| | VH: SEQ ID NO: 28 |
| | VL: SEQ ID NO: 32 |
| PD1 | Clone 39 described in U.S. Pat. No. 10,066,013 as having |
| | the following heavy and light chain variable domains: |
| | VH: SEQ ID NO: 35 |
| | VL: SEQ ID NO: 34 |
| PD1 | tislelizumab |
| PD1 | retifanlimab |
| PD1 | balstilimab |
| PD1 | dostarlimab |
| PD1 | sasanlimab |
| PD1 | sintilimab |
| PD1 | cemiplimab |
| PDL1 | Durvalumab (MEDI4736) |
| | HC: SEQ ID NO: 26 of PCT application No. WO2020225552 |
| | LC: SEQ ID NO: 27 of PCT application No. WO2020225552 |
| PDL1 | Atezolizumab (Tecentriq, MPDL3280A, RG7446) |
| | HC: SEQ ID NO: 20 of U.S. Pat. No. 8,217,149 |
| | LC: SEQ ID NO: 21 of U.S. Pat. No. 8,217,149 |
| PDL1 | MDX 1105 (BMS-936559) |
| PDL1 | Anti-PDL1 antibodies described in U.S. Pat. No. 7,943,743 |
| | as having the following SEQ ID NO. pairs for heavy and light |
| | chain variable domains: |
| | SEQ ID Nos: 1/11 |
| | SEQ ID Nos: 2/12 |
| | SEQ ID Nos: 3/13 |
| | SEQ ID Nos: 4/14 |
| | SEQ ID Nos: 5/15 |
| | SEQ ID Nos: 6/16 |
| | SEQ ID Nos: 7/17 |
| | SEQ ID Nos: 8/18 |
| | SEQ ID Nos: 9/19 |
| | SEQ ID Nos: 10/20 |
| PDL1 | Avelumab, described in U.S. Pat. No.: 9,624,298 as |
| | having the following heavy and light chain variable domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID Nos: |
| | 52, 56, and 77 of PCT Pub. No. 2018/080812 |
| | CDR-L1, CDR-L2 and CDR-L3 sequences of SEQ ID Nos: 65, |
| | 42, and 71 of PCT Pub. No. 2018/080812 |
| PDL1 | KN035 (Envafolimab) is a nanobody described as Hu56V2 in |
| | U.S. Pat. No. 11,225,522 as having the VHH SEQ ID NO: 34 |
| PDL1 | LY3434172 |
| PDL1 | LY3300054 (lodapolimab) described in PCT Pub No: WO |
| | 2017/034916 as having the following heavy and light chain |
| | variable domains: |
| | HC: SEQ ID NO: 10 |
| | L: SEQ ID NO: 11 |
| PDL1 | LDP (lesabelimab, ADG104) described in CN Patent No: |
| | 114225023 as having the following heavy and light chain |
| | variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No.: 10,118,963 |
| | as having the following heavy and light chain variable domains: |
| | HC: SEQ ID NO: 1 |
| | LC: SEQ ID NO: 2 |
| PDL1 | BGB-A333 (garivulimab) described in U.S. Pat. No.: |
| | 11,512,132 as having the following heavy and light chain |
| | variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or 145 of |
| | U.S. Pat. No. 11,208,486. |
| | VL: SEQ ID NO: 24 or 125 of U.S. Pat. No. 11,208,486. |
| Phosphatidylserine | (bavituximab) |
| PSCA | GEM3PSCA |
| PSMA | huJ591 |
| PSMA | Anti-PSMA antibodies having VH and VL sequences having |
| | the amino acid sequences of any one of the following SEQ ID |
| | NO: pairs from WO 2017/023761A1: 2/1642; 10/1642; |
| | 18/1642; 26/1642; 34/1642; 42/1642; 50/1642; 58/1642; |
| | 66/1642; 74/1642; 82/1642; 90/1642; 98/1642; 106/1642; |
| | 114/1642; 122/130; and 138/146. |
| PSMA | An antibody such as: PSMA 3.7, PSMA 3.8, PSMA 3.9, PSMA |
| | 3.11, PSMA 5.4, PSMA 7.1, PSMA 7.3, PSMA 10.3, PSMA |
| | 1.8.3, PSMA A3.1.3, PSMA A3.3.1, Abgenix 4.248.2, Abgenix |
| | 4.360.3, Abgenix 4.7.1, Abgenix 4.4.1, Abgenix 4.177.3, |
| | Abgenix 4.16.1, Abgenix 4.22.3, Abgenix 4.28.3, Abgenix |
| | 4.40.2, Abgenix 4.48.3, Abgenix 4.49.1, Abgenix 4.209.3, |
| | Abgenix 4.219.3, Abgenix 4.288.1, Abgenix 4.333.1, Abgenix |
| | 4.54.1, Abgenix 4.153.1, Abgenix 4.232.3, Abgenix 4.292.3, |
| | Abgenix 4.304.1, Abgenix 4.78.1 and Abgenix 4.152.1 |
| | described in WO2003034903A2 |
| | A hybridoma cell line such as: PSMA 3.7 (PTA-3257), PSMA |
| | 3.8, PSMA 3.9 (PTA- 3258), PSMA 3.11 (PTA-3269), PSMA |
| | 5.4 (PTA-3268), PSMA 7.1 (PTA-3292), PSMA 7.3 (PTA- |
| | 3293), PSMA 10.3 (PTA-3247), PSMA 1.8.3 (PTA-3906), |
| | PSMA A3.1.3 (PTA- 3904), PSMA A3.3.1 (PTA-3905), |
| | Abgenix 4.248.2 (PTA-4427), Abgenix 4.360.3 (PTA- 4428), |
| | Abgenix 4.7.1 (PTA-4429), Abgenix 4.4.1 (PTA-4556), |
| | Abgenix 4.177.3 (PTA-4557), Abgenix 4.16.1 (PTA-4357), |
| | Abgenix 4.22.3 (PTA-4358), Abgenix 4.28.3 (PTA-4359), |
| | Abgenix 4.40.2 (PTA-4360), Abgenix 4.48.3 (PTA-4361), |
| | Abgenix 4.49.1 (PTA-4362), Abgenix 4.209.3 (PTA-4365), |
| | Abgenix 4.219.3 (PTA-4366), Abgenix 4.288.1 (PTA-4367), |
| | Abgenix 4.333.1 (PTA-4368), Abgenix 4.54.1 (PTA-4363), |
| | Abgenix 4.153.1 (PTA-4388), Abgenix 4.232.3 (PTA-4389), |
| | Abgenix 4.292.3 (PTA-4390), Abgenix 4.304.1 (PTA-4391), |
| | Abgenix 4.78.1 (PTA-4652), and Abgenix 4.152.1(PTA-4653) |
| | described in WO 2003/034903A2. |
| | VH of SEQ ID Nos: 2-7 described in WO 2003/034903A2 |
| | VL of SEQ ID Nos: 8-13 described in WO 2003/034903A2 |
| PSMA | VH: SEQ ID Nos: 225, 239, 253, 267, 281, 295, 309, 323, |
| | 337, 351, 365, 379, 393, 407, 421, 435, 449, 463, 477, 491, |
| | 505, 519, 533, 547, 561, 575, 589, 603 or 617 described in |
| | WO 2011/121110A1. |
| | VL SEQ ID Nos: 230, 244, 258, 272, 286, 300, 314, 328, 342, |
| | 356, 370, 384, 398, 412, 426, 440, 454, 468, 482, 496, 510, |
| | 524, 538, 552, 566, 580, 594, 608 or 622 described in WO |
| | 2011/121110A1. |
| | VH and VL SEQ ID Nos: 235, 249, 263, 277, 291, 305, 319, |
| | 333, 347, 361, 375, 389, 403, 417, 431, 445, 459, 473, 487, |
| | 501, 515, 529, 543, 557, 571, 585, 599, 613 or 627 described |
| | in WO 2011/121110A1. |
| PSMA | An anti-PSMA antibody having a VL amino acid sequence of |
| | any one of SEQ ID Nos: 229-312 of US 2022/0119525 A1 and |
| | a VH of SEQ ID NO: 217 of US 2022/0119525 A1. |
| PSMA | ES414 |
| PSMA | BAY2010112 (pasotuxizumab) |
| PSMA | CCW702 |
| PSMA | JNJ-63898081 |
| PSMA | CC-1 |
| PSMA | Acapatamab |
| PSMA | HPN424 |
| RAAG12 | RAV12 |
| RANKL | Prolia ™ (denosumab) |
| | VH: SEQ ID NO: 51 of US Patent Pub. 2017/0002060 |
| | VL: SEQ ID NO: 52 of US Patent Pub. 2017/0002060 |
| SLAMF7 | Empliciti ™ (elotuzumab) |
| SSTR2 | XmAb ®18087 |
| STEAP1 | VHCDR1 SEQ ID Nos: 14, 33, 182, 184 or 185 described in |
| | US20210179731A1. |
| | VHCDR2 SEQ ID Nos: 15, 21, 34, 182, 184 or 185 described |
| | in US20210179731A1. |
| | VHCDR3 SEQ ID Nos: 16 and 35 described in |
| | US20210179731A1. |
| | VH SEQ ID Nos: 182 or 184 described in US20210179731A1. |
| | VLCDR1 SEQ ID Nos: 11 or 30 described in |
| | US20210179731A1. |
| | VLCDR2 SEQ ID Nos: 12 or 31 described in |
| | US20210179731A1. |
| | VLCDR3 SEQ ID Nos: 13 or 32 described in |
| | US20210179731A1. |
| | VL SEQ ID Nos: 183 or 186 described in US20210179731A1. |
| STEAP1 | AMG509 |
| STEAP2 | Anti-STEAP 2 antibodies having CDR-H1, CDR-H2, CDR-H3, |
| | CDR-L1, CDR-L2 and CDR-L3 sequences selected from SEQ |
| | ID NOS: (1) 4-6-8-12-14-16; (2) 20-22-24-28-30-32; (3) 36-38- |
| | 40-44-46-48; (4) 52-54-56-60-62-64; (5) 68-70-72-60-62-64; |
| | (6) 76-78-80-60-62-64; (7) 84-86-88-60-62-64; (8) 92-94-96- |
| | 60-62-64; (9) 100-102-104-60-62-64; (10) 108-110-112-116- |
| | 118-120; (11) 124-126-128-132-134-136; (12) 140-142-144- |
| | 148-150-152; (13) 156-158-160-164-166-168; (14) 172-174- |
| | 176-180-182-184; (15) 188-190-192-196-198-200; (16) 204- |
| | 206-208-212-214-216; (17) 220-222-224-228-230-232; (18) |
| | 236-238-240-244-246-248; (19) 252-254-256-260-262-264; |
| | (20) 268-270-272-276-278-280; (21) 284-286-288-292-294- |
| | 296; (22) 300-302-304-308-310-312; (23) 316-318-320-324- |
| | 326-328; (24) 332-334-336-340-342-344; (25) 348-350-352- |
| | 356-358-360; (26) 364-366-368-372-374-376; and (27) 380- |
| | 382-384-388-390-392 of U.S. Pat. No. 10,772,972 B2. |
| | Anti-STEAP 2 antibodies having (a) a VH comprising the |
| | amino acid of any one of SEQ ID Nos: 2, 18, 34, 50, 66, 74, |
| | 82, 90, 98, 106, 122, 138, 154, 170, 186, 202, 218, 234, 250, |
| | 266, 282, 298, 314, 330, 346, 362, and 378 of U.S. Pat. No. |
| | 10,772,972 B2; and (b) a VL comprising the amino acid |
| | sequence of any one of SEQ ID Nos: 10; 26; 42; 58; 114; 130; |
| | 146; 162; 178; 194; 210; 226, 242; 258; 274; 290; 306; 322; |
| | 338; 354; 370; and 386 of U.S. Pat. No. 10,772,972 B2. |
| | Anti-STEAP 2 antibodies having a VH/VL pair comprising the |
| | amino acid sequences of any of the following pairs of SEQ ID |
| | Nos of U.S. Pat. No. 10,772,972 B2: 2/10; 18/26; 34/42; |
| | 50/58; 66/58; 74/58; 82/58; 90/58; 98/58; 106/114; 122/130; |
| | 138/146; 154/162; 170/178; 186/194; 202/210; 218/226; |
| | 234/242; 250/258; 266/274; 282/290; 298/306; 314/322; |
| | 330/338; 346/354; 362/370; and 378/386. |
| Syndecan-1 (CD 138) | The B-B4 antibody described in Wijdenes et al. (1996) Br. J. |
| | Haematol., 94: 318-323 |
| Syndecan-4 | The amino acid sequence of amino acids 93 and 121 of SEQ |
| | ID NO: 1 or the amino acid sequence of amino acids 92 and |
| | 122 of SEQ ID NO: 2 described in European Patent Pub. EP 2 |
| | 603 236. |
| TGFβ | GC1008 |
| TNFR | Enbrel ™ (etanercept) |
| TNFα | Remicade ™ (infliximab) |
| | VH: SEQ ID NO: 2 of Int. Patent Publication WO201/3087911 A1 |
| | VH: SEQ ID NO: 3 of Int. Patent Publication WO2013/ |
| | A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |
| XCR1 | Anti-XCR1 antibodies disclosed in U.S. Pat. No. 9,371,389 |
| | B2, including: |
| | The antibodies designated 2H6, 5G7, 11H2, HK1L2 |
| | and HK5L5 |
| | Antibodies having CDR-H1, CDR-H2 and CDR-H3 |
| | sequences of SEQ ID Nos: 53-55 and CDR-L1, CDR- |
| | L2 and CDR-L3 sequences of SEQ ID Nos: 56-58. |
| | Antibodies having CDR-H1, CDR-H2 and CDR-H3 |
| | sequences of SEQ ID Nos: 41-43 and CDR-L1, CDR- |
| | L2 and CDR-L3 sequences of SEQ ID Nos: 44-46. |

In some aspects, the tumor antigen targeting moiety competes with an antibody set forth in Table T-3 for binding to the tumor antigen. In further aspects, the tumor antigen targeting moiety comprises CDRs having CDR sequences of an anti-tumor antigen antibody set forth in Table T-3. In some embodiments, the tumor antigen targeting moiety comprises all 6 CDR sequences of the anti-tumor antigen antibody set forth in Table T-3. In other embodiments, the tumor antigen targeting moiety comprises at least the heavy chain CDR sequences (CDR-H1, CDR-H2, CDR-H3 and the light chain CDR sequences of a universal light chain. In further aspects, a tumor antigen targeting moiety comprises a VH comprising the amino acid sequence of the VH of an anti-tumor antigen antibody set forth in Table T-3. In some embodiments, the tumor antigen targeting moiety further comprises a VL comprising the amino acid sequence of the VL of the anti-tumor antigen antibody set forth in Table T-3. In other embodiments, the tumor antigen targeting moiety further comprises a universal light chain VL sequence.

In some embodiments, the targeting moieties target the exemplary target molecules set forth in Table T-4 below, which provides references to exemplary single domain antibodies or antibody sequences upon which the targeting moiety can be based.

TABLE T-4
Exemplary Single Domain Antibody (sdAb) Amino Acid Sequences

| | | | SEQ |
|---|---|---|---|
| | | | ID |
| Target | Reference | Sequence | NO |
| B7H3 | SEQ ID NO: 1 of | HVQLVESGGGLVQPGRSLRLSCAASGFTFSSYWMYWV | 7 |
| | PCT Publication | RQTPGKGLEWVSTINRDGSATWYADSVKGRFTISRDN | |
| | No. WO | AKNTGYLQMNSLEPDDTAVYYCVSDPDNYSSDEMVPY | |
| | 2021/247794 A2 | WGQGTQVTVSS | |
| B7H3 | SEQ ID NO:2 of | QVQLVESGGGLVQPGGSLRLSCAASGFTFSSYWMYWV | 8 |
| | PCT Publication | RQTPGKGLEWVSTINRDGSATWYADSVKGRFTISRDN | |
| | No. WO | AKNTGYLQMNSLKPDDTAVYYCVSDPDNYSSDEMVPY | |
| | 2021/247794 A2 | WGQGTQVTVSS | |
| B7H3 | SEQ ID NO:3 of | XVQLVESGGGLVQPGXSLRLSCAASGFTFSSYWMYWV | 9 |
| | PCT Publication | RQTPGKGLEWVSTINRDGSATWYADSVKGRFTISRDN | |
| | No. WO | AKNTGYLQMNSLXPDDTAVYYCVSDPDNYSSDEMVPY | |
| | 2021/247794 A2 | WGQGTQVTVSS | |
| CA9 | SEQ ID NO:1 of | QVQLVESGGGLVQAGGSLRLSCAASGFTFDDWAIGWF | 10 |
| | PCT Publication | RQAPGKEREGVSCISKRHGTTHYADSVKGRFTISSDN | |
| | No. WO | AKNTVYLRMNGLKPEDTAVYYCAASSWGSCTVATMRD | |
| | 2022/157714 A1 | VDRYDYDYWGQGTQVTVSS | |
| CEACAM | Jancewicz et al, | QVKLEESGGGLVQAGGSLRLSCRTSGRINSVYTMGWF | 11 |
| | 2024, Cancer | RQAPGKEREFVAQIMWGAGTNTHYADSVKGRFTISRD | |
| | Immunol | SAESTVYLQMNSLKPEDTAVYYCAANRGIPIAGRQYD | |
| | Immunother. | YWGQGTQVTVSS | |
| | 73(2):30 | | |
| EpCAM | SEQ ID NO: 1 of | DVQLVESGGGSVQSGGSLRLSCAASGYTYRRYYMGWF | 12 |
| | PCT Publication | RQAPGEQREGVAVINNDGRTNYADSVKGRFRISRDNA | |
| | No. WO | ENTLHLEMNSLKPEDTAMYYCAATGNILPPMTAVPPL | |
| | 2023/044991 A1 | GRQWYPYWGRGTLVTVSS | |
| EpCAM | SEQ ID NO:2 of | HVQLVESGGGSVQSGGSLRLSCAASGYAVKNCMGWFR | 13 |
| | PCT Publication | QAPGKEREGVAVINRNGITTYADSVKGRFTISQDKDK | |
| | No. WO | NTLDLQMNSLKPEDTAMYYCAATPTLLTIPARFLCDV | |
| | 2023/044991 A1 | RNPSGFTDWGQGTLVTVSS | |
| EpCAM | SEQ ID NO:3 of | QVQLVESGGGSVQAGGSLRLSCVVSAYSAYTYKTMCM | 14 |
| | PCT Publication | GWFRQAPGKEREGVAAIYRGGLNTYYADSVKGRFIIS | |
| | No. WO | RDNAESTMYLQMNSLKPEDTAMYYCAADWLRGDDCNI | |
| | 2023/044991 A1 | GANFDYWGQGTQVTVSS | |
| EpCAM | SEQ ID NO:4 of | QVQLVESGGGSVQAGGSLRLSCVATGFTISRKCMGWF | 15 |
| | PCT Publication | REAPGKKREVIATINTGSSSPYYADGVKGRFTISQDN | |
| | No. WO | AKNTVYLQMNSLKPEDTAMYYCAATKGVVVGTGYCGG | |
| | 2023/044991 A1 | PYVERPNSAYWGQGTQVTVSS | |
| EpCAM | SEQ ID NO:5 of | DVQLVESGGGSVQAGRSLRLSCELSDYTWSTVCMGWF | 16 |
| | PCT Publication | RQAPGKEREGVAVIYTRSGGTTYADSAKGRFTISRDN | |
| | No. WO | AKDTLYLQMDSLKPEDTAMYYCAAGPLYDGRCTYRSP | |
| | 2023/044991 A1 | AFHYWGQGTQVTVSS | |
| EpCAM | SEQ ID NO:6 of | DVQLVESGGGSAQAGGSLRLSCAASGPTSSLRTMGWF | 17 |
| | PCT Publication | RQASGKERERVAVIWDGRTTDYDDSVQDRFTISQDNA | |
| | No. WO | KSTVYLQMNTLKPEDTAMYYCAASPRIVPFASTYFQH | |
| | 2023/044991 A1 | WGQGTQVTVSS | |
| EpCAM | SEQ ID NO:7 of | HVQLVESGGGSVQAGGSLKLSCAASGSIFSGSIFSRC | 18 |
| | PCT Publication | GMRWYRQAPGKERELVSSTSKDGFTSYTDSVKGRFTI | |
| | No. WO | SQDNANNTLYLQMSSLKTEDTAVYSCAAICAVGGYSL | |
| | 2023/044991 A1 | STYTYWGQGTQVTVSS | |
| EpCAM | SEQ ID NO:8 of | EVLVESGGDSVQAGGSLRLSCAASGYSPGSYCMGWF | 19 |
| | PCT Publication | RQAPGKERERVAIIESRGTVTYVDSVKGRFTISKDNA | |
| | No. WO | KNTLYLQMNSLKPEDTAMYYCAASRPWSGVRCLHDKY | |
| | 2023/044991 A1 | DYWGQGTQVTVSS | |
| EpCAM | SEQ ID NO:9 of | HVQLVESGGGSVQSGGSLRLSCAVSGYAYSSLAWFRQ | 39 |
| | PCT Publication | APGKEREGVAALLTAIGGPTRTTYADSVKGRLAISQD | |
| | No. WO | HAKNTLYLQMSSLKPEDTAMYYCAAGRPAGTPRWLLL | |
| | 2023/044991 A1 | APRDYNYWGQGTQVTVSS | |
| HER2 | SEQ ID NO:7 of | QVQLQESGGGSVQAGGSLKLTCAASGYIFNSCGMGWY | 40 |
| | PCT Publication | RQSPGRERELVSRISGDGDTWHKESVKGRFTISQDNV | |
| | No. WO | KKTLYLQMNSLKPEDTAVYFCAVCYNLETYWGQGTQV | |
| | 2016/016021 A1 | TVSS | |
| HER2 | SEQ ID NO:8 of | QVQLQESGGGLVQPGGSLRLSCAASGFIFSNDAMTWV | 41 |
| | PCT Publication | RQAPGKGLEWVSSINWSGTHTNYADSVKGRFTISRDN | |
| | No. WO | AKRTLYLQMNSLKDEDTALYYCVTGYGVTKTPTGQGT | |
| | 2016/016021 A1 | QVTVSS | |
| HER3 | SEQ ID NO:265 of | QVQLVQSGGGLVQAGGSLSLSCAFSGRTFSMYTMGWF | 42 |
| | PCT Publication | RQAPGKEREFVAANRGRGLSPDIADSVNGRFTISRDN | |
| | No. WO | AKNTLYLQMDSLKPEDTAVYYCAADLQYGSSWPQRSS | |
| | 2021/188736 A1 | AEYDYWGQGTTVTVSS | |
| MSLN | SEQ ID NO: 1 of | QVQLVQSGGGLVHPGGSLRLSCAASGIDLSLYRMRWY | 43 |
| | U.S. Publication | RQAPGKERDLVALITDDGTSYYEDSVKGRFTITRDNP | |
| | No. US | SNKVFLQMNSLKPEDTAVYYCNAETPLSPVNYWGQGT | |
| | 2018/0002439 A1 | QVTVS | |
| MSLN | SEQ ID NO:2 of | QVQLVQSGGGLVQAGGSLRLSCAPSGSIFGIRTMDWY | 44 |
| | U.S. Publication | RQAPGKERELVARITMDGRVFHADSVKGRFSGSRDGA | |
| | No. US | SNAVYLQMNSLKPDDTAVYYCRYSGLTSREDYWGPGT | |
| | 2018/0002439 A1 | QVTVSS | |
| MSLN | SEQ ID NO:97 of | QVQLVQSGGGLVHPGGSLRLSCAASGIDLSLYRMRWY | 43 |
| | PCT Publication | RQAPGKERDLVALITDDGTSYYEDSVKGRFTITRDNP | |
| | No. WO | SNKVFLQMNSLKPEDTAVYYCNAETPLSPVNYWGQGT | |
| | 2020/023888A2 | QVTVS | |
| MSLN | SEQ ID NO:98 of | QVQLVQSGGGLVQAGGSLRLSCAPSGSIFGIRTMDWY | 44 |
| | PCT Publication | RQAPGKERELVARITMDGRVFHADSVKGRFSGSRDGA | |
| | No. WO | SNAVYLQMNSLKPDDTAVYYCRYSGLTSREDYWGPGT | |
| | 2020/023888A2 | QVTVSS | |
| MUC16 | SEQ ID NO: 15 of | QVQLQESGGGLVQAGGSLRLSCAASGRTVSSLFMGWF | 45 |
| | PCT Publication | RQAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO:20 of | QVQLQESGGGLVQAGDSLRLSCAASGRAVSSLFMGWF | 46 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO:25 of | QVQLQESGGGLVQAGDSLRLSCAASGRTVSSLFMGWF | 47 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO:30 of | QVQLQESGGGLVQPGDSMRLSCAAEGDSLDGYVVGWF | 48 |
| | PCT Publication | RQAPGKERQGVSSISGDGSMRYVADSVKGRFTISRDN | |
| | No. WO | AKNTVYLQMIDLKPEDTGVYYCAADPPTWDYWGQGTQ | |
| | 2020/023888 A2 | VTVSS | |
| MUC16 | SEQ ID NO:35 of | QVQLQESGGGLVQPGGSLRLSCAASGRTVSSLFMGWF | 49 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO:40 of | QVQLQESGGGLVQAGESLRLSCAASGRTVSSLFMGWF | 50 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| PSMA | Xing et al., 2021 | EVLVESGGGLVQPGGSLTLSCAASREMISEYSMHWV | 51 |
| | Int. J Mol Sci. | RQAPGKGLEWVSTINPAGTTDYAESVKGRFTISRDNA | |
| | 22(11):5501 | KNTLYLQMNSLKPEDTAVYY | |
| | | CDGYGYRGQGTQVTVSS | |
| PSMA | SEQ ID NO:38 of | QLQLVESGGGLVHAGGSLRLSCAASGSTFSINAIGWY | 52 |
| | PCT Publication | RQAPGKRELVAALSSGGSKNYADSVKGRFTISRDNA | |
| | No. WO | KNTVYLQMNRLKPEDTAVYYCNAEIYYSDGVDDGYRG | |
| | 2022/234473 A1 | MDYWGKGTQVTVSS | |
| PSMA | SEQ ID NO:42 of | EVQVVESGGGLVQTGGSLRLSCAASGPPLSSYAVAWF | 53 |
| | PCT Publication | RQTPGKEREFVAAISWSGSNTYYADSVKGRFTISKDN | |
| | No. WO | AKNTVLVYLQMNSLKPEDTAVYYCAADRRGGPLSDYE | |
| | 2022/234473 A1 | WEDEYADWGQGTQVTVSS | |

In some aspects, the tumor antigen targeting moiety competes with an antibody set forth above in Table T-4 for binding to the target molecule. In further aspects, the tumor antigen targeting moiety comprises CDRs having CDR sequences of an antibody set forth in Table T-4. In some embodiments, the targeting moiety comprises all 3 CDR sequences of the antibody set forth in Table T-4. In further aspects, a targeting moiety comprises a VH (e.g., a VHH) comprising the amino acid sequence of the VH of an antibody set forth in Table T-4.

Additional tumor antigens that can be targeted by a tumor antigen targeting moiety are disclosed in, e.g., Hafeez et al., 2020, Molecules 25:4764, doi:10.3390/molecules25204764, particularly in Table 1. Table 1 of Hafeez et al. is incorporated by reference in its entirety here.

### 6.4.3. Low-Density Antigen Targeting Moieties

Many cancers are characterized by low-density antigens, for example by virtue of the cancer-specific antigen being present only as an HLA-bound peptide. Targeting of such cancers is particularly challenging due to the low expression of the target antigen, making targeting difficult using traditional bispecific antibodies having a single T cell antigen (e.g., CD3) targeting moiety and a single target antigen targeting moiety. Accordingly, in some embodiments, an MBM of the present disclosure comprises at least one targeting moiety that binds specifically to a polypeptide antigen present on the surface of a cell or population of cells at an average of no more than 5000 copies per cell (a “low-density antigen”), referred to herein as “low-density antigen targeting moieties.” In general, a low-density antigen targeting moiety of the disclosure specifically binds to a low-density antigen present at no more than 5000, 4000, 3000, 2000, 1000, 500, 400, 300, 200, 100, 50, 40, 30, 20, or 10 copies per cell, or less. In some embodiments, the low-density antigen is present at between 10 and 100, between 50 and 200, between 100 and 300, between 200 and 500, between 300 and 750, between 500 and 1000, between 750 and 1500, or between 1000 and 2000 copies per cell. In particular embodiments, the low-density antigen is present at no more than 200, no more than 100, or no more than 50 copies per cell.

As described herein, MBMs of the disclosure comprising two low-density antigen targeting moieties and two TCA (e.g., CD3) targeting moieties are capable of significantly enhancing T cell activation relative to a traditional bispecific antibody targeting the same low-density antigen and CD3. Without being bound by theory, it is understood that the inclusion of two TCA targeting moieties and two low-density antigen targeting moieties, arranged as in the MBMs described herein (e.g., as depicted in FIG. 1A, 1B, or 1C), confers a particular advantage in activating T cells relative to other formats.

In some embodiments, the low-density antigen targeting moiety is a tumor antigen targeting moiety, e.g., as described in Section 6.4.1.1.2. In some embodiments, the low-density antigen targeting moiety is an HLA-bound peptide antigen targeting moiety, e.g., as described in Section 6.4.4.

### 6.4.4. HLA-Bound Peptide Antigen Targeting Moieties

Many cancers are characterized by HLA-bound peptide antigens which are uniquely presented by the cancer cells. These include so-called “neoantigens”, which are generally either mutations relative to a wild type allele or result from the expression of a new open reading frame in cancer cells (as shown in Table 1 of Fritsch et al., 2014, Cancer Immunol Res 2:522-529, incorporated by reference herein in its entirety), as well as peptides from viral-associated cancers, such as HPV. In addition, viral infections generally result in HLA presentation of peptides from intracellular processing of viral proteins, serving as a unique marker of infected cells relative to uninfected cells. However, such HLA-bound peptides are often expressed on the surface of the target cell (e.g., cancer cell, virally infected cell) at low copy number relative to other cell surface proteins, making targeting via traditional bispecific antibodies difficult. Accordingly, in some embodiments, an MBM of the present disclosure comprises at least one targeting moiety that binds specifically to an HLA-bound peptide antigen.

As described herein, MBMs comprising two HLA-bound peptide antigen targeting moieties and two TCA (e.g., CD3) targeting moieties are capable of significantly enhancing T cell activation relative to a traditional bispecific antibody targeting the same low-density antigen and CD3. Without being bound by theory, it is understood that the inclusion of two TCA targeting moieties and two HLA-bound peptide antigen targeting moieties, arranged as in the MBMs described herein (e.g., as depicted in FIG. 1A, 1B, or 1C), confers a particular advantage in activating T cells relative to other formats.

In some embodiments, the HLA-bound peptide antigen targeting moiety is a tumor antigen targeting moiety, e.g., as described in Section 6.4.1.1.2. In some embodiments, the HLA-bound peptide antigen targeting moiety is a low-density antigen targeting moiety, e.g., as described in Section 6.4.3.

Exemplary HLA-bound peptide antigens which may be targeted by a targeting moiety of the disclosure are set forth in Table P-1, below.

TABLE P-1

| | HLA | Peptide | SEQ | |
|---|---|---|---|---|
| Peptide | allele | sequence | ID NO | Type |
| KRAS(6-14)G12V | A02:01 | LVVVGAVGV | 54 | Cancer antigen |
| KRAS(5-14)G12D | A02:01 | KLVVVGADGV | 55 | Cancer antigen |
| KRAS(5-14)G12V | A02:01 | KLVVVGAVGV | 56 | Cancer antigen |
| KRAS(10-18)G12D | C08:02 | GADGVGKSA | 57 | Cancer antigen |
| KRAS(10-19)G12D | C08:02 | GADGVGKSAL | 58 | Cancer antigen |
| KRAS(8-16)G12D | A11:01 | VVGADGVGK | 59 | Cancer antigen |
| KRAS(8-16)G12V | A11:01 | VVGAVGVGK | 60 | Cancer antigen |
| KRAS(7-16)G12D | A11:01 | VVVGADGVGK | 61 | Cancer antigen |
| KRAS(7-16)G12V | A11:01 | VVVGAVGVGK | 62 | Cancer antigen |
| MAGE-A3(168-176) | A01:01 | EVDPIGHLY | 63 | Cancer antigen |
| MAGE-A3(271-279) | A02:01 | FLWGPRALV | 64 | Cancer antigen |
| MAGE-A3(108-116) | A02:01 | ALSRKVAEL | 65 | Cancer antigen |
| MAGE-A10(137-145) | A02:01 | KVTDLVQFL | 66 | Cancer antigen |
| MAGE-A10(254-262) | A02:01 | GLYDGMEHL | 67 | Cancer antigen |
| MAGE-A10(310-318) | A02:01 | SLLKFLAKV | 68 | Cancer antigen |
| WT-1(10-18) | A02:01 | ALLPAVPSL | 69 | Cancer antigen |
| PRAME(312-320) | A02:01 | RLDQLLRHV | 70 | Cancer antigen |
| PRAME(425-433) | A02:01 | SLLQHLIGL | 71 | Cancer antigen |
| PSA(79-89) | A02:01 | SLFHPEDTGQV | 72 | Cancer antigen |
| PSA(159-169) | A11:01 | SIEPEEFLTPK | 73 | Cancer antigen |
| PSA(241-249) | A11:01 | SLYTKVVHY | 74 | Cancer antigen |
| EBNA-1(562-570) | A02:01 | FMVFLQTHI | 75 | Cancer antigen |
| LMP2(426-434) | A02:01 | CLGGLLTMV | 76 | Cancer antigen |
| DNTT(475-483) | A02:01 | ALYDKTKRI | 77 | Cancer antigen |
| DNTT(250-258) | A02:01 | KLFTSVFGV | 78 | Cancer antigen |
| MPO(132-140) | A02:01 | SLWRRPFNV | 79 | Cancer antigen |
| MPO(549-557) | A02:01 | GLMATPAKL | 80 | Cancer antigen |
| MPO(571-579) | A02:01 | RLFEQVMRI | 81 | Cancer antigen |
| AFP(58-68) | A11:01 | ATYKEVSKMVK | 82 | Cancer antigen |
| AFP(173-181) | A24:02 | LYAPTILLW | 83 | Cancer antigen |
| IGF2BP3(552-560) | A02:01 | KIQEILTQV | 84 | Cancer antigen |
| KLK4(215-223) | A24:02 | GYLQGLVSF | 85 | Cancer antigen |
| KLK4(105-113) | B07:02 | HPEYNRPLL | 86 | Cancer antigen |
| ACP3(PAP)(112-120) | A02:01 | TLMSAMTNL | 87 | Cancer antigen |
| KLK3(PSA)(82-90) | HLA-B | HPEDTGQVF | 88 | Cancer antigen |
| BALF-4(276-284) | A02:01 | FLDKGTYTL | 89 | Cancer antigen |
| CEA(571-579) | A02:01 | YLSGANLNL | 90 | Cancer antigen |
| Her2/neu(369-377; V2v9) | A02:01 | KIFGSLAFL | 91 | Cancer antigen |
| LMP1(125-133) | A02:01 | YLLEMLWRL | 192 | Cancer antigen |
| MAGE-A3(112-120) | A02:01 | KVAELVHFL | 93 | Cancer antigen |
| MAGE-A4(230-239) | A02:01 | GVYDGREHTV | 94 | Cancer antigen |
| MAGE-A4(286-294) | A02:01 | KVLEHVVRV | 95 | Cancer antigen |
| NY-ESO-1(157-165, C165V) | A02:01 | SLLMWITQV | 96 | Cancer antigen |
| NY-ESO-1(157-165, C165A) | A02:01 | SLLMWITQA | 97 | Cancer antigen |
| p53(264-272) | A02:01 | LLGRNSFEV | 98 | Cancer antigen |
| PSMA(4-12) | A02:01 | LLHETDSAV | 99 | Cancer antigen |
| Survivin(96-104) | A02:01 | LTLGEFLKL | 100 | Cancer antigen |
| Tyrosinase(369-377, 371D) | A02:01 | YMDGTMSQV | 101 | Cancer antigen |
| WT1(126-134) | A02:01 | RMFPNAPYL | 102 | Cancer antigen |
| HIVP17(77-85) | A02:01 | SLYNTVATL | 103 | Viral antigen |
| HIVRT(896-904) | A02:01 | ILKEPVHGV | 104 | Viral antigen |
| HIVGAG(41-49) | HLA-E | AISPRTLNA | 105 | Viral antigen |
| ENV(183-191) | A02:01 | FLLTRILTI | 106 | Viral antigen |
| HBVPol(616-626 variant5) | A11:01 | GTLPQDHIVQK | 107 | Viral antigen |
| HBVCore(18-27) | A02:01 | FLPSDFFPSV | 108 | Viral antigen |
| HPVE7(11-20) | A02:01 | YMLDLQPETT | 109 | Cancer antigen; |
| | | | | Viral antigen |
| HPVE7(11-19) | A02:01 | YMLDLQPET | 110 | Cancer antigen; |
| | | | | Viral antigen |
| HPVE7(82-90) | A02:01 | LLMGTLGIV | 111 | Cancer antigen; |
| | | | | Viral antigen |

Example HLA-bound peptide antigen targeting moieties, including antibodies (e.g., multispecific antibodies) and T-cell receptors (TCRs), which may be incorporated into an MBM disclosed herein are set forth in Table P-2, below.

TABLE P-2
Example HLA-bound Peptide Antigen Targeting Moieties

| Target (HLA- | |
|---|---|
| bound peptide) | Antibody Name and/or Binding Sequences |
| MAGE-A4 286- | Anti-MAGE-A4 antibody sequences described in PCT Pub. No. WO |
| 294 | 2023/183758 A2 as having the following SEQ ID NO. pairs for heavy |
| | and light chain variable domains: |
| | SEQ ID NOs. 172/189 |
| | SEQ ID NOs. 172/180 |
| | SEQ ID NOs. 191/199 |
| NY-ESO-1 | Anti-HLA-A2: NY-ESO-1 amino acid sequences described in PCT Pub. |
| | No. WO 2021/003357 A1 as having the following SEQ ID NO. pairs for |
| | heavy and light chain variable domains: |
| | SEQ ID NOs. 2/10 |
| | SEQ ID NOs. 22/30 |
| | SEQ ID NOs. 42/50 |
| | SEQ ID NOs. 62/70 |
| | SEQ ID NOs. 82/90 |
| | SEQ ID NOs. 102/110 |
| | SEQ ID NOs. 122/130 |
| | SEQ ID NOs. 142/150 |
| | SEQ ID NOs. 162/170 |
| | SEQ ID NOs. 180/186 |
| | SEQ ID NOs. 196/203 |
| | SEQ ID NOs. 211/219 |
| | SEQ ID NOs. 230/238 |
| | SEQ ID NOs. 250/258 |
| PRAME (312- | TCR-alpha and TCR-beta CDR amino acid sequences described in |
| 320) | US. Pub. No. 2023/0060095 A1 that were identified with PRAME (312- |
| | 320) |
| | TCR-alpha CDR1 of SEQ ID NOs. 1, 7, 13, 19, 25, 31, 37, 43, 49, 55, |
| | 61, 67, 73, 79, 85, 91, 97, 103 |
| | TCR-alpha CDR2 of SEQ ID NOs. 2, 8, 14, 20, 26, 32, 38, 44, 50, 56, |
| | 62, 68, 74, 80, 86, 92, 98, 104 |
| | TCR-alpha CDR3 of SEQ ID NOs. 3, 9, 15, 21, 27, 33, 39, 45, 51, 57, |
| | 63, 69, 75, 81, 87, 93, 99, 105 |
| | TCR-beta CDR1 of SEQ ID NOs. 4, 10, 16, 22, 28, 34, 40, 46, 52, 58, |
| | 64, 70, 76, 82, 88, 94, 100, 106 |
| | TCR-beta CDR2 of SEQ ID NOs. 5, 11, 17, 23, 29, 35, 41, 47, 53, 59, |
| | 65, 71, 77, 83, 89, 95, 101, 107 |
| | TCR-beta CDR3 of SEQ ID NOs. 6, 12, 18, 24, 30, 36, 42, 48, 54, 60, |
| | 66, 72, 78, 84, 90, 96, 102, 108 |
| PRAME (425- | TCR-alpha and TCR-beta CDR amino acid sequences described in |
| 433) | US. Pub. No. 2023/0060095 A1 that were identified with PRAME (425- |
| | 433) |
| | TCR-alpha CDR1 of SEQ ID NOs. 289, 295, 301, 307, 313, 319, 325, |
| | 331, 337, 343, 349, 355, 361, 367, 373, 379, 385, 391, 397, 403, 409, |
| | 415, 421, 427, 433, 439, 445, 451, 457, 463, 469, 475, 481, 487, 493, |
| | 499, 505, 511, 517, 523 |
| | TCR-alpha CDR2 of SEQ ID NOs. 290, 296, 302, 308, 314, 320, 326, |
| | 332, 338, 344, 350, 356, 362, 368, 374, 380, 386, 392, 398, 404, 410, |
| | 416, 422, 428, 434, 440, 446, 452, 458, 464, 470, 476, 482, 488, 494, |
| | 500, 506, 512, 518, 524 |
| | TCR-alpha CDR3 of SEQ ID NOs. 291, 297, 303, 309, 315, 321, 327, |
| | 333, 339, 345, 351, 357, 363, 369, 375, 381, 387, 393, 399, 405, 411, |
| | 417, 423, 429, 435, 441, 447, 453, 459, 465, 471, 477, 483, 489, 495, |
| | 501, 507, 513, 519, 525 |
| | TCR-beta CDR1 of SEQ ID NOs. 292, 298, 304, 310, 316, 322, 328, |
| | 334, 340, 346, 352, 358, 364, 370, 376, 382, 388, 394, 400, 406, 412, |
| | 418, 424, 430, 436, 442, 448, 454, 460, 466, 472, 478, 484, 490, 496, |
| | 502, 508, 514, 520, 526 |
| | TCR-beta CDR2 of SEQ ID NOs. 293, 299, 305, 311, 317, 323, 329, |
| | 335, 341, 347, 353, 359, 365, 371, 377, 383, 389, 395, 401, 407, 413, |
| | 419, 425, 431, 437, 443, 449, 455, 461, 467, 473, 479, 485, 491, 497, |
| | 503, 509, 515, 521, 527 |
| | TCR-beta CDR3 of SEQ ID NOs. 294, 300, 306, 312, 318, 324, 330, |
| | 336, 342, 348, 354, 360, 366, 372, 378, 384, 390, 396, 402, 408, 414, |
| | 420, 426, 432, 438, 444, 450, 456, 462, 468, 474, 480, 486, 492, 498, |
| | 504, 510, 516, 522, 528 |
| MAGE-A4 (286- | Anti-MAGE-A4(286-294) VH and VL amino acid sequences described |
| 294) | in US Pub. No. 2022/0031748 A1 |
| | VH: SEQ ID NO: 21 |
| | VL: SEQ ID NO: 17 |
| NY-ESO (156- | Anti-NY-ESO (156-165) VH and VL amino acid sequences described |
| 165) | in US Pub. No. 2022/0031748 A1 |
| | VH: SEQ ID NO: 22 |
| | VL: SEQ ID NO: 18 |
| Tyrosinase D11 | Anti-Tyr (369-377) VH and VL amino acid sequences described in US |
| (369-377) | Pub. No. 2022/0031748 A1 |
| | VH: SEQ ID NO: 23 |
| | VL: SEQ ID NO: 19 |
| MAGE-A4 (230- | Anti-MAGE-A4 (230-239) VH and VL amino acid sequences described |
| 239) | in US Pub. No. 2022/0031748 A1 as having the following SEQ ID NO. |
| | pairs for heavy and light chain variable domains: |
| | SEQ ID NOs: 32/31 |
| | SEQ ID NOs: 38/37 |
| MAGE-A4 (230- | Anti-MAGE-A4 (230-239) VH and VL amino acid sequences described |
| 239) | in U.S. Pat. No. 11,497,768 |
| | VH: SEQ ID NO: 36 |
| | VL: SEQ ID NO: 38 |

In some aspects, the HLA-bound peptide antigen targeting moiety competes with an antibody or TCR set forth in Table P-2 for binding to the HLA-bound peptide antigen. In further aspects, the HLA-bound peptide antigen targeting moiety comprises CDRs having CDR sequences of an antibody or TCR set forth in Table P-2. In some embodiments, the HLA-bound peptide antigen targeting moiety comprises all 6 CDR sequences of an antibody or TCR set forth in Table P-2. In other embodiments, the HLA-bound peptide antigen targeting moiety comprises at least the heavy chain CDR sequences (CDR-H1, CDR-H2, CDR-H3 and the light chain CDR sequences of a universal light chain. In further aspects, the HLA-bound peptide antigen targeting moiety comprises a VH comprising the amino acid sequence of the VH of an antibody or TCR set forth in Table P-2. In some embodiments, the HLA-bound peptide antigen targeting moiety further comprises a VL comprising the amino acid sequence of the VL of an antibody or TCR set forth in Table P-2. In other embodiments, the tumor antigen targeting moiety further comprises a universal light chain VL sequence.

Example single domain HLA-bound peptide antigen targeting moieties, which may be incorporated into an MBM disclosed herein are set forth in Table P-3, below.

TABLE P-3
Example Single Domain HLA-bound Peptide Antigen Targeting Moieties

| Target (HLA- | |
|---|---|
| bound peptide) | Antibody Name and/or Binding Sequences |
| MAGE-A4 230- | Anti-MAGE-A4 single domain antibody sequences described in US |
| 239 | Pub. No. 2022/0380472 A1 as having the following SEQ ID NOs.: 190, |
| | 194, 198, 202, 205, 209, 213, 217, 221, 225, 229, 233, 237, 241, 245, |
| | 249, 253, 257, and 261 (see Table 8 of US Pub. No. 2022/0380472 |
| | A1, incorporated herein by reference). |
| HPV E6 | Anti-HLA antibody single domain antibody sequences described in |
| | PCT Pub. No. 2024/050399 A1 as having the SEQ ID NOs. 1 and 2 |
| | (see pages 20-21 of PCT Pub. No. 2024/050399 A1, incorporated |
| | herein by reference). |
| HPV E7 | Anti-HLA antibody single domain antibody sequences described in |
| | PCT Pub. No. 2024/050399 A1 as having the following SEQ ID |
| | NOs.: 3, 4, and 5 (see pages 20-21 of PCT Pub. No. 2024/050399 A1, |
| | incorporated herein by reference). |

In some aspects, the HLA-bound peptide antigen targeting moiety competes with a single domain antibody set forth in Table P-3 for binding to the HLA-bound peptide antigen. In further aspects, the HLA-bound peptide antigen targeting moiety comprises CDRs having CDR sequences of a single domain antibody set forth in Table P-3. In some embodiments, the HLA-bound peptide antigen targeting moiety comprises all 3 CDR sequences of a single domain antibody set forth in Table P-3. In further aspects, the HLA-bound peptide antigen targeting moiety comprises the amino acid sequence of the VH of a single domain antibody set forth in Table P-3.

### 6.5. Linkers

In certain aspects, the present disclosure provides recombinant polypeptides in which two or more components of the recombinant polypeptide are connected to one another by a linker (also “peptide linker”). By way of example and not limitation, linkers can be used to connect (a) a targeting moiety and an Fc domain; (b) a first targeting moiety and a second targeting moiety (e.g. a Fab and an scFv, or a first scFv and a second scFv); or (c) different domains within a targeting moiety (e.g., the VH and VL domains in an scFv).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the recombinant polypeptides of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS (SEQ ID NO: 112) or SGn (SEQ ID NO: 113), where n is an integer from 1 to 10, e.g., 1, 2, 3, 4, 5, 6, 7, 8, 9 or 10. In one embodiment, the linker is or comprises a monomer or multimer of repeat of G₄S (SEQ ID NO: 114) e.g., (GGGGS)ₙ(SEQ ID NO: 115), where n is an integer from 1 to 10, e.g., 1 2, 3, 4, 5, 6, 7, 8, 9 or 10. For example, in some embodiments, the linker is GGGGS (SEQ ID NO: 114), GGGGSGGGGS (SEQ ID NO: 116), GGGGSGGGGSGGGGS (SEQ ID NO: 1), GGGGSGGGGSGGGGSGGGGS (SEQ ID NO: 117), or GGGGSGGGGSGGGGSGGGGSGGGGS (SEQ ID NO: 118).

Polyglycine linkers can suitably be used in the recombinant polypeptides of the disclosure. In some embodiments, a peptide linker comprises two consecutive glycines (2Gly), three consecutive glycines (3Gly), four consecutive glycines (4Gly) (SEQ ID NO: 119), five consecutive glycines (5Gly) (SEQ ID NO: 120), six consecutive glycines (6Gly) (SEQ ID NO: 121), seven consecutive glycines (7Gly) (SEQ ID NO: 122), eight consecutive glycines (8Gly) (SEQ ID NO: 123) or nine consecutive glycines (9Gly) (SEQ ID NO: 124).

Exemplary linker sequences are set forth in Table L below. An MBM of the disclosure may comprise one or more linkers of Table L.

TABLE L
Linker Sequences

| | Desi- | SEQ |
|---|---|---|
| Linker Sequence | gnation | ID NO: |
| (GGGGS)n | L1 | 114 |
| (GGGS)n | L2 | 125 |
| (GGS)n | L3 | N/A |
| (GS)n | L4 | N/A |
| (GSGGS)n | L5 | 126 |
| AAAGG | L6 | 127 |
| ADAAP | L7 | 128 |
| ADAAPTVSIFP | L8 | 129 |
| ADAAPTVSIFPP | L9 | 130 |
| AKTTAP | L10 | 131 |
| AKTTAPSVYPLAP | L11 | 132 |
| AKTTPKLEEGEFSEARV | L12 | 133 |
| AKTTPKLGG | L13 | 134 |
| AKTTPP | L14 | 135 |
| AKTTPPSVTPLAP | L15 | 136 |
| ASTKGP | L16 | 137 |
| ASTKGPSVFPLAPASTKGPSV | L17 | 138 |
| FPLAP | | |
| EGKSSGSGSESKST | L18 | 139 |
| GEGESGEGESGEGES | L19 | 140 |
| GEGESGEGESGEGESGEGES | L20 | 141 |
| GEGGSGEGGSGEGGS | L21 | 142 |
| GENKVEYAPALMALS | L22 | 143 |
| GGEGSGGEGSGGEGS | L23 | 144 |
| GGGESGGEGSGEGGS | L24 | 145 |
| GGGESGGGESGGGES | L25 | 146 |
| GGGGSGGGGS | L26 | 116 |
| GGGGSGGGGSGGGGS | L27 | 1 |
| GGGGSGGGGSGGGGSGGGGS | L28 | 117 |
| GGGKSGGGKSGGGKS | L29 | 147 |
| GGGKSGGKGSGKGGS | L30 | 148 |
| GGGS | L31 | 125 |
| GGGSG | L32 | 149 |
| GGKGSGGKGSGGKGS | L33 | 150 |
| GGS | L34 | N/A |
| GGSG | L35 | 151 |
| GGSGG | L36 | 152 |
| GGSGG | L37 | 152 |
| GGSGGGGSG | L38 | 153 |
| GGSGGGGGGGGS | L39 | 154 |
| GHEAAAVMQVQYPAS | L40 | 155 |
| GKGGSGKGGSGKGGS | L41 | 156 |
| GKGKSGKGKSGKGKS | L42 | 157 |
| GKGKSGKGKSGKGKSGKGKS | L43 | 158 |
| GKPGSGKPGSGKPGS | L44 | 159 |
| GKPGSGKPGSGKPGSGKPSGS | L45 | 160 |
| GPAKELTPLKEAKVS | L46 | 161 |
| GSAGSAAGSGEF | L47 | 162 |
| GSGGG | L48 | 163 |
| GSGSG | L49 | 164 |
| GSS | L50 | N/A |
| GSSG | L51 | 165 |
| GSSGGSGGSG | L52 | 166 |
| GSSGGSGGSGG | L53 | 167 |
| GSSGGSGGSGGS | L54 | 168 |
| GSSGGSGGSGGSG | L55 | 169 |
| GSSGGSGGSGGSGGGS | L56 | 170 |
| GSSGGSGGSGS | L57 | 171 |
| GSSGT | L58 | 172 |
| GSSSG | L59 | 173 |
| GSTSGSGKPGSGEGSTKG | L60 | 174 |
| GTAAAGAGAAGGAAAGAAG | L61 | 175 |
| GTSGSSGSGSGGSGSGGGG | L62 | 176 |
| IRPRAIGGSKPRVA | L63 | 177 |
| KESGSVSSEQLAQFRSLD | L64 | 178 |
| KTTPKLEEGEFSEAR | L65 | 179 |
| PRGASKSGSASQTGSAPGS | L66 | 180 |
| QPKAAP | L67 | 181 |
| QPKAAPSVTLFPP | L68 | 182 |
| RADAAAA(G4S)₄ | L69 | 183 |
| RADAAAAGGPGS | L70 | 184 |
| RADAAP | L71 | 185 |
| RADAAPTVS | L72 | 186 |
| SAKTTP | L73 | 187 |
| SAKTTPKLEEGEFSEARV | L74 | 188 |
| SAKTTPKLGG | L75 | 189 |
| STAGDTHLGGEDFD | L76 | 190 |
| TVAAP | L77 | 191 |
| TVAAPSVFIFPP | L78 | 192 |
| TVAAPSVFIFPPTVAAPSVFIFPP | L79 | 193 |

### 6.6. Dimerization Moieties

MBMs disclosed herein generally comprise one or more dimerization moieties. A dimerization moiety describes any polypeptide chain or amino acid sequence capable of facilitating an association between two polypeptide chains to form a dimer. In some embodiments, the dimerization moiety is an Fc domain.

### 6.6.1. Fc Regions

In certain aspects, the multispecific binding molecules of the disclosure comprise a pair of Fc domains that associate to form an Fc region. In native antibodies, Fc regions comprise hinge regions at their N-termini to form a constant domain. Throughout this disclosure, the reference to an Fc domain encompasses an Fc domain with a hinge domain at its N-terminus unless specified otherwise.

The Fc domains can be derived from any suitable species operably linked to an antigen-binding domain or component thereof. In one embodiment the Fc domain is derived from a human Fc domain. In preferred embodiments, an antigen-binding domain of a multispecific molecule of the disclosure is fused to an IgG Fc molecule. An antigen-binding domain may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific binding molecules, e.g., multispecific binding molecules described herein, the Fc domains might advantageously be different to allow for heterodimerization, as described in Section 6.6.1.2 below. In other embodiments, the two Fc domains of multispecific binding molecules disclosed herein are the same.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In multispecific binding molecules of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for multispecific binding molecules of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the multispecific binding molecules of the present disclosure do not comprise a tailpiece.

The Fc domains that are incorporated into the multispecific binding molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.6.1.1.

The Fc domains can also be altered to include modifications that improve manufacturability of asymmetric multispecific binding molecules, for example by allowing heterodimerization, which is the preferential pairing of non-identical Fc domains over identical Fc domains. Heterodimerization permits the production of multispecific binding molecules in which different polypeptide components are connected to one another by an Fc region containing Fc domains that differ in sequence. Examples of heterodimerization strategies are exemplified in Section 6.6.1.2.

It will be appreciated that any of the modifications mentioned above can be combined in any suitable manner to achieve the desired functional properties and/or combined with other modifications to alter the properties of the multispecific binding molecules.

TABLE F-1
Fc Sequences

| | | SEQ ID |
|---|---|---|
| Fc | Sequence | NO |
| hIgG1 Fc | EPKSCDKTHTCPPCPAPELLGGPSVFLFPPKPKDTLMISRTPEVTCVVVD | 20 |
| (amino acids | VSHEDPEVKFNWYVDGVEVHNAKTKPREEQYNSTYRVVSVLTVLHQDWLN | |
| 99-330 of | GKEYKCKVSNKALPAPIEKTISKAKGQPREPQVYTLPPSRDELTKNQVSL | |
| UniprotKB | TCLVKGFYPSDIAVEWESNGQPENNYKTTPPVLDSDGSFFLYSKLTVDKS | |
| P01857-1) | RWQQGNVFSCSVMHEALHNHYTQKSLSLSPGK | |
| hIgG2 Fc | ERKCCVECPPCPAPPVAGPSVFLFPPKPKDTLMISRTPEVTCVVVDVSHE | 21 |
| (amino acids | DPEVQFNWYVDGVEVHNAKTKPREEQFNSTFRVVSVLTVVHQDWLNGKEY | |
| 99-326 of | KCKVSNKGLPAPIEKTISKTKGQPREPQVYTLPPSREEMTKNQVSLTCLV | |
| UniprotKB | KGFYPSDISVEWESNGQPENNYKTTPPMLDSDGSFFLYSKLTVDKSRWQQ | |
| P01859-1) | GNVFSCSVMHEALHNHYTQKSLSLSPGK | |
| hIgG3 Fc | ELKTPLGDTTHTCPRCPEPKSCDTPPPCPRCPEPKSCDTPPPCPRCPEPK | 22 |
| (amino acids | SCDTPPPCPRCPAPELLGGPSVFLFPPKPKDTLMISRTPEVTCVVVDVSH | |
| 99-377 of | EDPEVQFKWYVDGVEVHNAKTKPREEQYNSTFRVVSVLTVLHQDWLNGKE | |
| UniprotKB | YKCKVSNKALPAPIEKTISKTKGQPREPQVYTLPPSREEMTKNQVSLTCL | |
| P01860-1) | VKGFYPSDIAVEWESSGQPENNYNTTPPMLDSDGSFFLYSKLTVDKSRWQ | |
| | QGNIFSCSVMHEALHNRFTQKSLSLSPGK | |
| hIgG4 Fc | ESKYGPPCPSCPAPEFLGGPSVFLFPPKPKDTLMISRTPEVTCVVVDVSQ | 23 |
| (amino acids | EDPEVQFNWYVDGVEVHNAKTKPREEQFNSTYRVVSVLTVLHQDWLNGKE | |
| 99-327 of | YKCKVSNKGLPSSIEKTISKAKGQPREPQVYTLPPSQEEMTKNQVSLTCL | |
| UniprotKB | VKGFYPSDIAVEWESNGQPENNYKTTPPVLDSDGSFFLYSRLTVDKSRWQ | |
| P01861-1) | EGNVFSCSVMHEALHNHYTQKSLSLSLGK | |
| hIgG4s Fc | ESKYGPPCPPCPAPPVAGPSVFLFPPKPKDTLMISRTPEVTCVVVDVSQE | 24 |
| | DPEVQFNWYVDGVEVHNAKTKPREEQFNSTYRVVSVLTVLHQDWLNGKEY | |
| | KCKVSNKGLPSSIEKTISKAKGQPREPQVYTLPPSQEEMTKNQVSLTCLV | |
| | KGFYPSDIAVEWESNGQPENNYKTTPPVLDSDGSFFLYSRLTVDKSRWQE | |
| | GNVFSCSVMHEALHNHYTQKSLSLSLGK | |
| hIgG1 PVA Fc | EPKSCDKTHTCPPCPAPPVAGPSVFLFPPKPKDTLMISRTPEVTCVVVDV | 25 |
| | SHEDPEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTVDKSR | |
| | WQQGNVFSCSVMHEALHNHYTQKSLSLSPGK | |
| hIgG1 PVA | EPKSCDKTHTCPPCPAPPVAGPSVFLFPPKPKDTLMISRTPEVTCVVVDV | 26 |
| star | SHEDPEVKFNWYVDGVEVHNAKTKPREEQYNSTYRVVSVLTVLHQDWING | |
| | KEYKCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTVDKSR | |
| | WQQGNVFSCSVMHEALHNRFTQKSLSLSPGK | |
| hIgG1s | DKKVEPKSCDKTHTCPPCPAPPVAGPSVFLFPPKPKDTLMISRTPEVTCV | 27 |
| | VVDVSQEDPEVQFNWYVDGVEVHNAKTKPREEQFNSTYRVVSVLTVLHQD | |
| | WLNGKEYKCKVSNKGLPSSIEKTISKAKGQPREPQVYTLPPSRDELTKNQ | |
| | VSLTCLVKGFYPSDIAVEWESNGQPENNYKTTPPVLDSDGSFFLYSKLTV | |
| | DKSRWQQGNVFSCSVMHEALHNHYTQKSLSLSPGK | |
| hIgG1 N180G, | EPKSCDKTHTCPPCPAPELLGGPSVFLFPPKPKDTLMISRTPEVTCVVVD | 28 |
| also referred to | VSHEDPEVKFNWYVDGVEVHNAKTKPREEQYGSTYRVVSVLTVLHQDWLN | |
| as N297G | GKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLSLSPGK | |
| hIgG2 variant | ERKCCVECPPCPAPPVAGPSVFLFPPKPKDTLMISRTPEVTCVVVDVSHE | 29 |
| | DPEVQFNWYVDGVEVHNAKTKPREEQFNSTFRVVSVLTVVHQDWLNGKEY | |
| | KCKVSNKGLPAPIEKTISKTKGQPREPQVYTLPPSREEMTKNQVSLTCLV | |
| | KGFYPSDIAVEWESNGQPENNYKTTPPMLDSDGSFFLYSKLTVDKSRWQQ | |
| | GNVFSCSVMHEALHNHYTQKSLSLSPGK | |
| hIgG4 S108P | ESKYGPPCPPCPAPEFLGGPSVFLFPPKPKDTLMISRTPEVTCVVVDVSQ | 30 |
| | EDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| IgG1PVA_hinge- | EPKSCDKTHTCPPCPAPPVAGPSVFLFPPKPKDTLMISRTPEVTCVVVDV | 31 |
| Fc knob | SHEDPEVKFNWYVDGVEVHNAKTKPREEQYNSTYRVVSVLTVLHQDWING | |
| | KEYKCKVSNKALPAPIEKTISKAKGQPREPQVYTLPPSRDELTKNQVSLW | |
| | CLVKGFYPSDIAVEWESNGQPENNYKTTPPVLDSDGSFFLYSKLTVDKSR | |
| | WQQGNVFSCSVMHEALHNHYTQKSLSLSPGK | |
| IgG1PVA_hinge- | EPKSCDKTHTCPPCPAPPVAGPSVFLFPPKPKDTLMISRTPEVTCVVVDV | 32 |
| Fc knob_star | SHEDPEVKFNWYVDGVEVHNAKTKPREEQYNSTYRVVSVLTVLHQDWING | |
| | KEYKCKVSNKALPAPIEKTISKAKGQPREPQVYTLPPSRDELTKNQVSLW | |
| | CLVKGFYPSDIAVEWESNGQPENNYKTTPPVLDSDGSFFLYSKLTVDKSR | |
| | WQQGNVFSCSVMHEALHNRFTQKSLSLSPGK | |
| IgG1PVA_hinge- | EPKSCDKTHTCPPCPAPPVAGPSVFLFPPKPKDTLMISRTPEVTCVVVDV | 33 |
| Fc knob_Cys | SHEDPEVKFNWYVDGVEVHNAKTKPREEQYNSTYRVVSVLTVLHQDWLNG | |
| | KEYKCKVSNKALPAPIEKTISKAKGQPREPQVYTLPPCRDELTKNQVSLW | |
| | CLVKGFYPSDIAVEWESNGQPENNYKTTPPVLDSDGSFFLYSKLTVDKSR | |
| | WQQGNVFSCSVMHEALHNHYTQKSLSLSPGK | |
| IgG1PVA_hinge- | EPKSCDKTHTCPPCPAPPVAGPSVFLFPPKPKDTLMISRTPEVTCVVVDV | 34 |
| Fc | SHEDPEVKFNWYVDGVEVHNAKTKPREEQYNSTYRVVSVLTVLHQDWLNG | |
| knob_Cys_star | KEYKCKVSNKALPAPIEKTISKAKGQPREPQVYTLPPCRDELTKNQVSLW | |
| | CLVKGFYPSDIAVEWESNGQPENNYKTTPPVLDSDGSFFLYSKLTVDKSR | |
| | WQQGNVFSCSVMHEALHNRFTQKSLSLSPGK | |
| IgG1PVA_hinge- | EPKSCDKTHTCPPCPAPPVAGPSVFLFPPKPKDTLMISRTPEVTCVVVDV | 35 |
| Fc Hole | SHEDPEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTVDKSR | |
| | WQQGNVFSCSVMHEALHNHYTQKSLSLSPGK | |
| IgG1PVA_hinge- | EPKSCDKTHTCPPCPAPPVAGPSVFLFPPKPKDTLMISRTPEVTCVVVDV | 36 |
| Fc Hole_star | SHEDPEVKFNWYVDGVEVHNAKTKPREEQYNSTYRVVSVLTVLHQDWING | |
| | KEYKCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTVDKSR | |
| | WQQGNVFSCSVMHEALHNRFTQKSLSLSPGK | |
| IgG1PVA_hinge- | EPKSCDKTHTCPPCPAPPVAGPSVFLFPPKPKDTLMISRTPEVTCVVVDV | 37 |
| Fc Hole_Cys | SHEDPEVKFNWYVDGVEVHNAKTKPREEQYNSTYRVVSVLTVLHQDWLNG | |
| | KEYKCKVSNKALPAPIEKTISKAKGQPREPQVCTLPPSRDELTKNQVSLS | |
| | CAVKGFYPSDIAVEWESNGQPENNYKTTPPVLDSDGSFFLVSKLTVDKSR | |
| | WQQGNVFSCSVMHEALHNHYTQKSLSLSPGK | |
| IgG1PVA_hinge- | EPKSCDKTHTCPPCPAPPVAGPSVFLFPPKPKDTLMISRTPEVTCVVVDV | 38 |
| Fc | SHEDPEVKFNWYVDGVEVHNAKTKPREEQYNSTYRVVSVLTVLHQDWLNG | |
| Hole_Cys_star | KEYKCKVSNKALPAPIEKTISKAKGQPREPQVCTLPPSRDELTKNQVSLS | |
| | CAVKGFYPSDIAVEWESNGQPENNYKTTPPVLDSDGSFFLVSKLTVDKSR | |
| | WQQGNVFSCSVMHEALHNRFTQKSLSLSPGK | |

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to any one of the sequences disclosed in Table F-1.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:20. In cases where an Fc domain comprises at least 90% sequence identity and less than 100% sequence identity to SEQ ID NO:20 (e.g., between 90% and 99% sequence identity to SEQ ID NO:20), an Fc domain may also comprise one or more amino acid substitutions described herein, for example one or more substitutions that reduce effector function (e.g., as described in Section 6.6.1.1) and/or one or more substitutions that promote Fc heterodimerization (e.g., as described in Section 6.6.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:21. In cases where an Fc domain comprises at least 90% sequence identity and less than 100% sequence identity to SEQ ID NO:21 (e.g., between 90% and 99% sequence identity to SEQ ID NO:21), an Fc domain may also comprise one or more amino acid substitutions described herein, for example one or more substitutions that reduce effector function (e.g., as described in Section 6.6.1.1) and/or one or more substitutions that promote Fc heterodimerization (e.g., as described in Section 6.6.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:22. In cases where an Fc domain comprises at least 90% sequence identity and less than 100% sequence identity to SEQ ID NO:22 (e.g., between 90% and 99% sequence identity to SEQ ID NO:22), an Fc domain may also comprise one or more amino acid substitutions described herein, for example one or more substitutions that reduce effector function (e.g., as described in Section 6.6.1.1) and/or one or more substitutions that promote Fc heterodimerization (e.g., as described in Section 6.6.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:23. In cases where an Fc domain comprises at least 90% sequence identity and less than 100% sequence identity to SEQ ID NO:23 (e.g., between 90% and 99% sequence identity to SEQ ID NO:23), an Fc domain may also comprise one or more amino acid substitutions described herein, for example one or more substitutions that reduce effector function (e.g., as described in Section 6.6.1.1) and/or one or more substitutions that promote Fc heterodimerization (e.g., as described in Section 6.6.1.2).

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:24.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:25.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:26.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:27.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:28.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:29.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:30.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:31.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:32.

In some aspects, an Fc domain comprises an amino acid sequence having at least about 90%, at least about 91%, at least about 92%, at least about 93%, at least about 94% at least about 95%, at least about 96%, at least about 97%, at least about 98%, at least about 99%, or 100% sequence identity to SEQ ID NO:33.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:34.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:35.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:36.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:37.

In some aspects, an Fc domain comprises an amino acid sequence having at least about 90%, at least about 91%, at least about 92%, at least about 93%, at least about 94%, at least about 95%, at least about 96%, at least about 97%, at least about 98%, at least about 99%, or 100% sequence identity to SEQ ID NO:38.

6.6.1.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of a multispecific binding molecule) or the Fc region (e.g., one or both Fc domains of a multispecific binding molecule that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an IgG Fc domain or region, particularly a human IgG Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table F-2 below: In some embodiments, the Fc domain includes only the bolded portion of the sequences shown below:

TABLE F-2

| | | SEQ ID |
|---|---|---|
| Fc Domain | Sequence | NO |
| SEQ ID NO: 1 of | DKRVESKYGP PCPPCPAPPV AGPSVFLFPP KPKDTLMISR | 194 |
| WO2014/121087 | TPEVTCVVVD VSQEDPEVQF NWYVDGVEVH NAKTKPREEQ | |
| | FNSTYRVVSV LTVLHQDWLN GKEYKCKVSN KGLPSSIEKT | |
| | ISKAKGQPRE PQVYTLPPSQ EEMTKNQVSL TCLVKGFYPS | |
| | DIAVEWESNG QPENNYKTTP PVLDSDGSFF LYSRLTVDKS | |
| | RWQEGNVFSC SVMHEALHNH YTQKSLSLSL GK | |
| SEQ ID NO: 2 of | DKKVEPKSCD KTHTCPPCPA PPVAGPSVFL FPPKPKDTLM | 27 |
| WO2014/121087 | ISRTPEVTCV VVDVSQEDPE VQFNWYVDGV EVHNAKTKPR | |
| | EEQFNSTYRV VSVLTVLHQD WLNGKEYKCK VSNKGLPSSI | |
| | EKTISKAKGQ PREPQVYTLP PSRDELTKNQ VSLTCLVKGF | |
| | YPSDIAVEWE SNGQPENNYK TTPPVLDSDG SFFLYSKLTV | |
| | DKSRWQQGNV FSCSVMHEAL HNHYTQKSLS LSPGK | |
| SEQ ID NO: 30 | ASTKGPSVFP LAPSSKSTSG GTAALGCLVK DYFPEPVTVS | 195 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| SEQ ID NO: 31 | ASTKGPSVFP LAPCSRSTSE STAALGCLVK DYFPEPVTVS | 196 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNHYTQKSL | |
| | SLSLGK | |
| SEQ ID NO: 37 | ASTKGPSVFP LAPSSKSTSG GTAALGCLVK DYFPEPVTVS | 197 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNRFTQ | |
| | KSLSLSPGK | |
| SEQ ID NO: 38 | ASTKGPSVFP LAPCSRSTSE STAALGCLVK DYFPEPVTVS | 198 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNRFTQKSL | |
| | SLSLGK | |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof). 6.6.1.2. Fc Heterodimerization Variants

Certain multispecific binding molecules entail dimerization between two Fc domains that, unlike a native immunoglobulin, are operably linked to non-identical N-terminal or C-terminal regions. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the multispecific binding molecules of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides multispecific binding molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine I, phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the multispecific binding molecules comprise a first CH3 domain and a second Ig CH3 domain, wherein the first and second Ig CH3 domains differ from one another by at least one amino acid, and wherein at least one amino acid difference reduces binding of the multispecific binding molecules to Protein A as compared to a corresponding multispecific binding molecule lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.6.1.3. Hinge Domains

The multispecific binding molecules of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric multispecific binding molecule formed by the association of two Fc domains) can comprise two associated hinge sequences on separate polypeptide chains. Sometimes, the two associated hinge sequences are referred to as a “hinge region”. In certain embodiments of multispecific binding molecules of the disclosure, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, a multispecific binding molecule of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the multispecific binding molecules of the disclosure comprise a modified hinge region that reduces binding affinity for an Fcγ receptor relative to a wild-type hinge region of the same isotype (e.g., human IgG1 or human IgG4).

In one embodiment, the multispecific binding molecules of the disclosure comprise an Fc region in which each Fc domain possesses an intact hinge domain at its N-terminus, where each Fc domain and hinge domain is derived from IgG4 and each hinge domain comprises the modified sequence CPPC (SEQ ID NO:199). The core hinge region of human IgG4 contains the sequence CPSC (SEQ ID NO:200) compared to IgG1 that contains the sequence CPPC (SEQ ID NO:199). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.6.1.3.1. Chimeric Hinge Sequences

The hinge domain can be a chimeric hinge domain (also “chimeric hinge region”).

For example, a chimeric hinge domain may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO:201) (previously disclosed as SEQ ID NO:8 of WO2014/121087, which is incorporated by reference in its entirety herein) or ESKYGPPCPPCPAPPVA (SEQ ID NO:202) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1.1).

6.6.1.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG-(233-236), G---(233-236) and no G(233-236). Optionally, the hinge domain amino acid sequence comprises CPPCPAPGGG-GPSVF (SEQ ID NO: 203) (previously disclosed as SEQ ID NO:1 of WO2016161010A2), CPPCPAPGG-GPSVF (SEQ ID NO: 204) (previously disclosed as SEQ ID NO:2 of WO2016161010A2), CPPCPAPG---GPSVF (SEQ ID NO: 205) (previously disclosed as SEQ ID NO:3 of WO2016161010A2), or CPPCPAP----GPSVF (SEQ ID NO: 206) (previously disclosed as SEQ ID NO:4 of WO2016161010A2).

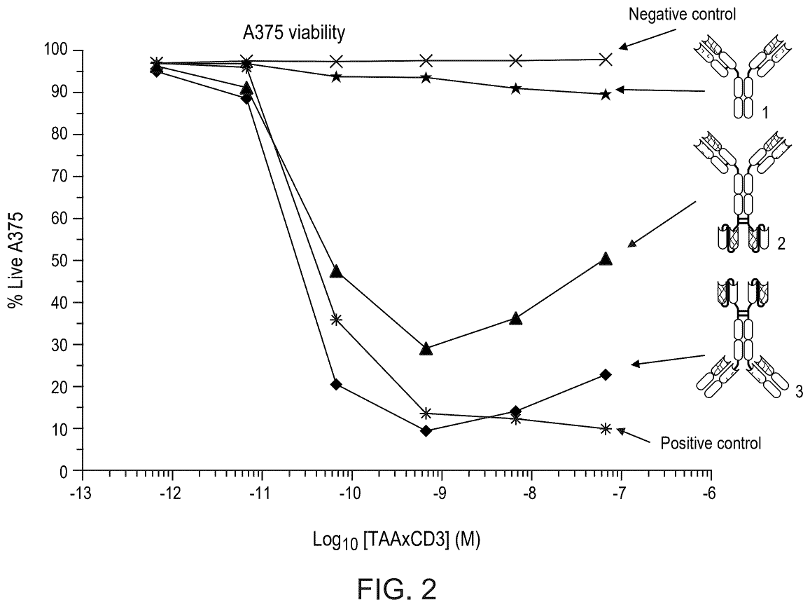

The modified hinge regions described above can be incorporated into a heavy chain constant region, which typically includes CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1.1).

### 6.7. Nucleic Acids and Host Cells

In another aspect, the disclosure provides nucleic acids encoding the multispecific binding molecules of the disclosure. In some embodiments, the multispecific binding molecules are encoded by a single nucleic acid. In other embodiments, for example in the case of a heterodimeric molecule or a molecule comprising a component composed of more than one polypeptide chain, the multispecific binding molecules can be encoded by a plurality (e.g., two, three, four or more) nucleic acids.

A single nucleic acid can encode a multispecific binding molecule that comprises a single polypeptide chain, a multispecific binding molecule that comprises two or more polypeptide chains, or a portion of a multispecific binding molecule that comprises more than two polypeptide chains (for example, a single nucleic acid can encode two polypeptide chains of a multispecific binding molecule comprising three, four or more polypeptide chains, or three polypeptide chains of a multispecific binding molecule comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a multispecific binding molecule comprising two or more polypeptide chains is encoded by two or more nucleic acids. The number of nucleic acids encoding a multispecific binding molecule can be equal to or less than the number of polypeptide chains in the multispecific binding molecule (for example, when more than one polypeptide chains are encoded by a single nucleic acid).

The nucleic acids of the disclosure can be DNA (e.g., plasmid) or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.8. Pharmaceutical Compositions

The multispecific binding molecules MBM of the disclosure may be in the form of a composition comprising the MBM and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the multispecific binding molecule and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of a multispecific binding molecule of the disclosure per dose. The quantity of MBM included in a unit dose will depend on the disease being treated, as well as other factors as are well known in the art. Such unit dosages may be in the form of a lyophilized dry powder containing an amount of multispecific binding molecule suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of multispecific binding molecule suitable for a single administration.

The pharmaceutical compositions may also be supplied in bulk from containing quantities of MBMs suitable for multiple administrations.

Pharmaceutical compositions may be prepared for storage as lyophilized formulations or aqueous solutions by mixing a multispecific binding molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16^{th} edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalkonium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of multispecific binding molecule.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.9. Methods of Use

The MBMs of the disclosure are useful in eliciting immune responses against cancer and thus treating cancer in a subject. The MBMs of the disclosure may be administered per se or in any suitable pharmaceutical composition, including a composition as described in Section 6.8.

In one aspect, MBMs of the disclosure for use as a medicament are provided. In further aspects, MBMs of the disclosure for use in treating a disease are provided. In certain embodiments, MBMs of the disclosure for use in a method of treatment are provided. In one embodiment, the disclosure provides an MBM as described herein for use in the treatment of a disease in a subject in need thereof. In certain embodiments, the disclosure provides an MBM for use in a method of treating a subject having a disease comprising administering to the individual a therapeutically effective amount of the MBM. In certain embodiments the disease to be treated is a proliferative disorder. In a preferred embodiment the disease is cancer.

In further embodiments, the disclosure provides an MBM for use in stimulating the immune system, particularly against a tumor cell. In certain embodiments, the disclosure provides an MBM for use in a method of stimulating the immune system in a subject comprising administering to the individual an effective amount of the MBM to stimulate the immune system. An “individual” according to any of the above embodiments is a mammal, preferably a human. “Stimulation of the immune system” according to any of the above embodiments may include any one or more of a general increase in immune function, an increase in T cell function, and/or an increase in T cell proliferation.

In a further aspect, the disclosure provides for the use of an MBM of the disclosure in the manufacture or preparation of a medicament for the treatment or prevention of a disease in a subject in need thereof. In one embodiment, the medicament is for use in a method of treating a disease comprising administering to a subject having the disease a therapeutically effective amount of the medicament.

In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments is a mammal, preferably a human. “Stimulation of the immune system” according to any of the above embodiments may include any one or more of a general increase in immune function, an increase in T cell function, and/or an increase in T cell proliferation.

In a further aspect, the disclosure provides a method for treating a disease in a subject, comprising administering to said individual a therapeutically effective amount of an MBM of the disclosure. In one embodiment a composition is administered to said individual, comprising the MBM of the disclosure in a pharmaceutically acceptable form, e.g., as described in Section 6.8. In certain embodiments the disease to be treated is a proliferative disorder. In a preferred embodiment the disease is cancer. In a further aspect, the disclosure provides a method for stimulating the immune system in a subject, comprising administering to the individual an effective amount of an MBM (e.g., in the form of a pharmaceutical composition, e.g., as described in Section 6.8) to stimulate the immune system. An “individual” according to any of the above embodiments is a mammal, preferably a human.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using an MBM of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the MBMs, when used in the context of therapeutic treatment, may not provide a cure but may only provide partial benefit. In some embodiments, a physiological change having some benefit is also considered therapeutically beneficial. Thus, in some embodiments, an amount of MBM that provides a physiological change is considered an “effective amount” or a “therapeutically effective amount”. The subject, patient, or individual in need of treatment is typically a mammal, more specifically a human.

The appropriate dosage of an MBM of the disclosure (when used alone or in combination with one or more other additional therapeutic agents) will depend on the type of disease to be treated, the route of administration, the body weight of the patient, the particular MBM, the severity and course of the disease, previous or concurrent therapeutic interventions, the patient's clinical history and response to the MBM (and any adjunct therapies), and the discretion of the attending physician. The practitioner responsible for administration will, in any event, determine the concentration of active ingredient(s) in a composition(s) and appropriate dose(s) for the individual subject. Various dosing schedules including but not limited to single or multiple administrations over various time-points.

The MBMs of the disclosure will generally be used in an amount effective to achieve the intended purpose. For use to treat proliferative condition, the MBMs of the disclosure, or pharmaceutical compositions thereof, are administered or applied in a therapeutically effective amount. Determination of a therapeutically effective amount is well within the capabilities of those skilled in the art, especially in light of the detailed disclosure provided herein. For clarity, a therapeutically effective amount is an amount that can elicit a beneficial physiological change (whether in a patient population or in a specific patient), and not necessarily an amount that will lead to a cure or remission of a proliferative disorder.

### 6.9.1. Combination Therapy

The therapeutic methods described above encompass combination therapy methods, whereby the MBMs of the disclosure are administered in combination with one or more other agents in therapy.

For instance, an MBM of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In further embodiments, the additional therapeutic agent is an immunotherapeutic agent, for example a checkpoint inhibitor (e.g., anti-PD1 antibody, anti-PDL1 antibody, or the like).

Such other agents are suitably present in combination in amounts that are effective for the purpose intended. The effective amount of such other agents depends on the amount of MBM used, the type of disorder or treatment, and other factors discussed above.

In some embodiments, the MBMs of the disclosure stimulate T cells against tumor cells through stimulation of the T-cell receptor (TCR)-CD3 complex. The TCR-CD3 complex is composed of a diverse ap TCR heterodimer noncovalently associated with the invariant CD3 dimers CD3εγ, CD3εδ, and CD3ζζ. The TCR mediates recognition of antigenic peptides bound to MHC molecules (pMHC; also described herein as “HLA-bound peptide”), whereas the CD3 molecules transduce activation signals to the T cell. See Mariuzza et al., 2020, J Biol Chem. 295(4): 914-925. Thus, an MBM whose antigen targeting moiety binds to a pMHC present on the tumor cell and the TCA binds to a TCR-CD3 complex component such as CD3, the MBM can stimulate the T cell against the tumor cell.

This T cell stimulation elicited by an MBM of the disclosed can be boosted by use of a second therapeutic agent that promotes signaling via a costimulatory pathway such as CD28, ICOS or CTLA4. See, e.g., Rudd & Schneider, 2003, Nature Reviews Immunology 3:544-556. In some embodiments, the second therapeutic agent is an anti-CD28, anti-ICOS or anti-CTLA4 antibody. Advantageously, a second therapeutic agent that promotes signaling via a costimulatory pathway is a multispecific antibody that comprises not only a costimulatory receptor targeting moiety but a second targeting moiety that binds to a tumor antigen, e.g., a tumor antigen bound present on the same tumor cell that is bound by the antigen targeting moiety in the MBM. The tumor antigen bound by the multispecific antibody may be different from the antigen bound by the targeting moiety in the MBM.

The combination will generally be used in an amount effective to treat a proliferative condition. Determination of a therapeutically effective amount is well within the capabilities of those skilled in the art, especially in light of the detailed disclosure provided herein. For clarity, a therapeutically effective amount of the combination is an amount of the combination that can elicit a beneficial physiological change (e.g., in a patient population or in a specific patient), and not necessarily an amount that will lead to a cure or remission of a proliferative disorder. Further, the amounts of each component used in the combination may not be therapeutically effective amounts of the individual components, provided the combination as a whole is a therapeutically effective amount.

Such combination therapies noted above encompass combined administration (where two or more therapeutic agents are included in the same compositions or separate composition administered in the same treatment session), and separate administration, in which case, administration of the MBM of the disclosure can occur prior to, simultaneously, and/or following, administration of the additional therapeutic agent.

MBMs and combination therapies of the disclosure can also be used in combination with radiation therapy and/or immunotherapy. In particular embodiments, combination therapies of the disclosure include combination of an MBM and an immunotherapeutic agent such as a checkpoint inhibitor. In some embodiments, the immunotherapeutic agent is an anti-PD1 antibody. In some embodiments, the immunotherapeutic agent is an anti-PDL1 antibody.

### 7. Specific Embodiments

While various specific embodiments have been illustrated and described, it will be appreciated that various changes can be made without departing from the spirit and scope of the disclosure(s). The present disclosure is exemplified by the numbered embodiments set forth below.
1. A multispecific binding molecule (MBM) comprising:
   (a) a first polypeptide chain comprising, in an N- to C-terminal orientation (i) a first antigen binding domain; (ii) an optional first linker; (iii) a first dimerization moiety; (iv) an optional second linker; and (v) a first portion of a first Fab;
   (b) a second polypeptide chain comprising, in an N- to C-terminal orientation (i) a second antigen binding domain; (ii) an optional third linker; (iii) a second dimerization moiety; (iv) an optional fourth linker; and (v) a first portion of a second Fab;
   (c) a third polypeptide chain comprising a second portion of the first Fab associated with the first portion of the first Fab; and
   (d) a fourth polypeptide chain comprising a second portion of the second Fab associated with the first portion of the second Fab.
2. The MBM of embodiment 1, wherein the first and second dimerization moieties are Fc domains.
3. The MBM of embodiment 2, wherein the Fc domains are IgG1 Fc domains.
4. The MBM of embodiment 2, wherein the Fc domains are IgG4 Fc domains.
5. The MBM of embodiment 2, wherein the Fc domains are each an Fc domain set forth in Table F-1.
6. The MBM of embodiment 2, wherein the Fc domains each comprise an amino acid sequence having at least 90% sequence identity to any one of SEQ ID NOs:20 to 38, optionally comprising one or more mutations to reduce effector function, e.g., as described in Section 6.6.1.1.
7. The MBM of embodiment 2, wherein the Fc domains each comprise an amino acid sequence having at least 95% sequence identity to any one of SEQ ID NOs: 20 to 38, optionally comprising one or more mutations to reduce effector function, e.g., as described in Section 6.6.1.1.
8. The MBM of embodiment 2, wherein the Fc domains each comprise an amino acid sequence having at least 98% sequence identity to any one of SEQ ID NOs: 20 to 38, optionally comprising one or more mutations to reduce effector function, e.g., as described in Section 6.6.1.1.
9. The MBM of embodiment 2, wherein the Fc domains each comprise an amino acid sequence having at least 99% sequence identity to any one of SEQ ID NOs: 20 to 38, optionally comprising one or more mutations to reduce effector function, e.g., as described in Section 6.6.1.1.
10. The MBM of embodiment 2, wherein the Fc domains each comprise the amino acid sequence of any one of SEQ ID NOs: 20 to 38, optionally comprising one or more mutations to reduce effector function, e.g., as described in Section 6.6.1.1.
11. The MBM of embodiment 2, wherein the Fc domains each comprise an amino acid sequence having at least 90% sequence identity to SEQ ID NO:20, optionally comprising one or more mutations to reduce effector function, e.g., as described in Section 6.6.1.1.
12. The MBM of embodiment 2, wherein the Fc domains each comprise an amino acid sequence having at least 95% sequence identity to SEQ ID NO:20, optionally comprising one or more mutations to reduce effector function, e.g., as described in Section 6.6.1.1.
13. The MBM of embodiment 2, wherein the Fc domains each comprise an amino acid sequence having at least 98% sequence identity to SEQ ID NO:20, optionally comprising one or more mutations to reduce effector function, e.g., as described in Section 6.6.1.1.
14. The MBM of embodiment 2, wherein the Fc domains each comprise an amino acid sequence having at least 90% sequence identity to SEQ ID NO:21, optionally comprising one or more mutations to reduce effector function, e.g., as described in Section 6.6.1.1.
15. The MBM of embodiment 2, wherein the Fc domains each comprise an amino acid sequence having at least 95% sequence identity to SEQ ID NO:21, optionally comprising one or more mutations to reduce effector function, e.g., as described in Section 6.6.1.1.
16. The MBM of embodiment 2, wherein the Fc domains each comprise an amino acid sequence having at least 98% sequence identity to SEQ ID NO:21, optionally comprising one or more mutations to reduce effector function, e.g., as described in Section 6.6.1.1.
17. The MBM of embodiment 2, wherein the Fc domains each comprise an amino acid sequence having at least 90% sequence identity to SEQ ID NO:22, optionally comprising one or more mutations to reduce effector function, e.g., as described in Section 6.6.1.1.
18. The MBM of embodiment 2, wherein the Fc domains each comprise an amino acid sequence having at least 95% sequence identity to SEQ ID NO:22, optionally comprising one or more mutations to reduce effector function, e.g., as described in Section 6.6.1.1.
19. The MBM of embodiment 2, wherein the Fc domains each comprise an amino acid sequence having at least 98% sequence identity to SEQ ID NO:22, optionally comprising one or more mutations to reduce effector function, e.g., as described in Section 6.6.1.1.
20. The MBM of embodiment 2, wherein the Fc domains each comprise an amino acid sequence having at least 90% sequence identity to SEQ ID NO:23, optionally comprising one or more mutations to reduce effector function, e.g., as described in Section 6.6.1.1.
21. The MBM of embodiment 2, wherein the Fc domains each comprise an amino acid sequence having at least 95% sequence identity to SEQ ID NO:23, optionally comprising one or more mutations to reduce effector function, e.g., as described in Section 6.6.1.1.
22. The MBM of embodiment 2, wherein the Fc domains each comprise an amino acid sequence having at least 98% sequence identity to SEQ ID NO:23, optionally comprising one or more mutations to reduce effector function, e.g., as described in Section 6.6.1.1.
23. The MBM of any one of embodiments 2 to 22, wherein the Fc domains are associated to form an Fc region.
24. The MBM of embodiment 23, wherein the Fc region is an Fc homodimer.
25. The MBM of embodiment 23, wherein the Fc region is an Fc heterodimer (e.g., with one Fc domain comprising knob mutations, the other Fc domain comprising hole mutations and optionally one of the domains comprising star mutations, e.g. as described in Section 6.6.1.2).
26. The MBM of any one of embodiments 1 to 25, wherein the first linker, if present, is a glycine-serine linker.
27. The MBM of any one of embodiments 1 to 26, wherein the first linker, if present, is a linker set forth in Table L.
28. The MBM of any one of embodiments 1 to 27, wherein the second linker, if present, is a glycine-serine linker.
29. The MBM of any one of embodiments 1 to 27, wherein the second linker, if present, is a linker set forth in Table L.
30. The MBM of any one of embodiments 1 to 29, wherein the third linker, if present, is a glycine-serine linker.
31. The MBM of any one of embodiments 1 to 29, wherein the third linker, if present, is a linker set forth in Table L.
32. The MBM of any one of embodiments 1 to 31, wherein the fourth linker, if present, is a glycine-serine linker.
33. The MBM of any one of embodiments 1 to 31, wherein the fourth linker, if present, is a linker set forth in Table L.
34. The MBM of any one of embodiments 1 to 33, wherein one, two, three, or all four of the first, second, third, and fourth linkers are identical.
35. The MBM of any one of embodiments 1 to 34, wherein the first portion of the first Fab comprises a VH domain.
36. The MBM of embodiment 35, wherein the second portion of the first Fab comprises a VL domain.
37. The MBM of any one of embodiments 1 to 34, wherein the first portion of the first Fab comprises a VL domain.
38. The MBM of embodiment 37, wherein the second portion of the first Fab comprises a VH domain.
39. The MBM of any one of embodiments 35 to 38, wherein the first portion of the first Fab further comprises a CH1 domain.
40. The MBM of embodiment 39, wherein the second portion of the first Fab further comprises a CL domain.
41. The MBM of any one of embodiments 35 to 38, wherein the first portion of the first Fab further comprises a CL domain.
42. The MBM of embodiment 41, wherein the second portion of the first Fab further comprises a CH1 domain.
43. The MBM of any one of embodiments 1 to 42, wherein the first portion of the second Fab comprises a VH domain.
44. The MBM of embodiment 43, wherein the second portion of the second Fab comprises a VL domain.
45. The MBM of any one of embodiments 1 to 42, wherein the first portion of the second Fab comprises a VL domain.
46. The MBM of embodiment 45, wherein the second portion of the second Fab comprises a VH domain.
47. The MBM of any one of embodiments 43 to 46, wherein the first portion of the second Fab further comprises a CH1 domain.
48. The MBM of embodiment 47, wherein the second portion of the second Fab further comprises a CL domain.
49. The MBM of any one of embodiments 43 to 46, wherein the first portion of the second Fab further comprises a CL domain.
50. The MBM of embodiment 49, wherein the second portion of the second Fab further comprises a CH1 domain.
51. The MBM of any one of embodiments 1 to 50, wherein the first Fab and the second Fab each comprise a universal light chain.
52. The MBM of any one of embodiments 1 to 51, wherein the first antigen binding domain is a T cell antigen (TCA) targeting moiety.
53. The MBM of embodiment 52, wherein the first antigen binding domain is a TCR targeting moiety.
54. The MBM of embodiment 53, wherein the first antigen binding domain is a CD3 targeting moiety.
55. The MBM of embodiment 54, wherein the first antigen binding domain specifically binds to human CD3.
56. The MBM of embodiment 53, wherein the first antigen binding domain is a TCRαβ targeting moiety.
57. The MBM of embodiment 56, wherein the first antigen binding domain specifically binds to human TCRαβ.
58. The MBM of embodiment 53, wherein the first antigen binding domain is a TCRγδ targeting moiety.
59. The MBM of embodiment 52, wherein the first antigen binding domain (a) comprises the (i) CDR or (ii) VH and VL sequences of an antibody set forth in Table T-1 or (b) competes with the antibody set forth in Table T-1 for binding to its target.
60. The MBM of embodiment 52, wherein the first antigen binding domain (a) comprises the CDR sequences of an antibody set forth in Table T-2 or (b) competes with the antibody set forth in Table T-2 for binding to its target.
61. The MBM of any one of embodiments 1 to 51, wherein the first antigen binding domain comprises means for binding a T cell antigen.
62. The MBM of embodiment 61, wherein the first antigen binding domain comprises means for binding human CD3.
63. The MBM of any one of embodiments 1 to 62, wherein the first antigen binding domain is an scFv.
64. The MBM of embodiment 63, wherein the scFv (of the first antigen binding domain) has the configuration, in N- to C-terminal orientation, VL-linker-VH.
65. The MBM of embodiment 63, wherein the scFv (of the first antigen binding domain) has the configuration, in N- to C-terminal orientation, VH-linker-VL.
66. The MBM of any one of embodiments 63 to 65, wherein the VH of the scFv (of the first antigen binding domain) comprises identical CDRs to the VH of the first Fab.
67. The MBM of any one of embodiments 63 to 65, wherein the VL of the scFv (of the first antigen binding domain) comprises identical CDRs to the VL of the first Fab.
68. The MBM of any one of embodiments 1 to 62, wherein the first antigen binding domain is an sdVH.
69. The MBM of any one of embodiments 1 to 62, wherein the first antigen binding domain is a VHH.
70. The MBM of any one of embodiments 1 to 69, wherein the first Fab is a TCA targeting moiety.
71. The MBM of embodiment 70, wherein the first Fab is a TCR targeting moiety.
72. The MBM of embodiment 71, wherein the first Fab is a CD3 targeting moiety.
73. The MBM of embodiment 72, wherein the first Fab specifically binds to human CD3.
74. The MBM of embodiment 71, wherein the first Fab is a TCRαβ targeting moiety.
75. The MBM of embodiment 74, wherein the first Fab specifically binds to human TCRαβ.
76. The MBM of embodiment 74, wherein the first Fab is a TCRγδ targeting moiety.
77. The MBM of embodiment 70, wherein the first Fab (a) comprises the (i) CDR or (ii) VH and VL sequences of an antibody set forth in Table T-1 or (b) competes with the antibody set forth in Table T-1 for binding to its target.
78. The MBM of embodiment 70, wherein the first Fab (a) comprises the CDR sequences of an antibody set forth in Table T-2 or (b) competes with the antibody set forth in Table T-2 for binding to its target.
79. The MBM of any one of embodiments 1 to 69, wherein the first Fab comprises means for binding a T cell antigen.
80. The MBM of embodiment 79, wherein the first Fab comprises means for binding human CD3.
81. The MBM of any one of embodiments 1 to 80, wherein the first antigen binding domain and the first Fab specifically bind to the same target.
82. The MBM of any one of embodiments 1 to 80, wherein the first antigen binding domain and the first Fab specifically bind to different targets.
83. The MBM of any one of embodiments 1 to 82, wherein the second antigen binding domain is an antigen targeting moiety.
84. The MBM of embodiment 83, wherein the second antigen binding domain is a tumor antigen targeting moiety.
85. The MBM of embodiment 84, wherein the second antigen binding domain specifically binds to a tumor antigen set forth in Table T-3.
86. The MBM of embodiment 84, wherein the second antigen binding domain specifically binds to a tumor antigen set forth in Table P-1.
87. The MBM of embodiment 84, wherein the second antigen binding domain specifically binds to a tumor antigen set forth in Table P-2.
88. The MBM of any one of embodiments 84 to 87, wherein the second antigen binding domain (a) comprises the (i) CDR or (ii) VH and VL sequences of an antibody or TCR set forth in Table T-3 or (b) competes with an antibody or TCR set forth in Table T-3 for binding to its target.
89. The MBM of any one of embodiments 84 to 87, wherein the second antigen binding domain (a) comprises the CDR sequences of an antibody set forth in Table T-4 or (b) competes with an antibody set forth in Table T-4 for binding to its target.
90. The MBM of any one of embodiments 83 to 89, wherein the second antigen binding domain is a low-density antigen targeting moiety.
91. The MBM of embodiment 90, wherein the low-density antigen is an antigen expressed at no more than 5000 copies per cell.
92. The MBM of embodiment 90, wherein the low-density antigen is an antigen expressed at no more than 4000 copies per cell.
93. The MBM of embodiment 90, wherein the low-density antigen is an antigen expressed at no more than 3000 copies per cell.
94. The MBM of embodiment 90, wherein the low-density antigen is an antigen expressed at no more than 2000 copies per cell.
95. The MBM of embodiment 90, wherein the low-density antigen is an antigen expressed at no more than 1000 copies per cell.
96. The MBM of embodiment 90, wherein the low-density antigen is an antigen expressed at no more than 500 copies per cell.
97. The MBM of embodiment 90, wherein the low-density antigen is an antigen expressed at no more than 250 copies per cell.
98. The MBM of embodiment 90, wherein the low-density antigen is an antigen expressed at no more than 100 copies per cell.
99. The MBM of embodiment 90, wherein the low-density antigen is an antigen expressed at no more than 50 copies per cell.
100. The MBM of embodiment 90, wherein the low-density antigen is an antigen expressed at no more than 40 copies per cell.
101. The MBM of embodiment 90, wherein the low-density antigen is an antigen expressed at no more than 30 copies per cell.
102. The MBM of embodiment 90, wherein the low-density antigen is an antigen expressed at no more than 20 copies per cell.
103. The MBM of embodiment 90, wherein the low-density antigen is an antigen expressed at no more than 10 copies per cell.
104. The MBM of any one of embodiments 83 to 103, wherein the second antigen binding domain is an HLA-bound peptide antigen targeting moiety.
105. The MBM of embodiment 104, wherein the HLA-bound peptide is a viral peptide.
106. The MBM of embodiment 105, wherein the viral peptide is HPV16 E7 (11-20), HPV E716 (11-19), HPV16 E7 (82-90), CMV pp65 (495-503), HIV P17 (77-85), HIV RT (896-904), HIV GAG (41-49), or ENV (183-191).
107. The MBM of embodiment 105, wherein the viral peptide is a peptide set forth in Table P-1.
108. The MBM of any one of embodiments 104 to 107, wherein the HLA-bound peptide is a tumor-associated peptide.
109. The MBM of embodiment 108, wherein the tumor-associated peptide is LCMV derived peptide gp33-41, ACP3 (PAP) (112-120), AFP (58-68), AFP (173-181), APF (126-134), BALF(276-284), CEA (571-579), CMV pp65 (495-503), DNTT (475-483), DNTT (250-258), EBNA-1 (562-570), FLU-M1 (58-66), gp100 (154-162), gp100 (209-217), HBV Core (18-27), Her2/neu (369-377; V2v9); HPV16 E7 (11-20), HPV E716 (11-19), HPV16 E7 (82-90), IGF2BP3 (552-560), KLK4 (11-19), KLK4 (215-223), KLK4 (105-113), KRAS (6-14; G12V), KRAS (4-12; K5L/G12V), KRAS (5-14; G12V), KRAS (5-14; G12D), KRAS (10-18; G12D), KRAS (10-19; G12D), KRAS (8-16; G12D), KRAS (8-16; G12D), KRAS (7-16; G12D), KRAS (7-16; G12D), LMP1 (125-133), LMP2 (426-434), MAGE-A3 (112-120), MAGE-A3 (168-176), MAGE-A3 (271-279), MAGE-A3 (108-116), MAGE-A4 (230-239), MAGE-A4 (286-294), MAGE-A10 (137-145), MAGE-A10 (254-262), MAGE-A10 (310-318), MPO (132-140), MPO (549-557), MPO (571-579), NYESO1 (157-165, C165A), NYESO1 (157-165, C165V), p54 WT (264-272), PAP-3 (136-143), PRAME (425-433), PRAME (312-320), PSA (159-169), PSA (241-249), PSA (79-89), PSA (170-178), PSA (82-90), PSMA (4-12), PSMA (135-145), Survivin (96-014), Tyrosinase (369-377, 371D), WT1 (10-18), WT1 (137-145), or WT1 (126-134).
110. The MBM of any one of embodiments 104 to 109, wherein the HLA-bound peptide is PRAME (425-433).
111. The MBM of any one of embodiments 104 to 109, wherein the HLA-bound peptide is MAGE-A4 (230-239).
112. The MBM of any one of embodiments 104 to 109, wherein the HLA-bound peptide is MAGE-A4 (286-294).
113. The MBM of any one of embodiments 103 to 112, wherein the HLA-bound peptide antigen targeting moiety (a) comprises the (i) CDR or (ii) VH and VL sequences of an antibody or TCR set forth in Table P-2 or (b) competes with an antibody set forth in Table P-2 for binding to its target.
114. The MBM of any one of embodiments 1 to 82, wherein the second antigen binding domain comprises means for binding a tumor antigen.
115. The MBM of any one of embodiments 1 to 82, wherein the second antigen binding domain comprises means for binding human HLA A*02:01/GVYDGREHTV.
116. The MBM of any one of embodiments 1 to 82, wherein the second antigen binding domain comprises means for binding human HLA A*02:01/KVLEHVVRV.
117. The MBM of any one of embodiments 1 to 116, wherein the second antigen binding domain is an scFv.
118. The MBM of embodiment 117, wherein the scFv (of the second antigen binding domain) has the configuration, in N- to C-terminal orientation, VL-linker-VH.
119. The MBM of embodiment 117, wherein the scFv (of the second antigen binding domain) has the configuration, in N- to C-terminal orientation, VH-linker-VL.
120. The MBM of any one of embodiments 117 to 119, wherein the VH of the scFv (of the second antigen binding domain) comprises identical CDRs to the VH of the second Fab.
121. The MBM of any one of embodiments 117 to 119, wherein the VL of the scFv (of the second antigen binding domain) comprises identical CDRs to the VL of the second Fab.
122. The MBM of any one of embodiments 1 to 116, wherein the second antigen binding domain is an sdVH.
123. The MBM of any one of embodiments 1 to 116, wherein the second antigen binding domain is a VHH.
124. The MBM of any one of embodiments 1 to 123, wherein the second Fab is an antigen targeting moiety.
125. The MBM of embodiment 124, wherein the second Fab is a tumor antigen targeting moiety.
126. The MBM of embodiment 125, wherein the second Fab specifically binds to a tumor antigen set forth in Table A.
127. The MBM of embodiment 125, wherein the second Fab specifically binds to a tumor antigen set forth in Table P-1.
128. The MBM of embodiment 125, wherein the second Fab specifically binds to a tumor antigen set forth in Table P-2.
129. The MBM of embodiment 125, wherein the second Fab (a) comprises the (i) CDR or (ii) VH and VL sequences of an antibody or TCR set forth in Table P-2 or (b) competes with an antibody or TCR set forth in Table P-2 for binding to its target.
130. The MBM of any one of embodiments 124 to 129, wherein the antigen targeting moiety is a low-density antigen targeting moiety.
131. The MBM of embodiment 130, wherein the low-density antigen is an antigen expressed at no more than 5000 copies per cell.
132. The MBM of embodiment 130, wherein the low-density antigen is an antigen expressed at no more than 4000 copies per cell.
133. The MBM of embodiment 130, wherein the low-density antigen is an antigen expressed at no more than 3000 copies per cell.
134. The MBM of embodiment 130, wherein the low-density antigen is an antigen expressed at no more than 2000 copies per cell.
135. The MBM of embodiment 130, wherein the low-density antigen is an antigen expressed at no more than 1000 copies per cell.
136. The MBM of embodiment 130, wherein the low-density antigen is an antigen expressed at no more than 500 copies per cell.
137. The MBM of embodiment 130, wherein the low-density antigen is an antigen expressed at no more than 250 copies per cell.
138. The MBM of embodiment 130, wherein the low-density antigen is an antigen expressed at no more than 100 copies per cell.
139. The MBM of embodiment 130, wherein the low-density antigen is an antigen expressed at no more than 50 copies per cell.
140. The MBM of embodiment 130, wherein the low-density antigen is an antigen expressed at no more than 40 copies per cell.
141. The MBM of embodiment 130, wherein the low-density antigen is an antigen expressed at no more than 30 copies per cell.
142. The MBM of embodiment 130, wherein the low-density antigen is an antigen expressed at no more than 20 copies per cell.
143. The MBM of embodiment 130, wherein the low-density antigen is an antigen expressed at no more than 10 copies per cell.
144. The MBM of any one of embodiments 124 to 143, wherein the antigen targeting moiety is an HLA-bound peptide antigen targeting moiety.
145. The MBM of embodiment 144, wherein the HLA-bound peptide is a viral peptide.
146. The MBM of embodiment 145, wherein the viral peptide is HPV16 E7 (11-20), HPV E716 (11-19), HPV16 E7 (82-90), CMV pp65 (495-503), HIV P17 (77-85), HIV RT (896-904), HIV GAG (41-49), or ENV (183-191).
147. The MBM of embodiment 145, wherein the viral peptide is a peptide set forth in Table P-1.
148. The MBM of any one of embodiments 144 to 147, wherein the HLA-bound peptide is a tumor-associated peptide.
149. The MBM of embodiment 148, wherein the tumor-associated peptide is LCMV derived peptide gp33-41, ACP3 (PAP) (112-120), AFP (58-68), AFP (173-181), APF (126-134), BALF(276-284), CEA (571-579), CMV pp65 (495-503), DNTT (475-483), DNTT (250-258), EBNA-1 (562-570), FLU-M1 (58-66), gp100 (154-162), gp100 (209-217), HBV Core (18-27), Her2/neu (369-377; V2v9); HPV16 E7 (11-20), HPV E716 (11-19), HPV16 E7 (82-90), IGF2BP3 (552-560), KLK4 (11-19), KLK4 (215-223), KLK4 (105-113), KRAS (6-14; G12V), KRAS (4-12; K5L/G12V), KRAS (5-14; G12V), KRAS (5-14; G12D), KRAS (10-18; G12D), KRAS (10-19; G12D), KRAS (8-16; G12D), KRAS (8-16; G12D), KRAS (7-16; G12D), KRAS (7-16; G12D), LMP1 (125-133), LMP2 (426-434), MAGE-A3 (112-120), MAGE-A3 (168-176), MAGE-A3 (271-279), MAGE-A3 (108-116), MAGE-A4 (230-239), MAGE-A4 (286-294), MAGE-A10 (137-145), MAGE-A10 (254-262), MAGE-A10 (310-318), MPO (132-140), MPO (549-557), MPO (571-579), NYESO1 (157-165, C165A), NYESO1 (157-165, C165V), p54 WT (264-272), PAP-3 (136-143), PRAME (425-433), PRAME (312-320), PSA (159-169), PSA (241-249), PSA (79-89), PSA (170-178), PSA (82-90), PSMA (4-12), PSMA (135-145), Survivin (96-014), Tyrosinase (369-377, 371D), WT1 (10-18), WT1 (137-145), or WT1 (126-134).
150. The MBM of any one of embodiments 144 to 149, wherein the HLA-bound peptide is PRAME (425-433).
151. The MBM of any one of embodiments 144 to 149, wherein the HLA-bound peptide is MAGE-A4 (230-239).
152. The MBM of any one of embodiments 144 to 149, wherein the HLA-bound peptide is MAGE-A4 (286-294).
153. The MBM of any one of embodiments 144 to 149, wherein the HLA-bound peptide antigen targeting moiety (a) comprises the (i) CDR or (ii) VH and VL sequences of an antibody or TCR set forth in Table P-2 or (b) competes with an antibody set forth in Table P-2 for binding to its target.
154. The MBM of any one of embodiments 1 to 123, wherein the second Fab comprises means for binding a tumor antigen.
155. The MBM of any one of embodiments 1 to 123, wherein the second Fab comprises means for binding human HLA A*02:01/GVYDGREHTV.
156. The MBM of any one of embodiments 1 to 123, wherein the second Fab comprises means for binding human HLA A*02:01/KVLEHVVRV.
157. The MBM of any one of embodiments 1 to 156, wherein the second antigen binding domain and the second Fab specifically bind to the same target.
158. The MBM of any one of embodiments 1 to 156, wherein the second antigen binding domain and the second Fab specifically bind to different targets.
159. The MBM of any one of embodiments 1 to 156, wherein the MBM has reduced binding to anti-drug antibodies as measured by an ADA reactivity assay as presented in Section 8.1.3 relative to a control antigen-binding molecule.
160. An MBM, which is optionally an MBM of any one of embodiments 1 to 159, the MBM comprising:
   (a) a first polypeptide chain comprising, in an N- to C-terminal orientation (i) a first antigen binding domain (e.g., an scFv or sdAb) that specifically binds to a first target (e.g., a T cell antigen such as CD3); (ii) an optional first linker; (iii) a first dimerization moiety; (iv) an optional second linker; and (v) a first portion of a first Fab that specifically binds to the first target;
   (b) a second polypeptide chain comprising, in an N- to C-terminal orientation (i) a second antigen binding domain (e.g., an scFv or an sdAb) that specifically binds to a second target (e.g., a tumor antigen, low-density antigen, or HLA-bound peptide antigen); (ii) an optional third linker; (iii) a second dimerization moiety; (iv) an optional fourth linker; and (v) a first portion of a second Fab that specifically binds to the second target;
   (c) a third polypeptide chain comprising a second portion of the first Fab associated with the first portion of the first Fab; and
   (d) a fourth polypeptide chain comprising a second portion of the second Fab associated with the first portion of the second Fab.
161. An MBM, which is optionally an MBM of any one of embodiments 1 to 159, the MBM comprising:
   (a) a first polypeptide chain comprising, in an N- to C-terminal orientation (i) an scFv that specifically binds to CD3; (ii) an optional first linker; (iii) an Fc domain; (iv) an optional second linker; (v) a first VH domain; and (vi) and a first CH1 domain;
   (b) a second polypeptide chain comprising, in an N- to C-terminal orientation (i) a an scFv that specifically binds to a tumor antigen, low-density antigen, or HLA-bound peptide antigen; (ii) an optional third linker; (iii) a second Fc domain; (iv) an optional fourth linker; (v) a second VH domain; and (vi) a second CH1 domain;
   (c) a third polypeptide chain comprising a first VL domain and a first CL domain associated with the first VH domain and the first CH1 domain to form a first Fab that specifically binds to CD3; and
   (d) a fourth polypeptide chain comprising a second VL domain and a second CL domain associated with the second VH domain and the second CH1 domain to form a second Fab that specifically binds to the tumor antigen, low-density antigen, or HLA-bound peptide antigen.
162. An MBM, which is optionally an MBM of any one of embodiments 1 to 161, which has the configuration depicted in FIG. 1A.
163. An MBM, which is optionally an MBM of any one of embodiments 1 to 161, which has the configuration depicted in FIG. 1B.
164. An MBM, which is optionally an MBM of any one of embodiments 1 to 161, which has the configuration depicted in FIG. 1C.
165. A nucleic acid or plurality of nucleic acids encoding the MBM of any one of embodiments 1 to 164.
166. A host cell engineered to express the MBM of any one of embodiments 1 to 164.
167. A cell transfected with one or more expression vectors comprising one or more nucleic acid sequences encoding the MBM molecule of any one of embodiments 1 to 164 under the control of one or more promoters.
168. A method of producing the MBM of any one of embodiments 1 to 164, comprising culturing the host cell of embodiment 166 or 167 and recovering the MBM expressed thereby.
169. A method of killing a cancer cell comprising administering to the cell the MBM of any one of embodiments 1 to 164.
170. The method of embodiment 169, wherein the cell expresses less than 5000, 4000, 3000, 2000, 1000, 500, 250, 100, 50, 40, 30, 20, or 10 copies of the antigen targeted by the antigen targeting moiety.
171. A method of activating a T cell comprising administering to the T cell the MBM of any one of embodiments 1 to 164.
172. A pharmaceutical composition comprising the MBM of any one of embodiments 1 to 164 and an excipient.
173. The pharmaceutical composition of embodiment 172, further comprising a multispecific antigen binding molecule comprising a tumor antigen targeting moiety and a TCA targeting moiety.
174. The pharmaceutical composition of embodiment 173, wherein the tumor antigen targeting moiety of the multispecific antigen binding molecule is an EGFR targeting moiety.
175. The pharmaceutical composition of embodiment 174, wherein the EGFR targeting moiety specifically binds to human EGFR.
176. The pharmaceutical composition of any one of embodiments 173 to 175, wherein the TCA targeting moiety of the multispecific antigen binding molecule is a CD28 targeting moiety.
177. The pharmaceutical composition of embodiment 176, wherein the CD28 targeting moiety specifically binds to human CD28.
178. The pharmaceutical composition of any one of embodiments 172 to 177, further comprising an immune checkpoint inhibitor.
179. The pharmaceutical composition of embodiment 178, wherein the immune checkpoint inhibitor is an anti-PD1 antibody.
180. A method of treating cancer, comprising administering to a subject in need thereof a multispecific binding molecule (MBM) comprising:
   (a) a first polypeptide chain comprising, in an N- to C-terminal orientation (i) a first antigen binding domain that specifically binds to a T cell antigen; (ii) an optional first linker; (iii) a first dimerization moiety; (iv) an optional second linker; and (v) a first portion of a first Fab that specifically binds to a T cell antigen;
   (b) a second polypeptide chain comprising, in an N- to C-terminal orientation (i) a second antigen binding domain that specifically binds to a tumor antigen; (ii) an optional third linker; (iii) a second dimerization moiety (iv) an optional fourth linker; and (v) a first portion of a second Fab that specifically binds to a tumor antigen;
   (c) a third polypeptide chain comprising a second portion of the first Fab associated with the first portion of the first Fab; and
   (d) a fourth polypeptide chain comprising a second portion of the second Fab associated with the first portion of the second Fab.
181. A method of inhibiting growth of a tumor cell in a subject, comprising administering to a subject a multispecific binding molecule (MBM) comprising:
   (a) a first polypeptide chain comprising, in an N- to C-terminal orientation (i) a first antigen binding domain that specifically binds to a T cell antigen; (ii) an optional first linker; (iii) a first dimerization moiety; (iv) an optional second linker; and (v) a first portion of a first Fab that specifically binds to a T cell antigen;
   (b) a second polypeptide chain comprising, in an N- to C-terminal orientation (i) a second antigen binding domain that specifically binds to a tumor antigen; (ii) an optional third linker; (iii) a second dimerization moiety (iv) an optional fourth linker; and (v) a first portion of a second Fab that specifically binds to a tumor antigen;
   (c) a third polypeptide chain comprising a second portion of the first Fab associated with the first portion of the first Fab; and
   (d) a fourth polypeptide chain comprising a second portion of the second Fab associated with the first portion of the second Fab.
182. A method of stimulating proliferation of cancer antigen specific T cells, comprising administering to a subject in need thereof a multispecific binding molecule (MBM) comprising:
   (a) a first polypeptide chain comprising, in an N- to C-terminal orientation (i) a first antigen binding domain that specifically binds to a T cell antigen; (ii) an optional first linker; (iii) a first dimerization moiety; (iv) an optional second linker; and (v) a first portion of a first Fab that specifically binds to a T cell antigen;
   (b) a second polypeptide chain comprising, in an N- to C-terminal orientation (i) a second antigen binding domain that specifically binds to a tumor antigen; (ii) an optional third linker; (iii) a second dimerization moiety (iv) an optional fourth linker; and (v) a first portion of a second Fab that specifically binds to a tumor antigen;
   (c) a third polypeptide chain comprising a second portion of the first Fab associated with the first portion of the first Fab; and
   (d) a fourth polypeptide chain comprising a second portion of the second Fab associated with the first portion of the second Fab.
183. The method of any one of embodiments 180 to 182, wherein the MBM is the MBM of any one of embodiments 1 to 164.
184. The method of any one of embodiments 180 to 183, wherein the MBM is administered by administration of the pharmaceutical composition of any one of embodiments 172 to 179.
185. The method of any one of embodiments 180 to 183, further comprising administering to the subject a multispecific antigen binding molecule comprising a tumor antigen targeting moiety and a TCA targeting moiety.
186. The method of embodiment 185, wherein the tumor antigen targeting moiety of the multispecific antigen binding molecule is an EGFR targeting moiety.
187. The method of embodiment 187, wherein the EGFR targeting moiety specifically binds to human EGFR.
188. The method of any one of embodiments 185 to 187, wherein the TCA targeting moiety of the multispecific antigen binding molecule is a CD28 targeting moiety.
189. The method of embodiment 188, wherein the CD28 targeting moiety specifically binds to human CD28.
190. The method of any one of embodiments 185 to 189, wherein the MBM and the multispecific antigen binding molecule are administered in the same composition.
191. The method of any one of embodiments 185 to 189, wherein the MBM and the multispecific antigen binding molecule are administered in different compositions.
192. The method of any one of embodiments 180 to 191, further comprising administering to the subject an immune checkpoint inhibitor.
193. The method of embodiment 192, wherein the immune checkpoint inhibitor is an anti-PD1 antibody.

### 8. EXAMPLES

### 8.1. Materials and Methods

### 8.1.1. Design and Production of Binding Molecules

Various binding molecule constructs were designed. An overview of the constructs generated and evaluated in the below Examples is provided in Table E-1. Each construct is described by its two half antibodies.

TABLE E-1
Binding Molecules

| Structure | Description |
|---|---|
| (CD3)scFv-Fc-(CD3)Fab × | Left half antibody |
| (MAGEA4(230-239))scFv-Fc- | (N- to C-terminal orientation) |
| (MAGEA4(230-239))Fab | (a) scFv that binds to CD3; |
| | (b) Fc domain; |
| | (c) Fab that binds to CD3 |
| | Right half antibody |
| | (N- to C-terminal orientation) |
| | (a) scFv that binds to HLA A*02:01/GVYDGREHTV; |
| | (b) Fc domain; |
| | (c) Fab that binds to HLA A*02:01/GVYDGREHTV |
| (CD3)scFv-Fc-(CD3)Fab × | Left half antibody |
| (MAGEA4(230-239))scFv-Fc- | (N- to C-terminal orientation) |
| (MAGEA4(286-294))Fab | (a) scFv that binds to CD3; |
| | (b) Fc domain; |
| | (c) Fab that binds to CD3 |
| | Right half antibody |
| | (N- to C-terminal orientation) |
| | (a) scFv that binds to HLA A*02:01/GVYDGREHTV; |
| | (b) Fc domain; |
| | (c) Fab that binds to HLA A*02:01/KVLEHVVRV |
| (CD3)Fab-Fc-(CD3)scFv × | Left half antibody |
| (MAGEA4(286-294))Fab-Fc- | (N- to C-terminal orientation) |
| (MAGEA4(230-239))scFv | (a) Fab that binds to CD3; |
| | (b) Fc domain; |
| | (c) scFv that binds to CD3 |
| | Right half antibody |
| | (N- to C-terminal orientation) |
| | (a) Fab that binds to HLA A*02:01/KVLEHVVRV; |
| | (b) Fc domain; |
| | (c) scFv that binds to HLA A*02:01/GVYDGREHTV |
| (CD3)Fab-Fc-(CD3)scFv × | Left half antibody |
| (MAGEA4(230-239))Fab-Fc- | (N- to C-terminal orientation) |
| (MAGEA4(230-239))scFv | (a) Fab that binds to CD3; |
| | (b) Fc domain; |
| | (c) scFv that binds to CD3 |
| | Right half antibody |
| | (N- to C-terminal orientation) |
| | (a) Fab that binds to HLA A*02:01/GVYDGREHTV; |
| | (b) Fc domain; |
| | (c) scFv that binds to HLA A*02:01/GVYDGREHTV |
| (CD3)Fab-Fc-(CD3)Fab × | Left half antibody |
| (MAGEA4(230-239))Fab-Fc- | (N- to C-terminal orientation) |
| (MAGEA4(230-239))Fab | (a) Fab that binds to CD3; |
| | (b) Fc domain; |
| | (c) Fab that binds to CD3 |
| | Right half antibody |
| | (N- to C-terminal orientation) |
| | (a) Fab that binds to HLA A*02:01/GVYDGREHTV; |
| | (b) Fc domain; |
| | (c) Fab that binds to HLA A*02:01/GVYDGREHTV |
| (CD3)Fab-Fc × | Left half antibody |
| MAGEA4(230-239)Fab-Fc | (N- to C-terminal orientation) |
| | (a) Fab that binds to CD3; |
| | (b) Fc domain |
| | Right half antibody |
| | (N- to C-terminal orientation) |
| | (a) Fab that binds to HLA A*02:01/GVYDGREHTV; |
| | (b) Fc domain |
| (CD3)scFv-Fc-(CD3)Fab × | Left half antibody |
| (Pan-HLA)scFv-Fc-(Pan- | (N- to C-terminal orientation) |
| HLA)Fab | (a) scFv that binds to CD3; |
| | (b) Fc domain; |
| | (c) Fab that binds to CD3 |
| | Right half antibody |
| | (N- to C-terminal orientation) |
| | (a) scFv that binds to HLA A*02:01; |
| | (b) Fc domain; |
| | (c) Fab that binds to HLA A*02:01 |
| (CD3)scFv-Fc × | Left half antibody |
| (Pan-HLA)Fab-Fc | (N- to C-terminal orientation) |
| | (a) scFv that binds to CD3; |
| | (b) Fc domain |
| | Right half antibody |
| | (N- to C-terminal orientation) |
| | (a) Fab that binds to HLA A*02:01; |
| | (b) Fc domain |
| (CD3)Fab-Fc-(CD3)scFv × | Left half antibody |
| (Non-targeting)Fab-Fc-(Non- | (N- to C-terminal orientation) |
| targeting)scFv | (a) Fab that binds to CD3; |
| | (b) Fc domain; |
| | (c) scFv that binds to CD3 |
| | Right half antibody |
| | (N- to C-terminal orientation) |
| | (a) non-targeting Fab; |
| | (b) Fc domain; |
| | (c) non-targeting scFv |

All constructs were expressed in Expi293F™ cells by transient transfection (Thermo Fisher Scientific). Proteins in Expi293F supernatant were purified using the ProteinMaker system (Protein BioSolutions, Gaithersburg, MD) with HiTrap MabSelect SuRe Protein A columns (GE Healthcare). After single step elution with IgG Elution Buffer (Thermo Fisher Scientific), the proteins were neutralized, dialyzed into a final buffer of phosphate buffered saline (PBS) with 5% glycerol, aliquoted and stored at −80° C.

### 8.1.2. Cytotoxicity Assay

Target cell lines were resuspended in PBS and stained with CellTrace™ Violet (Thermo Fisher Scientific) based on manufacturer protocols. Target cells were resuspended in RPMI media containing 10% FBS and penicillin-streptomycin-glutamine supplementation (R10 media) and plated in a 96-well plate to provide 10,000 cells per well. Effector cells (isolated T cells) were plated to provide 60,000 cells per well, so that the Effector:Target cell ratio was 6:1. MBMs were diluted with a ratio of 1:10 and added to the assay plates with a final starting concentration of 6.7×10⁻⁰⁸ M in R10 media.

The plates were incubated in a cell culture incubator for 72 hours or longer. The supernatant was then removed and spun down at 300×g for 4 minutes to pellet all cells in suspension. The tumor cells were washed and harvested with trypsin, and combined with the suspension cell pellet. The final cell pellet was washed with PBS and stained with LIVE/DEAD Fixable Near-IR Dead Cell Stain Kit (Thermo Fisher Scientific). After 2 washes with PBS, cells were resuspended in FACS wash and analyzed on a cytometer such as the BD FACSCelesta™ instrument or BD FACSCanto™ II instrument. For the assessment of killing specificity, cells were gated on live Violet labeled populations. The percentage of the live population was recorded and used for the calculation of survival. Results were analyzed by FlowJo™. All appropriate compensation samples and fluorescence minus one (FMO) controls were included.

### 8.1.3. ADA Reactivity Assay

In order to assess antigenicity, molecules were tested in an ADA reactivity assay using a SMCxPro™ Immunogenicity method for measuring binding to pre-existing anti-drug antibodies. Briefly, antibodies were labeled with biotin and Alexa647 according to the Millipore SMC™ Immunogenicity Plate Based Assay Development Kit protocol (Catalog #03-0189-00). Biotinylated-antibody at 1 μg/mL, Alexa647-antibody at 0.063 ug/mL, and human or monkey serum diluted 1:10 were combined 1:1:1 and incubated overnight shaking at 4° C. After incubation, the bridged complex was transferred to a pre-blocked streptavidin-coated assay plate and incubated for 1 hour shaking at RT. The plate was washed 6 times in 1× Wash Buffer. Elution Buffer B was added to the assay plate and incubated for 10 minutes shaking at RT. After 10 minutes the reaction was quenched with Buffer C. The neutralized solution was transferred to an SMCxPro™ Aurora plate, sealed, and read on the SMCxPro™

### 8.2. Example 1: Cytotoxic Potency of Binding Molecule Formats Against A375 Cells

Wild-type A375 cells express ˜500 copies of HLA-A2 MAGE-A4 (230-239) per cell. These cells were used for cytotoxicity analysis.

Binding molecules were generated as described in Section 8.1.1; those evaluated in this example are shown in Table E-2, below. CD3-binding sequences (VH and VL) were identical across all constructs. MAGEA4(230-239) binding sequences (VH and VL) were identical across all constructs.

TABLE E-2
Binding Molecules

| Construct | Structure | Description |
|---|---|---|
| 1 | (CD3)Fab-Fc × | Left half antibody |
| | MAGEA4(230-239)Fab-Fc | (N- to C-terminal orientation) |
| | | (a) Fab that binds to CD3; |
| | | (b) Fc domain |
| | | Right half antibody |
| | | (N- to C-terminal orientation) |
| | | (a) Fab that binds to HLA A*02:01/GVYDGREHTV; |
| | | (b) Fc domain |
| 2 | (CD3)Fab-Fc-(CD3)scFv × | Left half antibody |
| | (MAGEA4(230-239))Fab- | (N- to C-terminal orientation) |
| | Fc-(MAGEA4(230- | (a) Fab that binds to CD3; |
| | 239))scFv | (b) Fc domain; |
| | | (c) scFv that binds to CD3 |
| | | Right half antibody |
| | | (N- to C-terminal orientation) |
| | | (a) Fab that binds to HLA A*02:01/GVYDGREHTV; |
| | | (b) Fc domain; |
| | | (c) scFv that binds to HLA A*02:01/GVYDGREHTV |
| 3 | (CD3)scFv-Fc-(CD3)Fab × | Left half antibody |
| | (MAGEA4(230-239))scFv- | (N- to C-terminal orientation) |
| | Fc-(MAGEA4(230-239))Fab | (a) scFv that binds to CD3; |
| | | (b) Fc domain; |
| | | (c) Fab that binds to CD3 |
| | | Right half antibody |
| | | (N- to C-terminal orientation) |
| | | (a) scFv that binds to HLA A*02:01/GVYDGREHTV; |
| | | (b) Fc domain; |
| | | (c) Fab that binds to HLA A*02:01/GVYDGREHTV |

A cytotoxicity assay was performed by incubating wild-type A375 cells with the shown molecules for 72 hours as described in Section 8.1.2.

Construct 3 displayed significantly enhanced cytotoxicity against wild-type A375 cells relative to both construct 1 and construct 2 (FIG. 2), highlighting the impact of targeting moiety format on potency and efficacy.

### 8.3. Example 2: Cytotoxic Potency of MBMs Against Cells with Different Copy Numbers of HLA-A2 MAGE-A4

Wild-type A375 cells express ˜500 copies of HLA-A2 MAGE-A4 (230-239) per cell and ˜500 copies of HLA-A2 MAGE-A4 (286-294) per cell. “Clone 8” A375 cells were generated from a clone expressing ˜20 copies of HLA-A2 MAGE-A4 (230-239) per cell and ˜15 copies of HLA-A2 MAGE-A4 (286-294) per cell. These cells were used for cytotoxicity analysis.

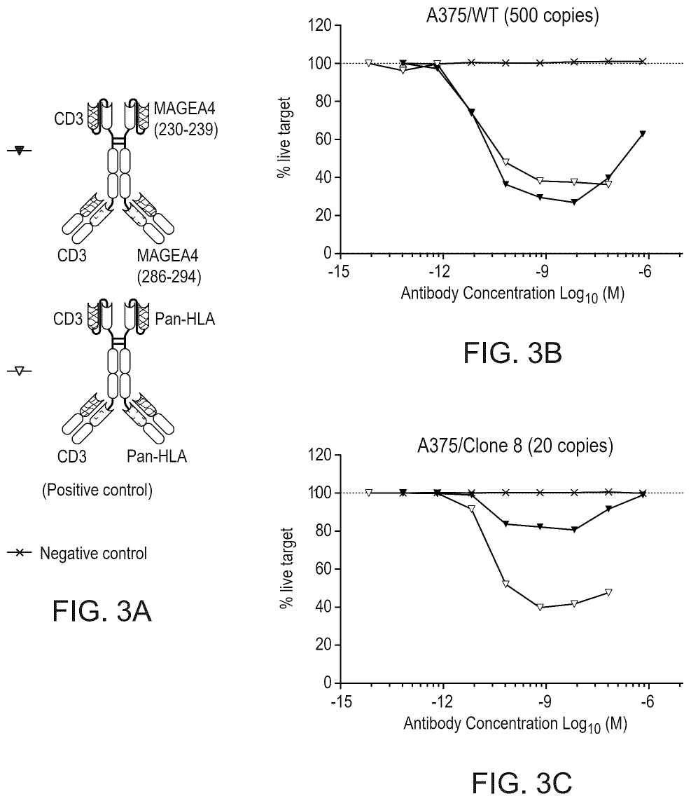

Binding molecules were generated as described in Section 8.1.1; those evaluated in this example are shown in Table E-3, below, and depicted in FIG. 3A. CD3-binding sequences (VH and VL) were identical across all constructs.

TABLE E-3
Binding Molecules

| Construct | Structure | Description |
|---|---|---|
| 1 | (CD3)scFv-Fc-(CD3)Fab × | Left half antibody |
| | (MAGEA4(230-239))scFv- | (N- to C-terminal orientation) |
| | Fc-(MAGEA4(286- | (a) scFv that binds to CD3; |
| | 294))Fab | (b) Fc domain; |
| | | (c) Fab that binds to CD3 |
| | | Right half antibody |
| | | (N- to C-terminal orientation) |
| | | (a) scFv that binds to HLA |
| | | A*02:01/GVYDGREHTV; |
| | | (b) Fc domain; |
| | | (c) Fab that binds to HLA A*02:01/KVLEHVVRV |
| 2 | (CD3)scFv-Fc-(CD3)Fab × | Left half antibody |
| (Positive | (Pan-HLA)scFv-Fc-(Pan- | (N- to C-terminal orientation) |
| Control) | HLA)Fab | (a) scFv that binds to CD3; |
| | | (b) Fc domain; |
| | | (c) Fab that binds to CD3 |
| | | Right half antibody |
| | | (N- to C-terminal orientation) |
| | | (a) scFv that binds to HLA A*02:01; |
| | | (b) Fc domain; |
| | | (c) Fab that binds to HLA A*02:01 |

A cytotoxicity assay was performed by incubating wild-type and “Clone 8” A375 cells with the shown molecules for 96 hours as described in Section 8.1.2. Construct 1 (targeting MAGEA4(230-239)) showed cytotoxicity against both wild-type A375 cells (FIG. 3B) and Clone 8 A375 cells (FIG. 3C), demonstrating efficacy in inducing tumor cell killing with target expression as low as 20 copies per cell.

### 8.4. Example 3: Evaluation of Pre-Existing ADA Reactivity

Binding molecules were generated as described in Section 8.1.1; those evaluated in this example are shown in Table E-4, below. ADA reactivity of certain MBMs was assessed as described in Section 8.1.3. CD3-binding sequences (VH and VL) were identical across all constructs. MAGEA4(230-239) binding sequences (VH and VL) were identical across all constructs.

TABLE E-4
Binding Molecules

| Construct | Structure | Description |
|---|---|---|
| 1 | (CD3)Fab-Fc-(CD3)scFv × | Left half antibody |
| | (MAGEA4(286-294))Fab-Fc- | (N- to C-terminal orientation) |
| | (MAGEA4(230-239))scFv | (a) Fab that binds to CD3; |
| | | (b) Fc domain; |
| | | (c) scFv that binds to CD3 |
| | | Right half antibody |
| | | (N- to C-terminal orientation) |
| | | (a) Fab that binds to HLA A*02:01/KVLEHVVRV; |
| | | (b) Fc domain; |
| | | (c) scFv that binds to HLA A*02:01/GVYDGREHTV |
| 2 | (CD3)Fab-Fc × | Left half antibody |
| | MAGEA4(230-239)Fab-Fc | (N- to C-terminal orientation) |
| | | (a) Fab that binds to CD3; |
| | | (b) Fc domain |
| | | Right half antibody |
| | | (N- to C-terminal orientation) |
| | | (a) Fab that binds to HLA A*02:01/GVYDGREHTV; |
| | | (b) Fc domain |
| 3 | (CD3)scFv-Fc-(CD3)Fab × | Left half antibody |
| | (MAGEA4(230-239))scFv-Fc- | (N- to C-terminal orientation) |
| | (MAGEA4(286-294))Fab | (a) scFv that binds to CD3; |
| | | (b) Fc domain; |
| | | (c) Fab that binds to CD3 |
| | | Right half antibody |
| | | (N- to C-terminal orientation) |
| | | (a) scFv that binds to HLA A*02:01/GVYDGREHTV; |
| | | (b) Fc domain; |
| | | (c) Fab that binds to HLA A*02:01/KVLEHVVRV |

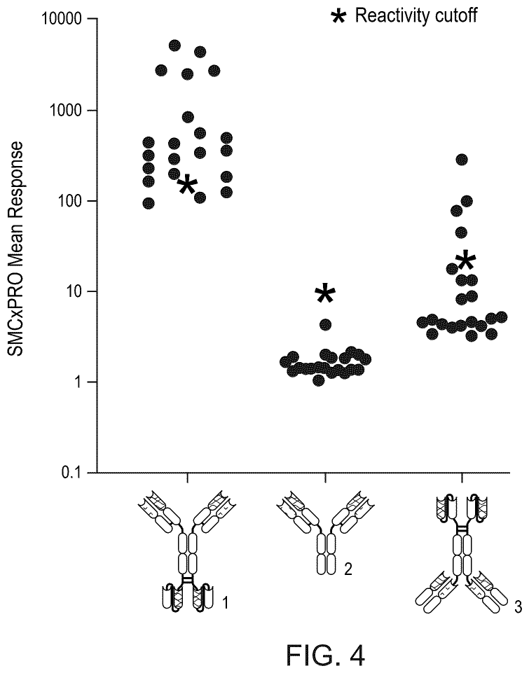

Construct 3 displayed significantly lower levels of reactivity to pre-existing ADA relative to construct 1 (FIG. 4), with the vast majority of samples tested below the reactivity cutoff.

### 8.5. Example 4: Cytotoxic Potency of Binding Molecules Against A375 Cells

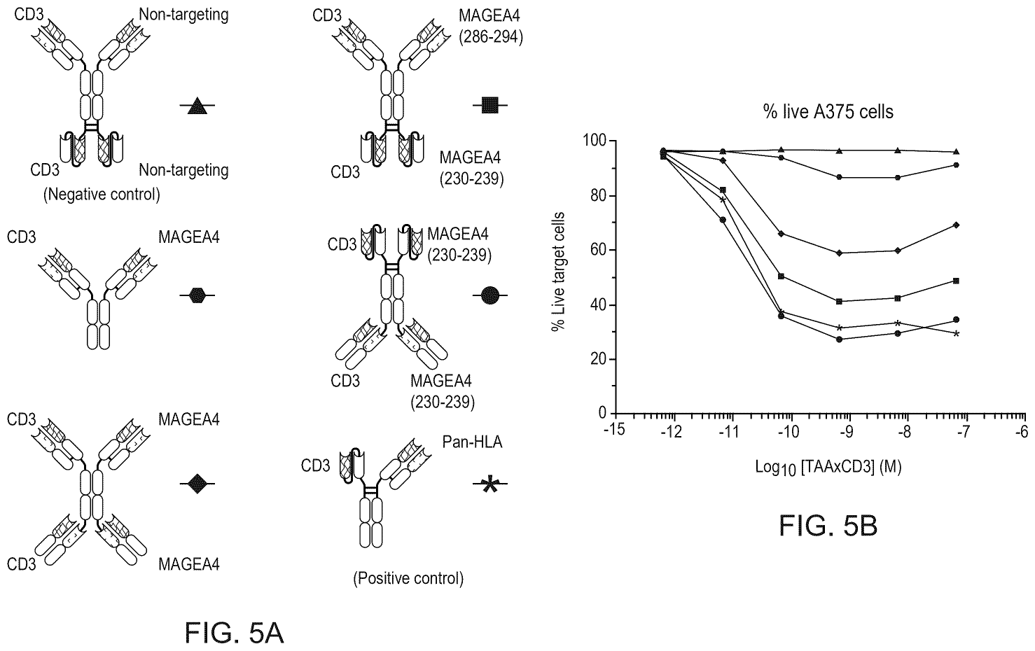

Binding molecules were generated as described in Section 8.1.1; those evaluated in this example are shown in Table E-5, below, and depicted in FIG. 5A. CD3-binding sequences (VH and VL) were identical across all constructs. MAGEA4(230-239) binding sequences (VH and VL) were identical across all constructs.

TABLE E-5
Binding Molecules

| Construct | Structure | Description |
|---|---|---|
| 1 | (CD3)Fab-Fc-(CD3)scFv × | Left half antibody |
| (Negative | (Non-targeting)Fab-Fc-(Non- | (N- to C-terminal orientation) |
| Control) | targeting)scFv | (a) Fab that binds to CD3; |
| | | (b) Fc domain; |
| | | (c) scFv that binds to CD3 |
| | | Right half antibody |
| | | (N- to C-terminal orientation) |
| | | (a) non-targeting Fab; |
| | | (b) Fc domain; |
| | | (c) non-targeting scFv |
| 2 | (CD3)Fab-Fc × | Left half antibody |
| | MAGEA4(230-239)Fab-Fc | (N- to C-terminal orientation) |
| | | (a) Fab that binds to CD3; |
| | | (b) Fc domain |
| | | Right half antibody |
| | | (N- to C-terminal orientation) |
| | | (a) Fab that binds to HLA |
| | | A*02:01/GVYDGREHTV; |
| | | (b) Fc domain |
| 3 | (CD3)Fab-Fc-(CD3)Fab × | Left half antibody |
| | (MAGEA4(230-239))Fab-Fc- | (N- to C-terminal orientation) |
| | (MAGEA4(230-239))Fab | (a) Fab that binds to CD3; |
| | | (b) Fc domain; |
| | | (c) Fab that binds to CD3 |
| | | Right half antibody |
| | | (N- to C-terminal orientation) |
| | | (a) Fab that binds to HLA |
| | | A*02:01/GVYDGREHTV; |
| | | (b) Fc domain; |
| | | (c) Fab that binds to HLA |
| | | A*02:01/GVYDGREHTV |
| 4 | (CD3)Fab-Fc-(CD3)scFv × | Left half antibody |
| | (MAGEA4(286-294))Fab-Fc- | (N- to C-terminal orientation) |
| | (MAGEA4(230-239))scFv | (a) Fab that binds to CD3; |
| | | (b) Fc domain; |
| | | (c) scFv that binds to CD3 |
| | | Right half antibody |
| | | (N- to C-terminal orientation) |
| | | (a) Fab that binds to HLA |
| | | A*02:01/KVLEHVVRV; |
| | | (b) Fc domain; |
| | | (c) scFv that binds to HLA |
| | | A*02:01/GVYDGREHTV |
| 5 | (CD3)scFv-Fc-(CD3)Fab × | Left half antibody |
| | (MAGEA4(230-239))scFv-Fc- | (N- to C-terminal orientation) |
| | (MAGEA4(230-239))Fab | (a) scFv that binds to CD3; |
| | | (b) Fc domain; |
| | | (c) Fab that binds to CD3 |
| | | Right half antibody |
| | | (N- to C-terminal orientation) |
| | | (a) scFv that binds to HLA |
| | | A*02:01/GVYDGREHTV; |
| | | (b) Fc domain; |
| | | (c) Fab that binds to HLA |
| | | A*02:01/GVYDGREHTV |
| 6 | (CD3)scFv-Fc × | Left half antibody |
| (Positive | (Pan-HLA)Fab-Fc | (N- to C-terminal orientation) |
| Control) | | (a) scFv that binds to CD3; |
| | | (b) Fc domain |
| | | Right half antibody |
| | | (N- to C-terminal orientation) |
| | | (a) Fab that binds to HLA A*02:01; |
| | | (b) Fc domain |

A cytotoxicity assay was performed by incubating wild-type A375 cells with the shown molecules for 96 hours as described in Section 8.1.2. Consistent with the results described in Example 1, construct 5 showed the most potent cytotoxicity relative to constructs 2-4 (FIG. 5B), further emphasizing the impact of targeting moiety format on potency and efficacy.

1. A multispecific binding molecule (MBM) comprising:
(a) a first polypeptide chain comprising, in an N- to C-terminal orientation (i) a first antigen binding domain that specifically binds to a first target; (ii) first Fc domain; and (iii) a first portion of a first Fab that specifically binds to the first target;
(b) a second polypeptide chain comprising, in an N- to C-terminal orientation (i) a second antigen binding domain that specifically binds to a second target; (ii) a second Fc domain associated with the first Fc domain to form an Fc region; and (iii) a first portion of a second Fab that specifically binds to the second target;
(c) a third polypeptide chain comprising a second portion of the first Fab associated with the first portion of the first Fab; and
(d) a fourth polypeptide chain comprising a second portion of the second Fab associated with the first portion of the second Fab.

2. (canceled)

3. (canceled)

4. The MBM of any one of claim 1, wherein (a) the first portion of the first Fab comprises a VH domain and a CH1 domain and (b) the second portion of the first Fab comprises a VL domain and a CL domain.

5.-7. (canceled)

8. The MBM of claim 1, wherein (a) the first portion of the second Fab comprises a VH domain and a CH1 domain and (b) the second portion of the second Fab comprises a VL domain and a CL dom.

9.-11. (canceled)

12. The MBM of claim 1, wherein the first antigen binding domain and the first Fab are T cell antigen (TCA) targeting moieties.

13. The MBM of claim 12, wherein the first antigen binding domain and the first Fab are CD3 targeting moieties.

14. The MBM of claim 1, wherein the first antigen binding domain is an scFv.

15. The MBM of claim 14, wherein the scFv of the first antigen binding domain has the configuration, in N- to C-terminal orientation, VL-linker-VH.

16. (canceled)

17. (canceled)

18. The MBM of claim 1, wherein the second antigen binding domain and the second Fab are tumor antigen targeting moieties.

19. The MBM of claim 1, wherein the second antigen binding domain and the second Fab are low-density antigen targeting moieties.

20. The MBM of claim 19, wherein the low-density antigen is an antigen expressed at no more than 5000 copies per cell.

21. (canceled)

22. The MBM of claim 1, wherein the second antigen binding domain and the second Fab are HLA-bound peptide antigen targeting moieties.

23. The MBM of claim 22, wherein the HLA-bound peptide is a viral peptide.

24. The MBM of claim 23, wherein the viral peptide is HPV16 E7 (11-20), HPV E716 (11-19), HPV16 E7 (82-90), CMV pp65 (495-503), HIV P17 (77-85), HIV RT (896-904), HIV GAG (41-49), or ENV (183-191).

25.-27. (canceled)

28. The MBM of claim 1, wherein the second antigen binding domain is an scFv.

29. The MBM of claim 28, wherein the scFv of the second antigen binding domain has the configuration, in N- to C-terminal orientation, VL-linker-VH.

30. (canceled)

31. (canceled)

32. The MBM of claim 1, wherein the MBM has reduced binding to anti-drug antibodies as measured by an ADA reactivity assay relative to a control antigen-binding molecule.

33.-35. (canceled)

36. A nucleic acid or plurality of nucleic acids encoding the MBM of claim 1.

37. A host cell engineered to express the MBM of claim 1.

38. A method of producing an MBM, comprising culturing the host cell of claim 37 and recovering the MBM expressed thereby.

39. A method of killing a cancer cell comprising administering to the cell the MBM of claim 1.

40. A method of activating a T cell comprising administering to the T cell the MBM of claim 1.

41. A pharmaceutical composition comprising the MBM of claim 1 and an excipient.

42. The pharmaceutical composition of claim 41, further comprising a multispecific antigen binding molecule comprising a tumor antigen targeting moiety and a TCA targeting moiety.

43. (canceled)

44. (canceled)

45. A method of treating cancer, comprising administering to a subject in need thereof a multispecific binding molecule (MBM) comprising:
(a) a first polypeptide chain comprising, in an N- to C-terminal orientation (i) a first antigen binding domain that specifically binds to CD3; (ii) a first dimerization moiety; and (iii) a first portion of a first Fab that specifically binds to CD3;
(b) a second polypeptide chain comprising, in an N- to C-terminal orientation (i) a second antigen binding domain that specifically binds to a tumor antigen; (ii) a second dimerization moiety; and (iii) a first portion of a second Fab that specifically binds to a tumor antigen;
(c) a third polypeptide chain comprising a second portion of the first Fab associated with the first portion of the first Fab; and
(d) a fourth polypeptide chain comprising a second portion of the second Fab associated with the first portion of the second Fab.

46. A method of inhibiting growth of a tumor cell in a subject, comprising administering to a subject a multispecific binding molecule (MBM) comprising:
(a) a first polypeptide chain comprising, in an N- to C-terminal orientation (i) a first antigen binding domain that specifically binds to CD3; (ii) a first dimerization moiety; and (iii) a first portion of a first Fab that specifically binds to CD3;
(b) a second polypeptide chain comprising, in an N- to C-terminal orientation (i) a second antigen binding domain that specifically binds to a tumor antigen; (ii) a second dimerization moiety; and (iii) a first portion of a second Fab that specifically binds to a tumor antigen;
(c) a third polypeptide chain comprising a second portion of the first Fab associated with the first portion of the first Fab; and
(d) a fourth polypeptide chain comprising a second portion of the second Fab associated with the first portion of the second Fab.

47. A method of stimulating proliferation of cancer antigen specific T cells, comprising administering to a subject in need thereof a multispecific binding molecule (MBM) comprising:
(a) a first polypeptide chain comprising, in an N- to C-terminal orientation (i) a first antigen binding domain that specifically binds to CD3; (ii) a first dimerization moiety; and (iii) a first portion of a first Fab that specifically binds to CD3;
(b) a second polypeptide chain comprising, in an N- to C-terminal orientation (i) a second antigen binding domain that specifically binds to a tumor antigen; (ii) a second dimerization moiety; and (iii) a first portion of a second Fab that specifically binds to a tumor antigen;
(c) a third polypeptide chain comprising a second portion of the first Fab associated with the first portion of the first Fab; and
(d) a fourth polypeptide chain comprising a second portion of the second Fab associated with the first portion of the second Fab.

48.-50. (canceled)
